(19) United States
(12) Patent Application Publication
(10) Pub. No.: US 2020/0254041 A1
(43) Pub. Date: Aug. 13, 2020

(54) RAPID ONSET AND EXTENDED ACTION PLANT-BASED AND SYNTHETIC CANNABINOID FORMULATIONS

(71) Applicant: RECEPTOR HOLDINGS, INC., Seattle, WA (US)

(72) Inventors: Andrea Leone-Bay, Ridgefield, CT (US); Gregory Wesner, Bainbridge Island, WA (US)

(73) Assignee: Receptor Holdings, Inc., Seattle, WA (US)

(21) Appl. No.: 16/753,726

(22) PCT Filed: Oct. 5, 2018
(86) PCT No.: PCT/US18/54733

§ 371 Date: Apr. 3, 2020

Related U.S. Application Data
(60) Provisional application No. 62/568,705, filed on Oct. 5, 2017.

(51) Int. Cl. A61K 36/185; A61K 9/48; A61K 47/38; A61K 45/06; A61K 31/05; A61K 31/352

(52) U.S. Cl. CPC A61K 36/185; A61K 9/4891; A61K 9/4875; A61K 31/352; A61K 45/06; A61K 31/05; A61K 47/38

(57) ABSTRACT

Rapid onset and extended action plant-based medicinal compounds or nutritional supplements and synthetic cannabinoid formulations are described. Rapid onset is provided by N-acylated fatty amino acids and/or penetration enhancers. Extended action can be provided by one or more sustained-release systems.

### CROSS-REFERENCE TO RELATED APPLICATION

This application claims priority to 62/568,705 filed on Oct. 5, 2017, which is incorporated herein by reference in its entirety as if fully set forth herein.

### FIELD OF THE DISCLOSURE

The current disclosure provides rapid onset and extended action plant-based medicinal compounds or nutritional supplements and synthetic cannabinoid formulations. Rapid onset is provided by N-acylated fatty amino acids and/or penetration enhancers. Extended action can be provided by one or more sustained-release systems.

### BACKGROUND OF THE DISCLOSURE

Historically, the plant world has been the most important source of medicinal agents for the treatment of human and animal disease, and for use as preventative agents in maintaining good health. However, for at least the last 150 years, Western medicine has been dominated by synthetic chemical agents.

It is now being increasingly recognized, however, that many plants and plant extracts are highly effective agents for the prevention and treatment of disease. A single plant can possess a large number of pharmaceutically active agents, and extracts obtained therefrom can exert their activities on a variety of physiologic processes, increasing the range of the desired therapeutic effect.

As one example, U.S. Publication No. 2015/0050373 describes use of plants from the Calophyllum genus to treat metabolic disorders. Calophyllum is a flowering plant genus of around 180-200 species of tropical evergreen trees. The Calophyllum genus includes four subcategories: Calophyllum brasiliense, Calophyllum caledonicurn, Calophyllum inophyllum and Calophyllum soulattri. Calophyllum inophyllum, is a medium to large sized evergreen tree that averages 25-65 feet in height. Different medicinal uses of this plant have been reported in the literature, for example, decoction of the bark of this plant treats internal hemorrhages. The oil extracted from Calophyllum inophyllum seeds is used to treat rheumatoid arthritis or joint disorders; itching; eczema; pimples appearing on head; eye diseases; and kidney failure.

U.S. Publication No. 2014/0193345 describes use of the plants Uncaria tomentosa, Thymus vulgaris, Matricaria recutita, Salix alba, Calendula officinalis, Usnea barbata, Ligusticum porterii-osha, Gaultheria procumbens, Camellia sinensis, Vaccinium myrtillus, Melissa officinalis, Allium sativum, Camellia sinensis and Krameria triandra to treat mucosal lesions.

U.S. Publication No. 2010/0068297 describes use of the plants Punica granatum, Viburnum plicatum, Camellia sinensis, and Acer spp. as antimicrobials.

Numerous medical uses have also been identified for the cannabis plant. For example, delta-9-tetrahydrocannabinol (THC, also referred to as Dronabinol), an extract of the cannabis plant has been formulated in sesame oil for oral delivery. THC exhibits complex effects on the central nervous system (CNS), including central sympathomimetic activity. THC has been shown to have a marked appetite stimulant effect and has been used in the treatment of AIDS-related anorexia. THC demonstrates effects on appetite, mood, cognition, memory and perception. Furthermore, the drug has anti-emetic properties and is used to control nausea and vomiting associated with cancer chemotherapy. These effects appear to be dose related.

THC's efficacy in pain treatment has been described in Pharm. J. 259, 104, 1997 and in Pharm. Sci. 3, 546, 1997. Nabilone, a synthetic cannabinoid has also been reported to be an anti-emetic and anxiolytic, and also useful for treating pain of various etiologies such as multiple sclerosis (MS), peripheral neuropathy and spinal injuries (Lancet, 1995, 345, 579, Pharm. J. 259, 104, 1997; Baker & Pryce, Expert Opin Investig Drugs. 2003 April; 12(4):561-7)). THC has also been reported to be useful in the treatment of AIDS (J. Pain. Symptom Manage. 1995, 10, 89-97) when given orally.

Another cannabinoid with well-documented health benefits is cannabidiol (CBD). In contrast to THC, CBD does not exert psychoactive effects. CBD is reported to have antidepressant (Zanelati T, et al. Journal of Pharmacology. 2010. 159(1):122-8;), anti-anxiety (Resstel B M, et al. Br J Pharmacol. 2009. 156(1):181-188), anti-inflammatory (Vuolo F, et al. Mediators of Inflammation. 2015. 538670), and neuroprotective effects (Campos A C, et al. Pharmacol Res. 2016. 112:119-127).

Additional uses for the cannabis plant include treatment of addiction (De Vries, et al., Psychopharmacology (Berl). 2003 July; 168(1-2):164-9); ADHD (O'Connell and Che, Harm Reduction Journal. 2007; 4:16); alcoholism (Basavarajappa & Hungund, Alcohol. 2005 January-February; 40(1):15-24); Alzheimer's disease (Eubanks et al., Mol Pharm. 2006 November-December; 3(6):773-7); amyotrophic lateral sclerosis (ALS) (Raman et al., Amyotroph Lateral Scler Other Motor Neuron Disord. 2004 March; 5(1):33-9); anxiety (The British Journal of Psychiatry February 2001, 178 (2) 107-115); asthma (Tashkin et al., American Review of Respiratory Disease, 1975; 112, 377); auto-immune diseases (Lyman et al., J Neuroimmunol. 1989 June; 23(1):73-81); bacterial infections (Nissen et al., Fitoterapia. 2010 July; 81(5):413-9); bone loss (Bab et al., Ann Med. 2009; 41(8):560-7); brain injury/stroke (Shohami et al., Br J Pharmacol. 2011 August; 163(7):1402-10); cancer (Guindon & Hohmann, Br J Pharmacol. 2011 August; 163(7):1447-63); heart disease (Walsh et al., Br J Pharmacol. 2010 July; 160(5):1234-42); Huntington's disease (Lastres-Becker et al., J Neurochem. 2003 March; 84(5):1097-109); inflammation (AAPS J. 2009 March; 11(1): 109-119); Parkinson's disease (Sieradzan et al., Neurology. 2001 Dec. 11; 57(11):2108-11); and psoriasis (Trends Pharmacol Sci. 2009 August; 30(8): 411-420).

Additional documented uses for the cannabis plant include treating acquired hypothyroidism, acute gastritis, agoraphobia, ankyloses, arthritis, Asperger's syndrome, atherosclerosis, autism, bipolar disorder, blood disorders, cachexia, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Grave's disease, hepatitis, herpes, hypertension, impotence, incontinence, infant mortality, inflammatory bowel disease (IBD), insomnia, liver fibrosis, mad cow disease, menopause, migraine headaches, motion sickness, MRSA, muscular dystrophy, nail patella syndrome, neuroinflammation, nicotine addiction, obesity, obsessive compulsive disorder (OCD), pancreatitis, panic disorder, periodontal disease, phantom limb pain, poison ivy allergy, premenstrual syndrome (PMS), proximal myotonic myopathy, post-traumatic stress disorder (PTSD), Raynaud's disease, restless leg syndrome, schizophrenia, scleroderma, septic shock, shingles herpes zoster), sickle cell disease, seizures, sleep apnea, sleep disorders, stress, stuttering, temporomandibular joint disorder (TMJ), tension headaches, tinnitus, Tourette's syndrome, traumatic memories, wasting syndrome, and withdrawal.

Despite the numerous benefits associated with plant-based compounds and nutritional supplements, when administered in oral form, their onset of action is generally slow, which can detract from their usefulness in some instances. For example, after oral administration, THC has an onset of action of up to 1.5 hours and a peak effect at 2-4 hours. The duration of action is 4-6 hours. THC is almost completely absorbed (90-95%) after single oral doses. However, due to a combined effect of first pass hepatic metabolism and poor aqueous solubility (THC water solubility is 2.8 mg/L), only 4-20% of the administered dose reaches the systemic circulation. Therefore, oral consumption of cannabis is characterized by low bioavailability of cannabinoids, and slow onset of action. Thus, as this one example provides, there is room for improvement in the oral administration of plant-based compounds and nutritional supplements.

In addition to room for improvement in the time for onset to action, it can also be beneficial to provide extended action of plant-based compounds and nutritional supplements to extend the time of beneficial effects and reduce the need for repeat dosing.

### SUMMARY OF THE DISCLOSURE

The current disclosure provides rapid onset and extended action plant-based medicinal compounds and nutritional supplements (collectively, plant-based formulations) and synthetic cannabinoid formulations for oral delivery. By providing rapid onset and extended action, physiological benefits are observed earlier and last longer increasing the usefulness of these compounds.

The disclosed rapid onset and extended action plant-based formulations and synthetic cannabinoid formulations can create various administration benefits in providing therapeutically effective amounts in a variety of conditions. Exemplary administration benefits include increased absorption, increased bioavailability, faster onset of action, longer action, higher peak concentrations, faster time to peak concentrations, slower reduction in action, increased subjective therapeutic efficacy, and increased objective therapeutic efficacy.

The rapid onset nature of the plant-based formulations and synthetic cannabinoid formulations is created by including one or more N-acylated fatty amino acids, absorption enhancing agents, and/or various other beneficial carriers, such as surfactants, detergents, azones, pyrrolidones, glycols and bile salts in one portion of an oral formulation. In particular embodiments, N-acylated fatty amino acids can be linear, branched, cyclic, bicyclic, or aromatic including, for example, 1-50 carbon atoms in an oral formulation. That use of N-acylated fatty amino acids could provide a rapid onset benefit with a plant-based formulation or and synthetic cannabinoid formulation was unexpected given particular aspects of plant-based components described further herein. For example, the ability of N-acylated fatty amino acids to increase absorption of compounds is proportional to the water-solubility of a compound. Many plant-based compounds and synthetic cannabinoids are not water-soluble and would not have been expected to be affected by the presence of an N-acylated fatty amino acid.

The extended action nature of the plant-based formulations and synthetic cannabinoid formulations can be created by including one or more sustained release systems in a portion of an oral formulation. In particular embodiments, the sustained release system can include a release-controlling matrix, an enteric coating and/or a barrier layer.

In particular embodiments, the oral formulations can include a liquid component with a rapid onset profile and particles within the liquid component with an extended action profile. In particular embodiments, the particles can include a release-controlling matrix, an enteric coating and/or a barrier layer. These particles may additionally and optionally include a rapid onset shell.

In particular embodiments, the oral formulation can include a liquid component with rapid onset solid particles surrounding in an extended action profile liquid.

In particular embodiments, the oral formulations can include a tablet with a rapid onset shell and an extended action core. In particular embodiments, the extended action core can include a release-controlling matrix, an enteric coating and/or a barrier layer.

In particular embodiments, the plant-based formulations include Calophyllum brasiliense, Calophyllum caledonicurn, Calophyllum inophyllum, Calophyllum soulattri, Uncaria tomentosa, Thymus vulgaris, Matricaria recutita, Salix alba, Calendula officinalis, Usnea barbata, Ligusticum porterii-osha, Gaultheria procumbens, Camellia sinensis, Vaccinium myrtillus, Melissa officinalis, Allium sativum, Camellia sinensis, Krameria triandra, Punica granatum, Viburnum plicatum, Nicotiana tabacum, Duboisia hopwoodii, Asclepias syriaca, Curcumalonga, Cannabis sativa, Cannabis indica, Cannabis ruderalis, and Acer spp., or an extract thereof. In particular embodiments, the plant-based formulations include the cannabis plant, or an extract thereof.

Exemplary synthetic cannabinoids are described in the Detailed Description.

### BRIEF DESCRIPTION OF THE FIGURES

FIGS. 1A and 1B show an established correlation between water-solubility and the ability of sodium N-[8-(2-hydroxybenzoyl) amino] caprylate (SNAC) to improve a molecule's absorption. FIG. 1A shows the multiple of improvement from SNAC plotted for cromolyn, vitamin B12, atorvastatin, and ibandronate, along with the aqueous solubility of each molecule. The plotted data shows a striking fit to a logarithmic trendline (R²=0.998), indicating a logarithmic relationship between the aqueous solubility of each and the extent to which SNAC improves absorption. As the water solubility of the molecule increases, SNAC's ability to enhance its absorption also increases. FIG. 1B plots the aqueous solubility of heparin, acyclovir, rhGH, PTH, MT-II, GLP-1, calcitonin, yy peptide, and THC according to the logarithmic trendline derived from FIG. 1A.

FIG. 2 provides exemplary plant-based cannabinoid structures.

FIG. 3 provides active components of other plant-based herbal formulations.

FIG. 4 provides exemplary structures of cannabinoids that can be synthetically derived (THC, nabilone, CBD, 7-OH-CBD, CBDV, 7-OHCBDV, and formulas I-XVI).

FIG. 5 provides modified amino acids of compounds I-XXXV.

FIG. 6 provides fatty acid amino acids of formula (a), (b), (c), (d), (e), (f), (g), (h), (i), (j), (k), (I), (m), (n), (o), (p), (q), and (r), wherein R1 is an alkyl group including 5 to 19 carbon atoms, R2 is H (i.e. hydrogen) or CH₃ (i.e. methyl group), and R3 is H; or a salt or the free acid form thereof.

FIGS. 7A and 7B provide the average results of the study comparing onset and duration of action of orally administered cannabis/N-[8-(2-hydroxybenzoyl) amino] caprylate (SNAC, “test”) formulation and cannabis (without SNAC, “control”) formulation.

FIGS. 8A-8F provide the results for each individual participant in the study comparing onset and duration of action of orally administered cannabis/N-[8-(2-hydroxybenzoyl) amino] caprylate (SNAC, “test”) formulation and cannabis (without SNAC, “control”) formulation.

FIG. 9 shows a comparison of intensity, duration and onset of action of orally administered cannabis formulations with a high SNAC dose (200 mg, “high dose”), a low SNAC dose (100 mg, “low dose”) and no SNAC (“control”).

FIG. 10 shows intensity, duration and onset of action of cannabis formulated with SNAC administered orally (“PO”) compared to cannabis administered by inhalation (“INH”).

FIG. 11 shows THC and CBD Cmax and AUC following a single oral administration to rats.

FIG. 12 shows THC and CBD Cₘₐₓ (ng/ml) and AUC (hr*ng/mL) following a single oral administration to rats.

FIG. 13 shows intensity, duration and onset of action of orally administered cannabis/N-[8-(2-hydroxybenzoyl) amino] caprylic acid (NAC, “test”) formulation and cannabis (without NAC, “control”) formulation.

### DETAILED DESCRIPTION

Despite the numerous benefits associated with plant-based compounds and nutritional supplements and synthetic cannabinoid formulations, when administered in oral form, their onset of action can be slow, which can detract from their usefulness in some instances. For example, after oral administration, THC has an onset of action of up to 1.5 hours and a peak effect at 2-4 hours. The duration of effects is 4-6 hours. THC is almost completely absorbed (90-95%) after single oral doses. However, due to a combined effect of first pass hepatic metabolism and poor aqueous solubility (THC water solubility is 2.8 mg/L) only 4-20% of the administered dose reaches the systemic circulation. Therefore, oral consumption of cannabis is characterized by low bioavailability of cannabinoids, and slow onset of action. Thus, as this one example provides, there is room for improvement in the oral administration of plant-based compounds and nutritional supplements and synthetic cannabinoid formulations.

In addition to room for improvement in the time for onset to action, it can also be beneficial to provide extended action of plant-based compounds and nutritional supplements and synthetic cannabinoid formulations to extend the time of beneficial effects and reduce the need for repeat dosing.

The current disclosure provides rapid onset and extended action plant-based medicinal compounds and nutritional supplements (collectively, plant-based formulations) and synthetic cannabinoid formulations for oral delivery. By providing rapid onset, physiological benefits are observed earlier increasing the usefulness of these compounds. By providing extended action, physiological benefits can be maintained for a longer duration, reducing the need for repeat dosing.

The disclosed rapid onset and extended action plant-based formulations and synthetic cannabinoid formulations can create various administration benefits in providing therapeutically effective amounts in a variety of conditions. Exemplary administration benefits include increased absorption, increased bioavailability, faster onset of action, higher peak concentrations, faster time to peak concentrations, longer action, increased subjective therapeutic efficacy, and increased objective therapeutic efficacy.

The rapid onset nature of the plant-based formulations and synthetic cannabinoid formulations is created by including one or more N-acylated fatty amino acids, absorption enhancing agents, and/or various other beneficial carriers, such as surfactants, detergents, azones, pyrrolidones, glycols and bile salts in an oral formulation in one portion of an oral formulation. In particular embodiments, N-acylated fatty amino acids can be linear, branched, cyclic, bicyclic, or aromatic including, for example, 1-50 carbon atoms in an oral formulation. That use of N-acylated fatty amino acids could provide a rapid onset benefit with a plant-based formulation and synthetic cannabinoid formulation was unexpected given particular aspects of plant-based components and synthetic cannabinoids described further herein. For example, the ability of N-acylated fatty amino acids to increase absorption of compounds is proportional to the water-solubility of a compound. Many plant-based compounds and synthetic cannabinoids are not water-soluble and would not have been expected to be affected by the presence of an N-acylated fatty amino acid.

Molecules that have been shown to have improved absorption when co-administered with an N-acylated fatty amino acid (e.g., SNAC) include water-soluble molecules such as cromolyn, vitamin B12), atorvastatin, ibandronate, heparin, acyclovir, recombinant human growth hormone (rhGH), parathyroid hormone 1-34 (PTH 1-34), α-melanotropin (MT-II), GLP-1, calcitonin, and peptide yy.

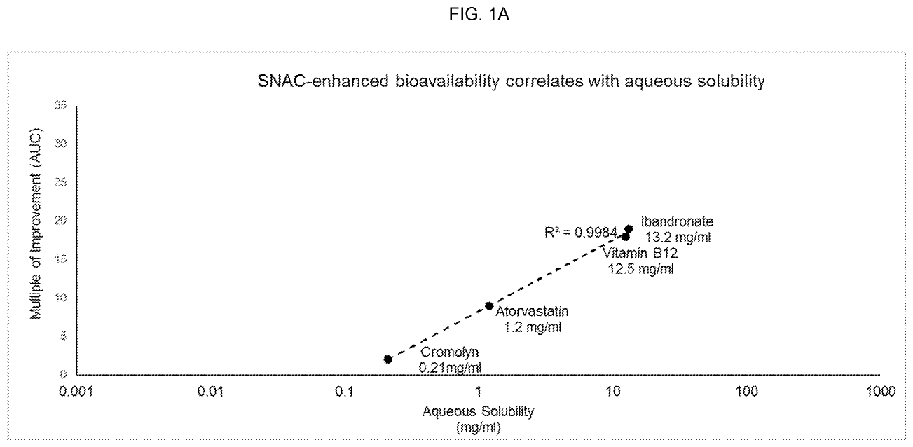

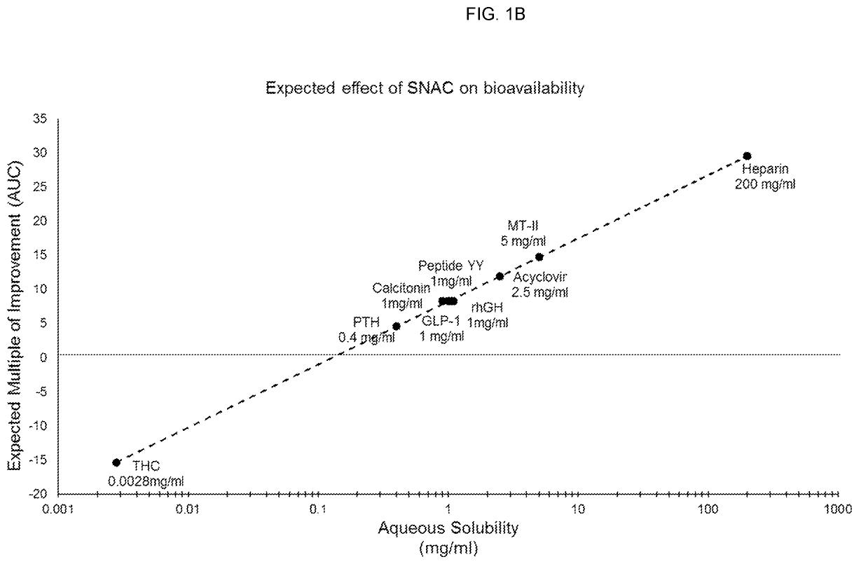

FIG. 1A shows an established correlation between water-solubility and the ability of SNAC to improve a molecule's absorption. For cromolyn, vitamin B12, atorvastatin, and ibandronate, published results include area under the curve (AUC), which is calculated from a time-course of plasma levels. To quantify the effect of co-administration with SNAC, a multiple of improvement can be calculated by dividing the AUC for a molecule with SNAC by the AUC for the molecule without SNAC. FIG. 1A shows the multiple of improvement from SNAC plotted for cromolyn, vitamin B12, atorvastatin, and ibandronate, along with the aqueous solubility of each molecule. The plotted data shows a striking fit to a logarithmic trendline (R²=0.998), indicating a logarithmic relationship between the aqueous solubility of each and the extent to which SNAC improves its absorption.

Heparin, acyclovir, rhGH, PTH, MT-II, GLP-1, calcitonin, and yy peptide are other molecules that have been shown to have SNAC-improved absorption, as demonstrated by Cmax (maximum drug plasma level) and/or Tmax (the time taken to reach maximum drug plasma level). As shown in FIG. 1B, each of these molecules has an aqueous solubility of more than 0.15 mg/ml, and therefore, the model accurately predicts that SNAC can improve their absorption. This result demonstrates that a SNAC-based absorption improvement correlates with a molecule's aqueous solubility. FIG. 1B further plots the aqueous solubility of THC (0.0028 mg/ml) to the logarithmic trendline and SNAC's predicted effect based on the same. Based at least on the foregoing, the results described herein were unexpected and would not have been reasonably expected by those of ordinary skill in the art.

The extended action nature of the plant-based formulations and synthetic cannabinoid formulations can be created by including one or more sustained release systems in a portion of an oral formulation. In particular embodiments, the sustained release system can include a release-controlling matrix, an enteric coating and/or a barrier layer.

In particular embodiments, the oral formulations can include a liquid component with a rapid onset profile and particles within the liquid component with an extended action profile. In particular embodiments, the particles can include a release-controlling matrix, an enteric coating and/or a barrier layer. These particles may additionally and optionally include a rapid onset shell.

In particular embodiments, the oral formulations can include a tablet with a rapid onset shell and an extended action core. In particular embodiments, the extended action core can include a release-controlling matrix, an enteric coating and/or a barrier layer.

Aspects of the disclosure are now described in more detail.

The current disclosure provides rapid onset and extended action oral formulations including (i) (a) a plant-based formulation including vegetable matter and/or (b) a synthetic cannabinoid and (ii) (a) a rapid onset carrier and (b) an extended action component. Plant-based formulations refer to plant-based medicinal compounds and plant-based nutritional supplements.

As indicated, embodiments disclosed herein include a rapid onset component and an extended action component. There are many approaches that can be used to create an oral formulation with a rapid onset component and an extended action component.

In particular embodiments, two individual oral formulations may be separately prepared and packaged for ingestion within a short time window of the other. In particular embodiments, the rapid onset component is provided as a first liquid formulation and the extended action component is provided as a second liquid formulation. In particular embodiments, the rapid onset component is provided as a liquid formulation and the extended action component is provided as a tablet. In particular embodiments, the rapid onset component is provided as a tablet and the extended action component is provided as a liquid. In particular embodiments, the rapid onset component is provided as a first tablet and the extended action component is provided as a second tablet.

In particular embodiments, one oral formulation can include the rapid onset component and the extended action component. These embodiments can include, for example, a tablet with a rapid onset shell and an extended action core that is a solid or a liquid. Tablets with a rapid onset shell and an extended action core can have one or more coatings (e.g., overcoats and/or enteric coatings) or layers (e.g., barrier layers) providing different release profiles. Particular embodiments include a tablet with an extended action core and a compressed outer rapid onset coating. Particular embodiments include a double layered tablet or a multiple-layered tablet.

In particular embodiments, an oral formulation including the rapid onset component and the extended action component can include a liquid providing the rapid onset component and particles within the liquid providing the extended action component. In these embodiments, the particles may be engineered to provide a desired extended action release profile.

In particular embodiments, an oral formulation including the rapid onset component and the extended action component can include a solid providing the rapid onset component and a liquid contained within the solid providing the extended action component. In these embodiments, the liquid may be engineered to provide a desired extended action release profile.

Plant-based medicinal compounds and synthetic cannabinoid formulations can provide therapeutically-effective amounts to treat a condition, such as those described in the Background of the Disclosure. Plant-based nutritional supplements and synthetic cannabinoid formulations can claim a benefit related to a classical nutrient deficiency; describe how the supplement is intended to affect the structure or function of the human body; characterize a documented mechanism by which the supplement acts to maintain such structure or function; and/or describe general well-being associated with consumption of the product. In particular embodiments, a nutritional supplement and/or synthetic cannabinoid formulation may not claim to diagnose, mitigate, treat, cure, or prevent a specific disease or class of diseases.

Plant-based formulations include vegetable matter. Vegetable matter is matter produced by a plant and includes any whole plant or plant part (e.g., bark, wood, leaves, stems, roots, flowers, fruits, seeds, or parts thereof) and/or exudates or extracts thereof. In particular embodiments, plant-based formulations include botanical products. Botanical products can include plant materials, algae, macroscopic fungi, and/or combinations thereof. In particular embodiments, plant-based formulations include a mixture of various types of vegetable matter. Plant-based formulations can also include materials derived from vegetable matter including resins, oils (e.g., essential oils), spices, dried flowers, kief, tinctures, infusions, etc. In particular embodiments, the vegetable matter has little or no water solubility. In particular embodiments, plant-based formulations do not include synthetic, semi-synthetic, or chemically-modified drugs.

In particular embodiments, the plant-based formulations include vegetable matter derived from Calophyllum brasiliense, Calophyllum caledonicurn, Calophyllum inophyllum, Calophyllum soulattri, Uncaria tomentosa, Thymus vulgaris, Matricaria recutita, Salix alba, Calendula officinalis, Usnea barbata, Ligusticum porterii-osha, Gaultheria procumbens, Camellia sinensis, Vaccinium myrtillus, Melissa officinalis, Allium sativum, Camellia sinensis, Krameria triandra, Punica granatum, Viburnum plicatum, Nicotiana tabacum, Duboisia hopwoodii, Asclepias syriaca, Curcumalonga, Hypericum perforatum, Polygonum cuspidatum, Vitis spp, Camellia sinensis, Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., Mentha spp., Cannabis sativa, Cannabis indica, Cannabis ruderalis and/or Acer spp. or an extract thereof.

In particular embodiments, the plant-based formulations include vegetable matter derived from the cannabis plant. The cannabis plant refers to a flowering plant including the species (or sub-species) Cannabis sativa, Cannabis ruderalis, and Cannabis indica.

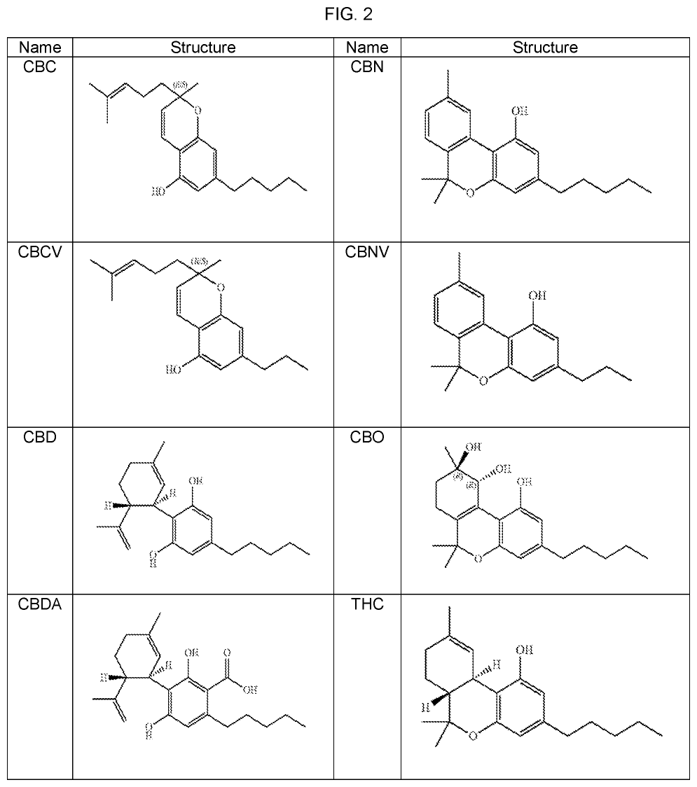

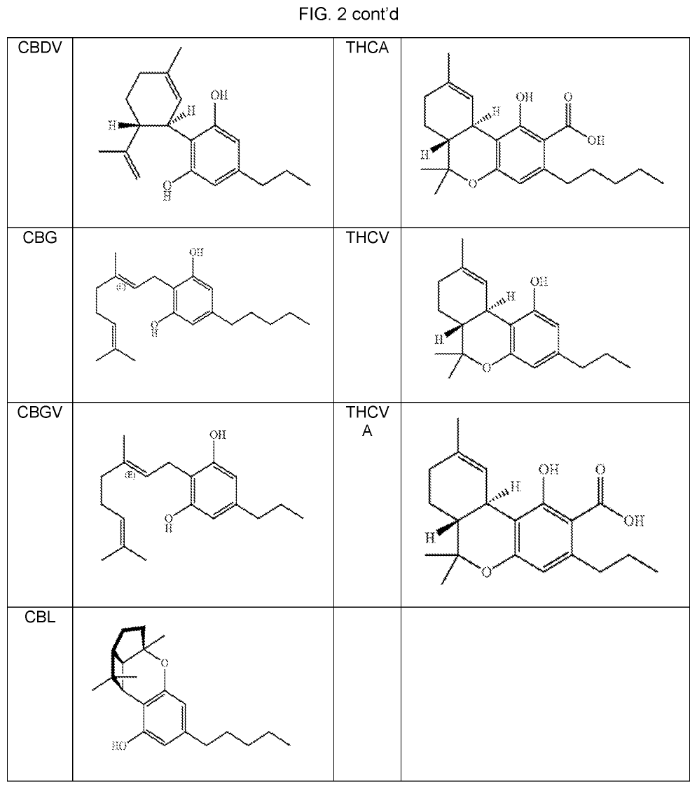

Particular extracts of the cannabis plant include cannabinoids. Cannabinoids are a group of cyclic molecules from cannabis plants that activate cannabinoid receptors (i.e., CB1 and CB2) in cells. There are at least 85 different cannabinoids that can be isolated from cannabis. Many cannabinoids produced by cannabis plants, such as Δ9-Tetrahydrocannabinol (THC) and cannabidiol (CBD), have very low or no solubility in water. The most notable cannabinoids are THC and CBD. Additional examples include cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and tetrahydrocannabivarinic acid (THCVA). See, for example, FIG. 2. Extracts of the cannabis plant similarly include flavonoid compounds, terpenes, terpenoid, and synthetic, semisynthetic or highly purified versions of any such constituent.

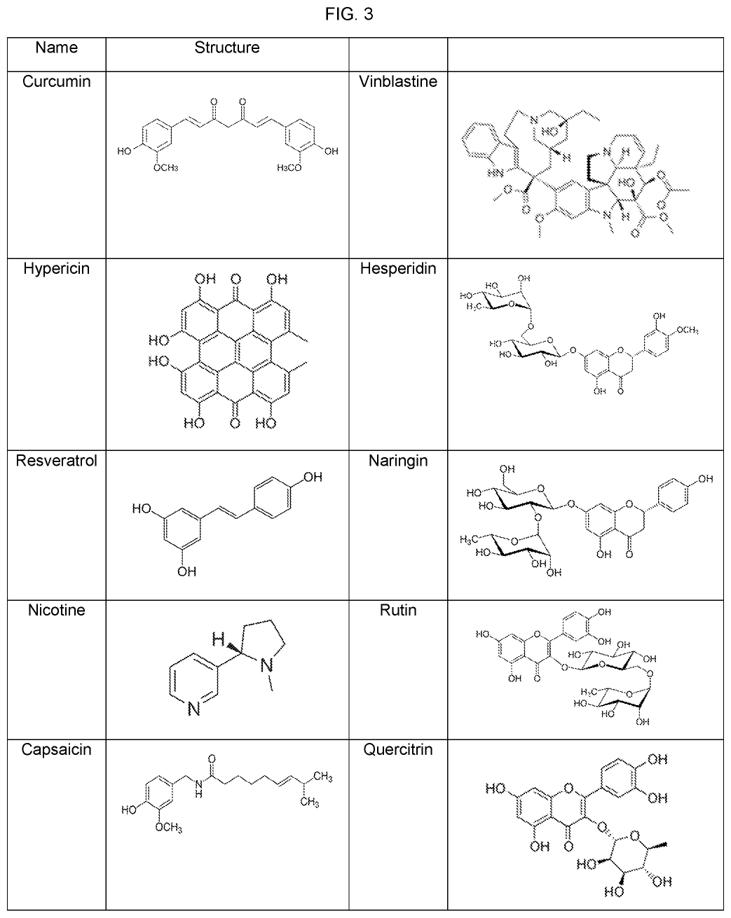

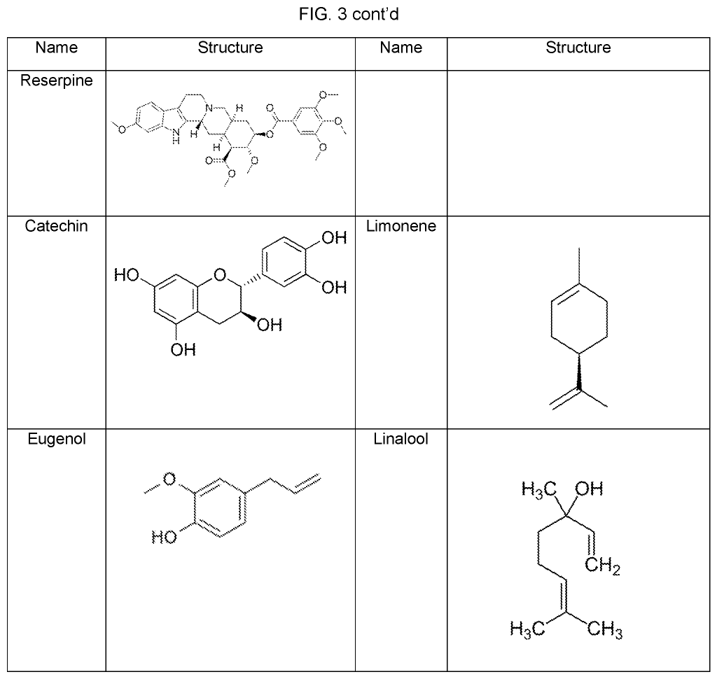

In particular embodiments the oral formulations include curcumin. Curcumin is a phenolic compound with poor water solubility (<0.1 mg/ml) that is responsible for the yellow color of turmeric, a spice derived from Curcuma longa. Curcumin has been shown to have anti-inflammatory and anti-cancer effects, and may be useful for treating chronic inflammatory bowel disease, chronic hepatitis, chronic bronchial asthma, and psoriasis. For the chemical structure of curcumin, see, for example, FIG. 3.

In particular embodiments the oral formulations include hypericin. Hypericin is a napthodianthrone that is insoluble in water, and is a main active component of Hypericum perforatum, or St. John's wort. Hypericin is used as an anti-depressant, and can also be used for photodynamic cancer therapy because it preferentially accumulates in cancer tissue and causes photosensitivity. For the chemical structure of hypericin, see, for example, FIG. 3.

In particular embodiments the oral formulations include resveratrol. Resveratrol is a main active component of Polygonum cuspidatum and can also be found in the skins of many other plants. For example, resveratrol is also found in the skin of grapes (i.e., plants of the genus Vitis, also referred to as Vitis spp.), blueberries, raspberries, and mulberries. Resveratrol has poor water solubility (0.03 mg/ml), and is a potent antioxidant. For the chemical structure of resveratrol, see, for example, FIG. 3.

In particular embodiments the oral formulations include nicotine. Nicotine is a main active component of tobacco, a product prepared from leaves of Nicotiana plants, such as Nicotiana tabacum. Oral formulations including nicotine can be useful, for example, as nicotine replacement therapy to promote smoking cessation. For the chemical structure of nicotine, see, for example, FIG. 3.

In particular embodiments the herbal compositions include catechin. Catechin is a polyphenol with low water solubility, and includes four isomers: (−)-epicatechin, (+)-epicatechin, (−)-catechin, and (+)-catechin. Catechin is found in many plants, and dietary sources of catechin include tea (Camellia sinsensis), cocoa (Theobroma cacao), açai, apple, and pear. Catechin is a potent antioxidant and has anti-inflammatory effects. For the chemical structure of catechin, see, for example, FIG. 3.

In particular embodiments the herbal compositions include capsaicin. Capsaicin is an alkaloid with low water solubility that can be used as an analgesic, and for treating neuralgia. Capsaicin is present in the fruits of plants of the genus Capsicum, such as Capsicum annuum, Capsicum chinense, capsicum baccatum, and Capsicum pubescens. For the chemical structure of capsaicin, see, for example, FIG. 3.

In particular embodiments the herbal compositions include reserpine. Reserpine is an indole alkaloid with low water solubility, and is found in the dried roots of plants of the genus Rauwolfia, such as Rauwolfia vomitoria and Rauwolfia serpentina. Reserpine-containing extracts have been used in India for centuries, for treating insanity, fevers, and snakebites. Reserpine is also used to treat hypertension. For the chemical structure of reserpine, see, for example, FIG. 3.

In particular embodiments the herbal compositions include vinblastine. Vinblastine is an alkaloid with low water solubility, and is produced by plants of the genus Vinca, such as Vinca rosea. Vinblastine can block cellular division by disrupting microtubule formation and is used as a chemotherapy agent to treat a variety of cancers. For the chemical structure of vinblastine, see, for example, FIG. 3.

In particular embodiments the herbal compositions include hesperidin. Hesperidin is a glycoside with low water solubility. Hesperidin is found in the fruit of citrus trees, such as Citrus aurantium, Citrus sinensis, Citrus limon, and Citrus aurantifolia. Hesperidin is an antioxidant, is anti-inflammatory, may help prevent cancer, and is used to treat blood vessel conditions such as hemmorhoids, variscose veins and poor circulation. For the chemical structure of hesperidin, see, for example, FIG. 3.

In particular embodiments the herbal compositions include naringin. Naringin is a glycoside with low water solubility that is present in the fruits of Citrus plants, such as Citrus sinensis, Citrus aurantium, Citrus reticulate, Citrus clementina, and Citrus bergamia. Naringin is an antioxidant, is anti-inflammatory, and can improve glucose regulation. For the chemical structure of naringin, see, for example, FIG. 3.

In particular embodiments the herbal compositions include rutin. Rutin is a glycoside with low water solubility, and is found in buckwheat (Fagopyrum tataricum), Rheum species (such as Rheum rhabarbarum, or rhubarb), and asparagus. Rutin is a potent antioxidant. For the chemical structure of rutin, see, for example, FIG. 3.

In particular embodiments the herbal compositions include quercitrin. Quercitrin is a glycoside with low water solubility, and is formed by the flavonoid quercetin and the deoxy sugar rhamnose. Quercitrin has potent antioxidant properties and is found in a wide variety of plants, such as buckwheat (Fagopyrum tataricum) and St. John's wort (Hypericum perforatum). For the chemical structure of quercitrin, see, for example, FIG. 3.

In particular embodiments the herbal compositions include eugenol. Eugenol is a terpene with low water solubility and has anti-inflammatory effects. Eugenol is found in clove (Syzygium aromaticum) oil, cinnamon, nutmeg, cannabis, and bay leaf. For the chemical structure of eugenol, see, for example, FIG. 3.

In particular embodiments the herbal compositions include limonene. Limonene is a terpene with low water solubility, which forms two isomers. The D-isomer of limonene has a potent orange aroma and can be found in high quantities in Citrus fruits, whereas the L-isomer has a piney aroma and is common in oils extracted from mint (genus Mentha). Limonene is used for weight loss, cancer prevention, to treat bronchitis, and to help control cholesterol levels. For the chemical structure of limonene, see, for example, FIG. 3.

In particular embodiments the herbal compositions include linalool. Linalool is a terpene with low water solubility, which forms two enantiomers known as licareol and coriandrol. Linalool is produced in high quantities by lavender (genus Lavandula), and is produced by many other plants such as birch trees, mint, citrus fruits, and cinnamon. Linalool has sedative, anti-inflammatory, and anxiolytic properties. For the chemical structure of linalool, see, for example, FIG. 3.

Components of plant-based formulations can be produced by, e.g., pulverization, decoction, expression, and extraction of a starting plant product. The term “extract” can include all of the many types of preparations containing some or all of the active ingredients found in the relevant plants. Extracts may be produced by cold extraction techniques using a variety of different extraction solvents including water, fatty solvents (such as olive oil), and alcoholic solvents (e.g. 70% ethanol). Cold extraction techniques are typically applied to softer parts of the plant such as leaves and flowers, or in cases wherein the desired active components of the plant are heat labile. Alternatively, the aforementioned solvents may be used to produce extracts of the desired plants by a hot extraction technique, wherein said solvents are heated to a high temperature, the precise value of said temperature being dependent on the properties of the chosen solvent, and maintained at that temperature throughout the extraction process. Hot extraction techniques are more commonly applied to the harder, tougher parts of the plant, such as bark, woody branches and larger roots. In some cases, sequential extractions can be performed in more than one solvent, and at different temperatures. The plant extract may be used in a concentrated form. Alternatively, the extract may be diluted as appropriate to its intended use.

WO2004/026857 provides a method for preparing a purified cannabis extract, wherein the cannabinoids are purified to at least 99% wt % THC (Δ9-tetrahydrocannabinol). In this method a crude ethanolic extract of Cannabis plant material is passed through a column of activated charcoal and evaporated by means of rotary evaporation. The resulting THC enriched extract is subsequently passed through a column packed with Sephadex LH20 and eluted with chloroform/dichloromethane. The solvent used is removed by means of rotary evaporation. In order to further increase the purity of the THC enriched extract, the extract is dissolved in methanol and subsequently in pentane and subjected to rotary evaporation twice.

US2015/0126754 describes a) providing a crude solvent extract of Cannabis plant material; b) subjecting the crude extract to thin film evaporation to obtain a refined extract; c) chromatographically fractionating the refined extract to produce one or more high purity fractions having a THC content higher than a preset value and one or more low purity fractions having a THC content lower than the preset value, wherein the preset value is in the range of 95-99% by weight of dry matter; d) subjecting the one or more high purity fractions to another thin film evaporation; and e) collecting a THC isolate containing at least 97% THC by weight of dry matter; and wherein in step b) and/or in step d) the thin film evaporation is carried out by using wiped film evaporation. This method offers the advantage that it yields a high purity THC extract in good yield and without using solvents that pose a health risk. The method further offers the advantage that it is highly reproducible in that it produces THC-isolate with a specific cannabinoid profile. More particularly, the method yields a THC isolate that contains at least 97.0-99.5% THC and 0.4-2.0% of other cannabinoids, including at least 0.3% Cannabinol and Cannabidiol (all percentages by weight of dry matter).

Additional procedures for producing plant extracts (including hot extraction, cold extraction and other techniques) are described in publications including “Medicinal plants: a field guide to the medicinal plants of the Land of Israel (in Hebrew), author: N. Krispil, Har Gilo, Israel, 1986” and “Making plant medicine, author: R. Cech, pub. by Horizon Herbs, 2000”.

In particular embodiments, plant components of plant-based formulations (e.g., plant extracts) may be sterilized, for example by autoclaving, and then allowed to cool and stored at an appropriate temperature (e.g., −20° C.). In particular embodiments, further purification to a molecular weight cut-off (e.g., below 10,000 Da) can be carried out, for example, by membrane ultrafiltration before storage.

Synthetic cannabinoids include cannabinoids that are chemically produced. Synthetic cannabinoids also include cannabinoids that are found in nature, but are produced chemically. Synthetic cannabinoids also include chemically produced derivatives and analogs of cannabinoids.

The term “derivative” in chemistry refers to a compound that is obtained from a similar compound or a precursor compound by a chemical reaction.

The term “analog” (also “structural analog” or “chemical analog”) is used to refer to a compound that is structurally similar to another compound but differs with respect to a certain component, such as an atom, a functional group, or a substructure.

As indicated, examples of cannabinoids derived from plants include CBG, CBC, CBD, CBN, THC, iso-THC, CBE, CBL, CBDV, THCV, and CBT. In particular embodiments, synthetic cannabinoids include natural cannabinoids that are synthesized chemically and also their analogs and derivatives. Derivatives of natural cannabinoids can include metabolites of cannabinoids which are disclosed in WO 2015/198078. For example, the metabolite of CBD includes 7-OH-CBD and the metabolite of CBDV includes 7-OH-CBDV.

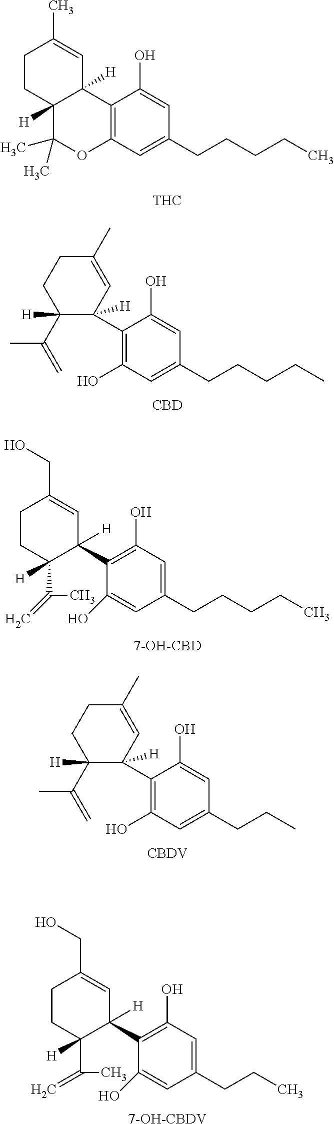

Other examples of synthetic cannabinoids include 3-carbamoyl-2-pyridone, and its derivatives and/or analogs disclosed in US 2008/0103139; pyrimidine derivatives and/or analogs disclosed in US 2006/0293354; carenadiol and its derivatives and/or analogs thereof disclosed in U.S. Pat. No. 4,758,597; cannabinoid carboxylic acids and their derivatives and/or analogs disclosed in WO 2013/045115; pyrido[3,2-E][1,2,4]triazolo[4,3-C]pyrimidine and its derivatives and/or analogs disclosed in WO 2008/118414; tetrahydro-pyrazolo[3,4-C] pyridine and its derivatives and/or analogs disclosed in WO 2007/112399; bicyclo[3.1.1]heptan-2-one cannabinoid and its derivatives and/or analogs disclosed in WO 2006/043260; resorcinol and its derivatives and/or analogs disclosed in WO 2005/0123051; dexanabinol compounds and their derivatives and/or analogs disclosed in WO 2004/050011; cannabimimetic lipid amide compounds and their derivatives and/or analogs disclosed in WO 2000/032200; nabilone and its derivatives and/or analogs disclosed in US 2010/0168066; 2-oxoquinolone compounds and their derivatives and/or analogs disclosed in US 2003/0191069; and 3,4-diaryl-4,5-dihydro-(h)-pyrazole-1-carboxamide and its derivatives and/or analogs disclosed in US 2011/0137040.

In particular embodiments, 3-carbamoyl-2-pyridone and its derivatives and/or analogs include methyl 3-methyl-2-{[2-oxo-1-(2-oxo-ethyl)-1,2,5,6,7,8,9,10-octahydro-cycloocta[b]pyridine-3-carbonyl]-amino}butyrate; dimethyl 2-[(1-cyclohexylmethyl-5,6-dimethyl-2-oxo-1,2-dihydropyridine-3-carbonyl)-amino]-succinate; and methyl 2-{[1-(3-methoxycarbonyamino-propyl)-2-oxo-1,2,5,6,7,8,9,10-octahydro-cycloocta[b]pyridine-3-carbonyl]-amino}-2-methyl-propionate.

In particular embodiments, pyrimidine derivatives and/or analogs include a compound having Formula (I) (2-((2,4-dichlorophenyl)amino)-N-((tetrahydro-2H-pyran-4-yl)methyl)-4-(trifluoromethyl)pyrimidine-5-carboxamide),

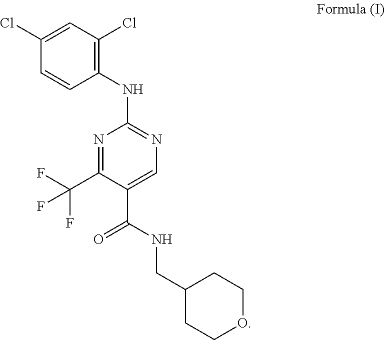 [SMILES: O=C(NCC1CCOCC1)c1cnc(Nc2ccc(Cl)cc2Cl)nc1C(F)(F)F]

Other pyrimidine derivatives and/or analogs include 2-(3-Chlorophenylamino)-4-trifluoromethylpyrimidine-5-carboxylic acid cyclohexylmethyl-amide; 2-Phenylamino-4-trifluoromethylpyrimidine-5-carboxylic acid cyclohexylmethyl-amide; 1-[2-(2,3-Dichlorophenylamino)-4-trifluoromethylpyrimidin-5-yl]-1-morphol-in-4-yl-methanone; 1-[2-(2,4-Dichlorophenylamino)-4-trifluoromethylpyrimidin-5-yl]-1-morphol-in-4-yl-methanone; and 2-(3-Chlorophenylamino)-4-trifluoromethylpyrimidin-5-carboxylic acid cyclopentylamide.

In particular embodiments, carenadiol and its derivatives and/or analogs include compounds having Formula (II),

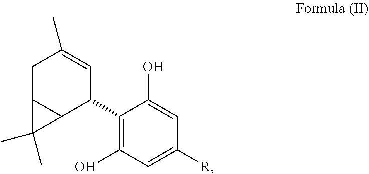

wherein R is a lower alkyl having 1 to 9 carbon atoms including isomeric forms such as i-butyl, n-butyl, and t-butyl. In particular embodiments, R is C₅H₁₁ or 1,1-dimethylheptyl.

In particular embodiments, cannabinoid carboxylic acids and their derivatives and/or analogs include compounds having Formula (III), (IV), (V), or (VI),

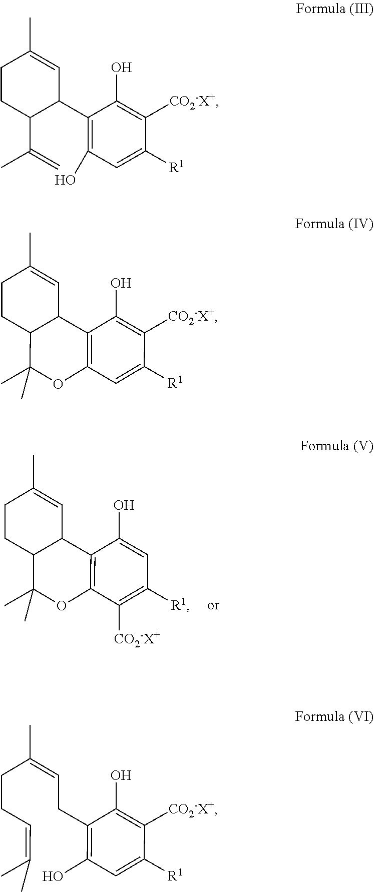

wherein:
R¹ is a straight-chain, branched or cyclic hydrocarbon residue with one C atom to 12 C atoms; and
X⁺ is NH₄⁺, mono-, di- or trivalent metal ions; or primary, secondary, tertiary or quaternary organic ammonium ions with up to 48 C atoms, which may bear still further functional groups.

Examples of multivalent ammonium ions include N,N-dicyclo-hexylamine-H+ and N,N-dicyclohexyl-N-ethylamine-H⁺, X⁺ can also be the hydrogen cation of a pharmaceutical active substance with at least one basic nitrogen atom, such as for example morphine, methadone (or an enantiomer thereof) or hydromorphone.

In particular embodiments, pyrido[3,2-E][1,2,4]triazolo[4,3-C]pyrimidine and its derivatives and/or analogs include 5-tert-butyl-8-(2-chlorophenyl)-9-(4-chlorophenyl)pyrido[3,2-e][1,2,4]triazolo[4,3-c]pyri midi n-3(2H)-one; 8-(4-bromo-2-chlorophenyl)-5-tert-butyl-9-(4-chlorophenyl)pyrido[3,2-e][1,2,4]triazolo[4,3-c]pyrimidin-3(2H)-one; 5-tert-butyl-9-(4-chlorophenyl)-8-(2-methylphenyl)pyrido[3,2-e][1,2,4]triazolo[4,3-c]pyrimidin-3(2H)-one; 9-(4-bromophenyI)-5-tert-butyl-8-(2-chlorophenyl)pyrido[3,2-e][1,2,4]triazolo[4,3-c]pyrimidin-3(2H)-one; and 5-tert-butyl-8-(2-chlorophenyl)-9-(4-chlorophenyl)pyrido[3,2-e][1,2,4]triazolo[4,3-c]pyrimidine.

In particular embodiments, tetrahydro-pyrazolo[3,4-C] pyridine and its analogs and/or derivatives include compounds having Formula (VII), (VIII), (IX), (X), or (XI),

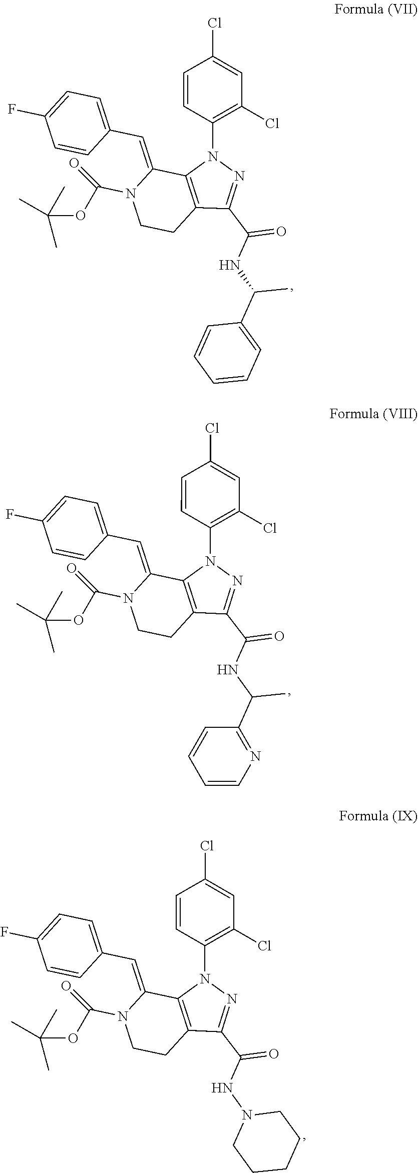

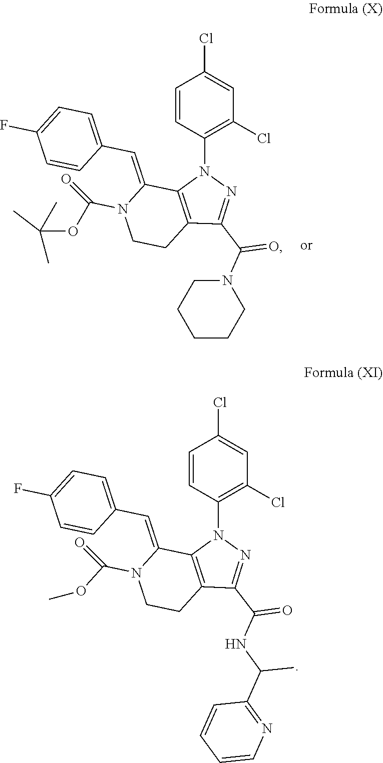 [SMILES: CC(C)(C)OC(=O)N1CCc2c(C(=O)N3CCCCC3)nn(-c3ccc(Cl)cc3Cl)c2/C1=C/c1ccc(F)cc1 ; COC(=O)N1CCc2c(C(=O)NC(C)c3ccccn3)nn(-c3ccc(Cl)cc3Cl)c2/C1=C/c1ccc(F)cc1]

In particular embodiments, bicyclo[3.1.1]heptan-2-one cannabinoids and their derivatives and/or analogs include compounds having Formula (XII),

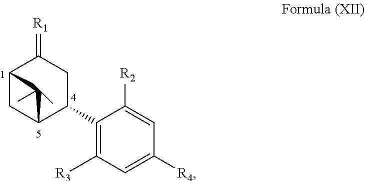

having a specific stereochemistry wherein C-4 is S, the protons at C-1 and C-5 are cis in relation to one another and the protons at C-4 and C-5 are trans; and wherein:
R1 is (a) 0 or S; (b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ or C₁-C₆alkyl-N(R″)₂ wherein at each occurrence R″ is independently selected from the group consisting of hydrogen, C(O)R′″, C(O)N(R′″)₂, C(S)R″, saturated or unsaturated, linear or branched C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂alkyl; or (c) NR″ or N—OR″ wherein R″ is as previously defined;
R₂ and R₃ are each independently (a) —R″, —OR″, —N(R″)₂, —SR″, —S(O)(0)NR″, wherein at each occurrence R″ is as previously defined; (b) —S(O)R^{b}, —S(O)(O)R^{b} wherein R^{b} is selected from the group consisting of hydrogen, saturated or unsaturated, linear or branched C₁-C₆alkyl, C₁-C₆alkyl-OR″, and C₁-C₆alkyl-N(R″)₂, wherein R″ is as previously defined; or (c) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear or branched C₁-C₆ alkyl and R^{e} is at each occurrence selected from the group consisting of hydrogen and R^{d} as previously defined; and
R₄ is (a) R wherein R is selected from the group consisting of hydrogen, halogen, OR″, OC(O)R′″, C(O)OR′″, C(O)R′″, OC(O)OR′″, CN, N(R′″)₂, NC(O)R′″, NC(O)OR′″, C(O)N(R′″)₂, NC(O)N(R′″)₂, and SR″, wherein at each occurrence R′″ is as previously defined; (b) a saturated or unsaturated, linear, branched or cyclic C₁-C₁₂ alkyl-R wherein R is as previously defined; (c) an aromatic ring which can be further substituted at any position by R wherein R is as previously defined; or (d) a saturated or unsaturated, linear, branched or cyclic C₁-C₁₂ alkyl optionally terminated by an aromatic ring which can be further substituted as defined in (c).

In particular embodiments, resorcinol and its derivatives and/or analogs include compounds having Formula (XIII),

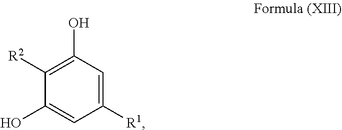

wherein:
R¹ is (a) straight or branched alkyl chain of 7 to 12 carbon atoms; (b) —O—R³, where R³ is a straight or branched alkyl chain of 5 to 9 carbon atoms, optionally substituted by one phenyl group; or (c) —(CH₂)ₙ—O—R⁴, where n is an integer from 1 to 7, and R⁴ is a straight alkyl chain of 1 to 5 carbon atoms; and
R² is a non-cyclic terpenoid comprising from 10 to 30 carbon atoms.

In particular embodiments, resorcinol and its derivatives and/or analogs include compounds having Formula (XIII), wherein R¹ and R² are as follows:
R¹ is a straight alkyl chain of 5 to 8 carbon atoms, optionally substituted with one methyl group; and
R² is selected from geranyl optionally substituted with one —OH, and farnesyl optionally substituted with one —OH.

In particular embodiments, resorcinol and its derivatives and/or analogs include compounds having Formula (XIII), wherein:
R¹ is (a) straight or branched alkyl chain of 7 to 12 carbon atoms; (b) —O—R³, where R³ is a straight or branched alkyl chain of 5 to 9 carbon atoms, optionally substituted by one phenyl group; or (c) —(CH₂)ₙ—O—R⁴, where n is an integer from 1 to 7, and R⁴ is a straight alkyl chain of 1 to 5 carbon atoms; and
R² is a non-cyclic terpenoid comprising from 10 to 30 carbon atoms; with the proviso that when R¹ is isononyl, R² is not geranyl.

In particular embodiments, resorcinol and its derivatives and/or analogs include compounds having Formula (XIII), wherein R¹ is (a) a straight or branched alkyl of 7 to 12 carbon atoms; (b) a group —O—R³, where R³ is a straight or branched alkyl of 5 to 9 carbon atoms, or a straight or branched alkyl substituted at the terminal carbon atom by a phenyl group; or (c) a group —(CH₂)ₙ—O-alkyl, where n is an integer from 1 to 7 and the alkyl group contains 1 to 5 carbon atoms.

In particular embodiments, resorcinol and its derivatives and/or analogs include compounds of Formula (XIII), wherein R² is a non-cyclic terpenoid carbon chain such as geranyl, farnesyl, and related non-cyclic terpenes and their isomers as well as other non-cyclic paraffinic or olefinic carbon chains.

In particular embodiments, resorcinol and its derivatives and/or analogs include compounds of Formula (XIII), wherein R¹ is dimethylheptyl and R² is geranyl.

In particular embodiments, dexanabinol compounds and their derivatives and/or analogs include high enantiomeric purity compounds having Formula (XIV),

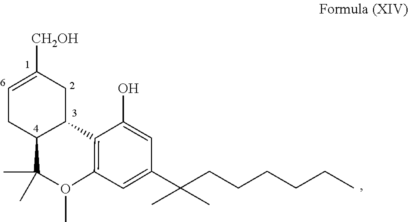

and having the (3S, 4S) configuration and being in enantiomeric excess of at least 99.90% over the (3R,4R) enantiomer.

In particular embodiments, cannabimimetic lipid amide compounds and their derivatives and/or analogs include compounds having Formula (XV),

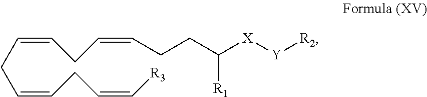

wherein:
X is one of the group consisting of C═O and NH, and Y is the other of that group. Expressed another way, X may be C═O and Y may be NH, or Y may be C═O and X may be NH, but both X and Y may not be the same group.
R₁ is H or an alkyl group. In particular embodiments, R₁ is H, CH₃, or (CH₃)₂;
R₂ is an alkyl, a substituted alkyl, an alkenyl or an alkynyl group. In particular embodiments, R₂ is CH(R) CH₂Z, CH₂CH(R)Z, or CH(R)(CH₂)ₙCH₂Z; R being H, CH, CH₃, CHCH, CH₂CF₃, or (CH₃)₂, Z being H, halogen, N₃, NCS, or OH; and n being selected from the group consisting of 0, 1 and 2.
R₃ is an alkyl, a substituted alkyl, an aryl, an alkylaryl, an O-alkyl, an O-alkylaryl, a cyclic and a heterocyclic group. O-alkyl and O-alkylaryl refer to groups in which an oxygen atom is interposed between carbon atoms on the anandamide portion and substituent group. Examples of such R₃ groups include cyclohexyl, cyclopentyl, alkylcyclohexyl, alkylcyclopentyl, piperidinyl, morpholinyl and pyridinyl. In particular embodiments, R₃ is n-C₅H₁₀Z′, n-C₇H₁₄Z′, or 1′,1′-C(CH₃)₂(CH₂)₅ CH₂Z′; Z′ being H, halogens, CN, N₃, NCS, or OH.

In particular embodiments, cannabimimetic lipid amide compounds and their derivatives and/or analogs include compounds having Formula (XVI),

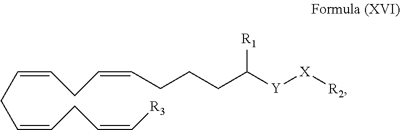

wherein:
Y is one of the group consisting of C═O and NH and X is the other of that group.
R₁ is H or an alkyl group. In particular embodiments, R₁ is H, CH₃, or (CH₃)₂.
R₂ is an alkyl, a substituted alkyl, an alkenyl, an alkynyl, an O-alkyl, a cyclic, a polycyclic, or a heterocyclic group. In particular embodiments, R₂ is

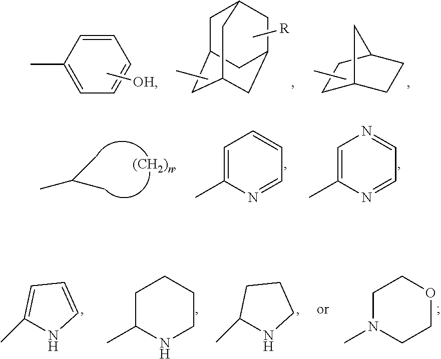

CH═CH₂, CH═C(CH₃)₂, CECH, CH₂OCH₃, CH(R)(CH₂)ₙCH₂Z, or CH₂CH(R)(CH₂)ₙZ; R being H, CH₃ or (CH₃)₂, Z being H, halogens, N₃, NCS, OH, or OAc; and n 0, 1, or 2; and R₃ is an alkyl, a substituted alkyl, an aryl, an alkylaryl, an O-alkyl, an O-alkylaryl, a cyclic, or a heterocyclic group. In particular embodiments R₃ includes cyclohexyl, cyclopentyl, alkylcyclohexyl, alkylcyclopentyl, piperidinyl, morpholinyl and pyridinyl. In particular embodiments, R₃ is n-C₅H₁₀Z′, n-C₆H₁₂Z, n-C₇H₁₄Z′, or 1′,1′-C(CH₃)₂CH₂)₅ CH₂Z′; Z′ being H, halogen, CN, N₃, NCS, or OH.

In particular embodiments, nabilone and its derivatives and/or analogs include compounds having Formula (XVII):

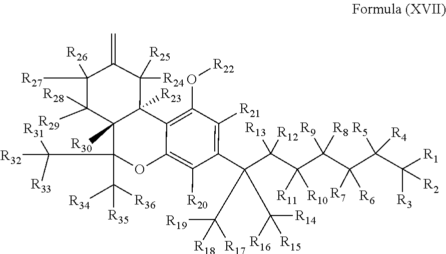

wherein:

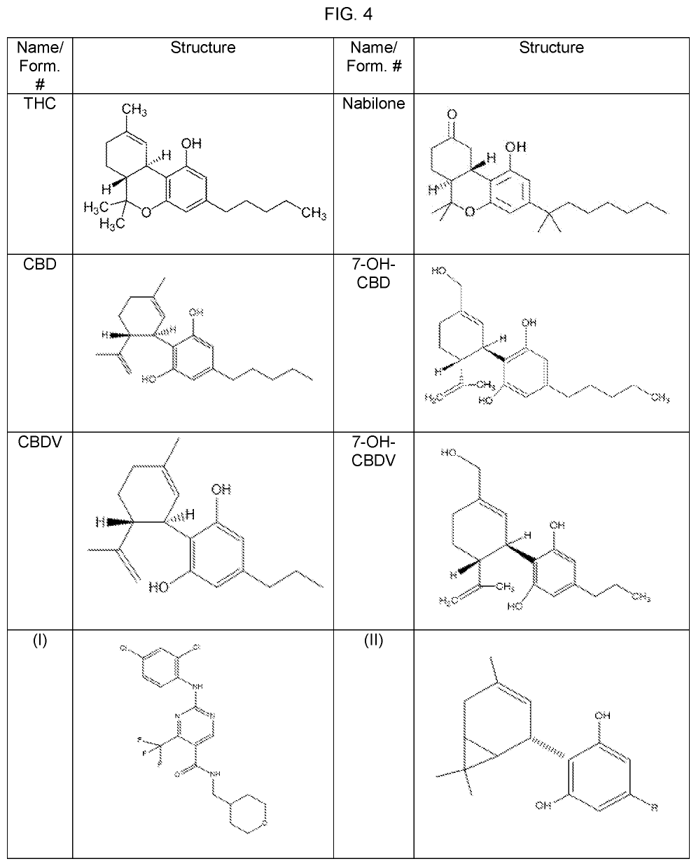

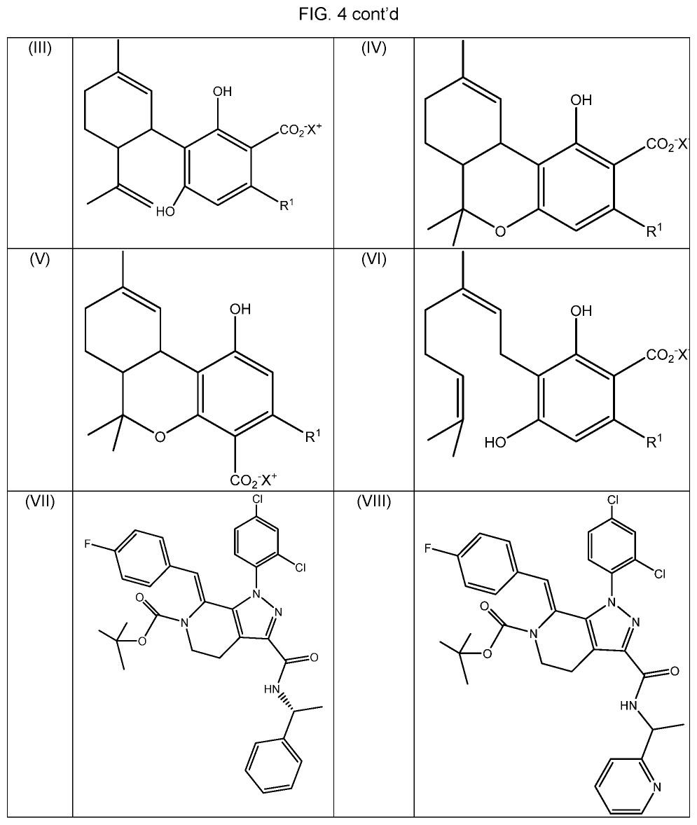

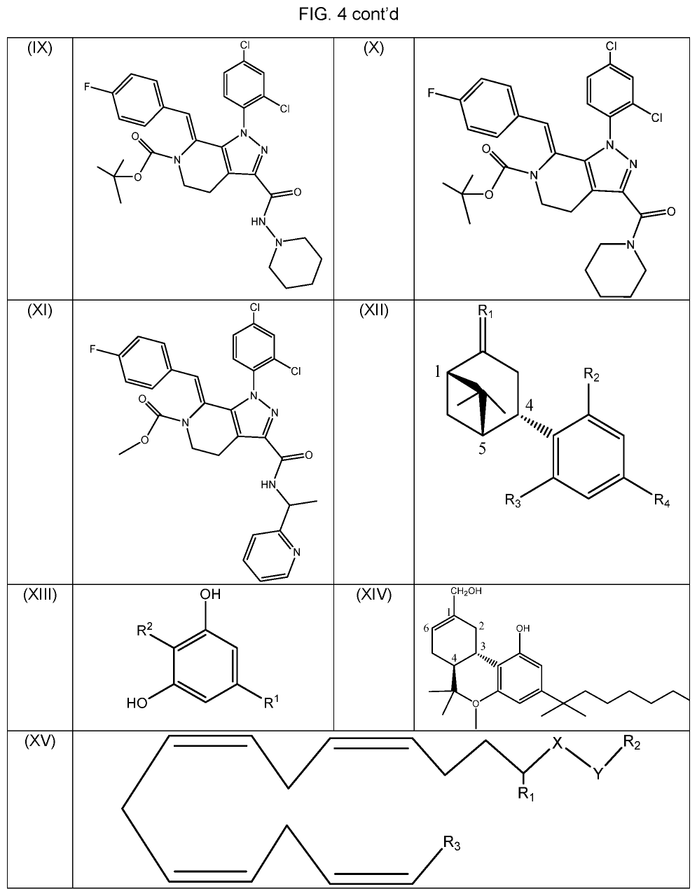

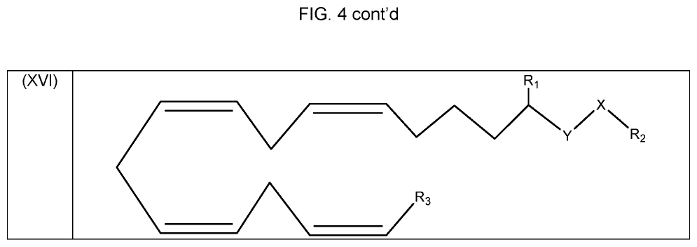

R¹-R³⁶ are independently selected from the group consisting of hydrogen and deuterium. Nabilone derivatives and/or analogs can refer to compounds wherein at least one of R¹-R³⁶ includes deuterium. For the chemical structure of nabilone, see FIG. 4.

Plant-based formulations and synthetic cannabinoid formulations include carriers that lead to rapid onset such as modified amino acids, a surfactant, a detergent, an azone, a pyrrolidone, a glycol, or a bile salt. An amino acid is any carboxylic acid having at least one free amine group and includes naturally occurring, non-naturally occurring and synthetic amino acids. Poly amino acids are either peptides or two or more amino acids linked by a bond formed by other groups which can be linked, e.g. an ester, anhydride, or an anhydride linkage. Peptides are two or more amino acids joined by a peptide bond. Peptides can vary in length from dipeptides with two amino acids to poly peptides with several hundred amino acids. See Chambers Biological Dictionary, editor Peter M. B. Walker, Cambridge, England: Chambers Cambridge, 1989, page 215. Di-peptides, tri-peptides, tetra-peptides, and penta-peptides can also be used.

Carriers which are modified amino acids include acylated fatty acid amino acids (FA-aa) or a salt thereof, which are typically prepared by modifying the amino acid or an ester thereof by acylation or sulfonation. Acylated fatty acid amino acids include N-acylated FA-aa or an amino acid acylated at its alpha amino group with a fatty acid.

Exemplary N-acylated fatty amino acid salts include sodium N-[8-(2-hydroxybenzoyl) amino] caprylate (SNAC). Other names for SNAC include Sodium-N-salicyloyl-8-aminocaprylate, Monosodium 8-(N-salicyloylamino) octanoate, N-(salicyloyl)-8-aminooctanoic acid monosodium salt. monosodium N-{8-(2-hydroxybenzoyl)amino}octanoate, or sodium 8-[(2-hydroxybenzoyl)amino]octanoate. SNAC has the structure:

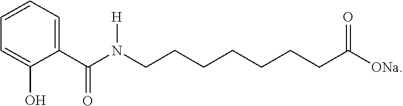

Salts of SNAC may also be used as a carrier.

In particular embodiments, the carriers include:

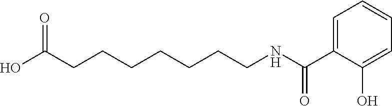 [SMILES: O=C(O)CCCCCCCNC(=O)c1ccccc1O]

In particular embodiments, the carriers include N-[8-(2-hydroxybenzoyl) amino] caprylic acid (NAC).

Other forms of carriers include:

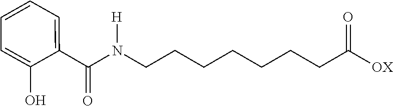

wherein X and Z are independently H, a monovalent cation, a divalent metal cation, or an organic cation. Examples of monovalent cations include sodium and potassium. Examples of divalent cations include calcium and magnesium. Examples of organic cations include ammonium and tetramethylammonium.

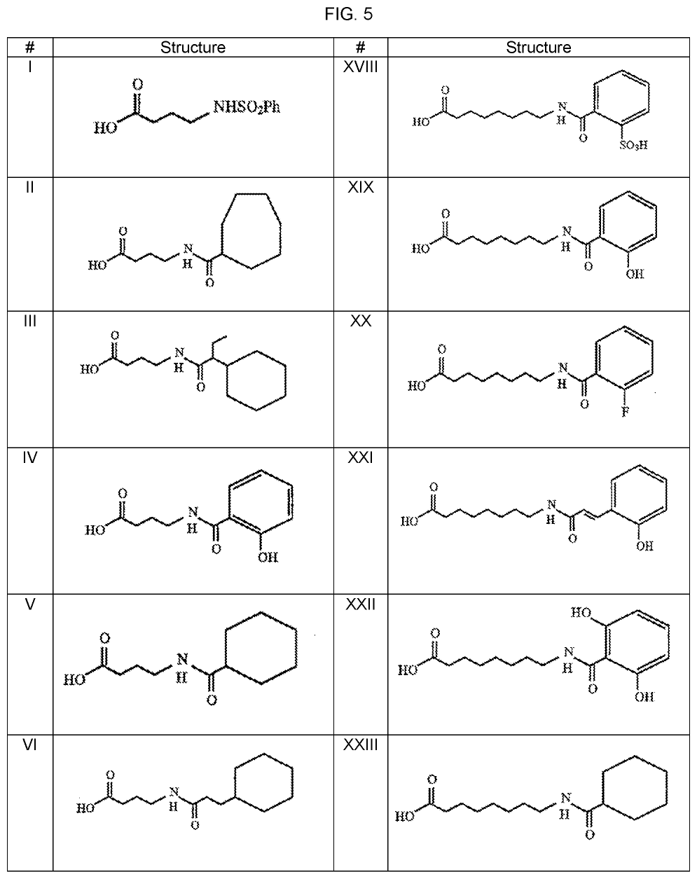

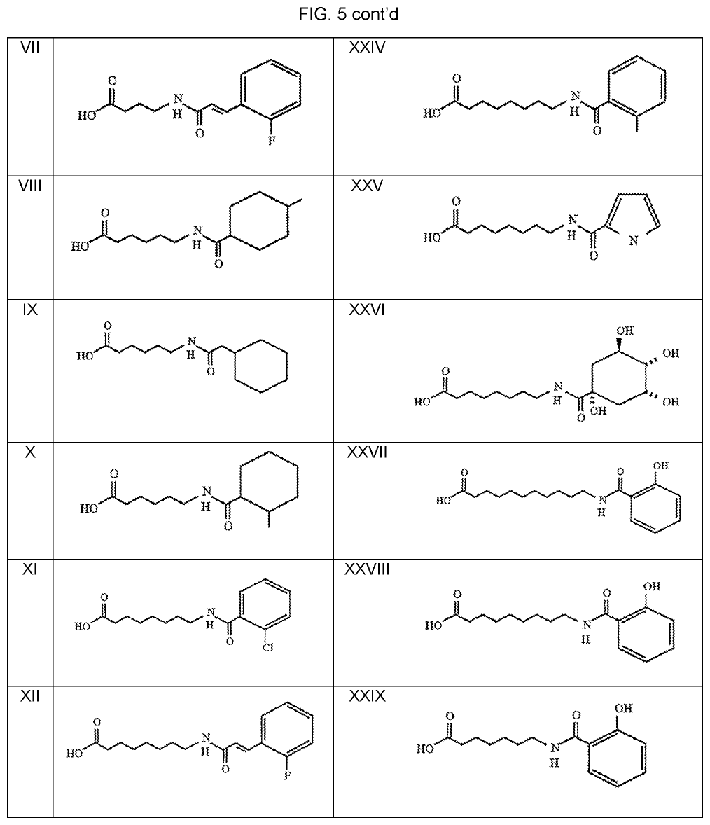

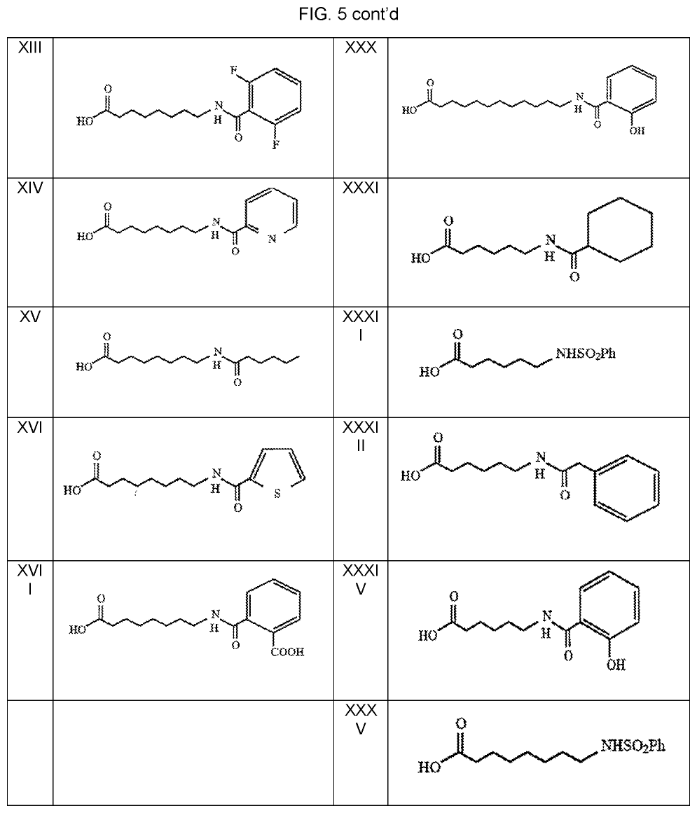

Exemplary modified amino acids, such as N-acylated FA-aas, are provided as compounds I-XXXV (see FIG. 5). Salts of these compounds and other N-acylated FA-aa can also be used as carriers.

Many of the compounds can be readily prepared from amino acids by methods within the skill of those in the art based upon the present disclosure. For example, compounds I-VII are derived from aminobutyric acid. Compounds VIII-X and XXXI-XXIIV are derived from aminocaproic acid. Compounds XI-XXVI and XXXV are derived from aminocaprylic acid. For example, the modified amino acid compounds above may be prepared by reacting the single amino acid with the appropriate modifying agent which reacts with free amino moiety present in the amino acids to form amides. Protecting groups may be used to avoid unwanted side reactions as would be known to those skilled in the art.

The amino acid can be dissolved in aqueous alkaline solution of a metal hydroxide, e.g., sodium or potassium hydroxide, and heated at a temperature ranging between 5° C. and 70° C., in particular embodiments between 10° C. and 40° C., for a period ranging between 1 hour and 4 hours, and in particular embodiments 2.5 hours. The amount of alkali employed per equivalent of NH₂ groups in the amino acid generally ranges between 1.25 and 3 mmole, in particular embodiments between 1.5 and 2.25 mmole per equivalent of NH₂. The pH of the solution generally ranges between 8 and 13, in particular embodiments ranging between 10 and 12.

Thereafter, the appropriate amino acid modifying agent is added to the amino acid solution while stirring. The temperature of the mixture is maintained at a temperature generally ranging between 5° C. and 70° C., in particular embodiments between 10° C. and 40° C., for a period ranging between 1 and 4 hours. The amount of amino acid modifying agent employed in relation to the quantity of amino acid is based on the moles of total free NH₂ in the amino acid. In general, the amino acid modifying agent is employed in an amount ranging between 0.5 and 2.5 mole equivalents, in particular embodiments between 0.75 and 1.25 equivalents, per molar equivalent of total NH₂ group in the amino add.

The reaction is quenched by adjusting the pH of the mixture with a suitable acid, e.g., concentrated hydrochloric acid, until the pH reaches between 2 and 3, The mixture separates on standing at room temperature to form a transparent upper layer and a white or off-white precipitate. The upper layer is discarded, and the modified amino acid is collected from the lower layer by filtration or decantation. The crude modified amino acid is then dissolved in water at a pH ranging between 9 and 13, in particular embodiments between 11 and 13. Insoluble materials are removed by filtration and the filtrate is dried in vacuo. The yield of modified amino acid generally ranges between 30 and 60%, and usually 45%.

If desired, amino acid esters, such as, for example benzyl, methyl, or ethyl esters of amino acid compounds, may be used to prepare the modified amino acids. The amino acid ester, dissolved in a suitable organic solvent such as dimethylformamide, pyridine, or tetrahydrofuran can be reacted with the appropriate amino acid modifying agent at a temperature ranging between 5° C. and 70° C., in particular embodiments 25° C., for a period ranging between 7 and 24 hours. The amount of amino acid modifying agent used relative to the amino acid ester is the same as described above for amino acids. This reaction may be carried out with or without a base such as, for example, triethylamine or diisopropylethylamine.

Thereafter, the reaction solvent is removed under negative pressure and the ester functionality is removed by hydrolyzing the modified amino acid ester with a suitable alkaline solution, e.g. 1N sodium hydroxide, at a temperature ranging between 50° C. and 80° C., in particular embodiments 70° C., for a period of time sufficient to hydrolyze off the ester group and form the modified amino acid having a free carboxyl group. The hydrolysis mixture is then cooled to room temperature and acidified, e.g. aqueous 25% hydrochloric acid solution, to a pH ranging between 2 and 2.5. The modified amino acid precipitates out of solution and is recovered by conventional means such as filtration or decantation. Benzyl esters may be removed by hydrogenation in an organic solvent using a transition metal catalyst.

The modified amino acid may be purified by recrystallization or by fractionation on solid column supports. Suitable recrystallization solvent systems include acetonitrile, methanol and tetrahydrofuran. Fractionation may be performed on a suitable solid column supports such as alumina, using methanolin-propanol mixtures as the mobile phase; reverse phase column supports using trifluoroacetic acid/acetonitrile mixtures as the mobile phase; and ion exchange chromatography using water as the mobile phase. When anion exchange chromatography is performed, in particular embodiments a subsequent 0-500 mM sodium chloride gradient is employed.

In particular embodiments, modified amino acids having the formula

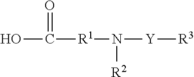

wherein Y is

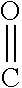

### or SO₂;

R¹ is C₃-C₂₄ alkylene, C₂-C₂₀ alkenylene, C₂-C₂₀ alkynylene, cycloalkylene, or an aromatic, such as arylene;
R² is hydrogen, C₁-C₄ alkyl, or C₂-C₄ alkenyl; and
R³ is C₁-C₇ alkyl, C₃-C₁₀ cycloalkyl, aryl, thienyl, pyrrolo, or pyridyl, and
R³ is optionally substituted by one or more C₁-C₅ alkyl group, C₂-C₄ alkenyl group, F, Cl, OH, OR¹, SO₂, COOH, COOR¹ or, SO₃H;
may be prepared by
reacting in water and the presence of a base a lactam having the formula

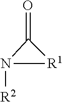

with a compound having the formula R³—Y—X, wherein Y, R¹, R², and R³ are as above and X is a leaving group. A lactam as shown in the above formula can be prepared, for example by the method described in Olah et al., Synthesis, 537-538 (1979).

In particular embodiments, modified amino acids also include an amino acid acylated at its alpha amino group with a fatty acid, which can be represented by the general formula A-X, wherein A is the alpha-amino acid residue and X is a fatty acid attached by acylation to A's alpha-amino group. The amino acids include cationic and non-cationic amino acids. In particular embodiments the term “non-cationic amino acid” refers to an amino acid selected from the group consisting of non-polar hydrophobic amino acids, polar non-charged amino acids, and polar acidic amino acids. In particular embodiments the term “non-cationic amino acid” as used herein refers to amino acids selected from the group consisting of Alanine (Ala), Valine (Val), Leucine (Leu), Isoleucine (Ile), Phenylalanine (Phe), Tryptophan (Trp), Methionine (Met), Proline (Pro), Sarcosine, Glycine (Gly), Serine (Ser), Threonine (Thr), Cysteine (Cys), Tyrosine (Tyr), Asparagine (Asn), and Glutamine (Gin), Aspartic acid (Asp), and Glutamic acid (Glu).

In particular embodiments, the acylated FA-aa includes an alpha amino acid residue of a non-polar hydrophobic amino acid. In particular embodiments, the acylated FA-aa may be represented by the general formula A-X, wherein A is the amino acid residue of a non-polar hydrophobic amino acid and X is a fatty acid attached by acylation to A's alpha-amino group. In particular embodiments the term “non-polar hydrophobic amino acid” as used herein refers to categorisation of amino acids used by the person skilled in the art. In particular embodiments the term “non-polar hydrophobic amino acid” refers to an amino acid selected from the group consisting of Ala, Val, Leu, Ile, Phe, Trp, Met, Pro, and Sarcosine.

In particular embodiments, the acylated FA-aa includes the amino acid residue of a polar non-charged amino acid. In particular embodiments the acylated FA-aa may be represented by the general formula A-X, wherein A is the amino acid residue of a polar non-charged amino acid and X is a fatty acid attached by acylation to A's alpha-amino group. In particular embodiments the term “polar non-charged amino acid” as used herein refers to categorisation of amino acids used by the person skilled in the art. In particular embodiments the term “polar non-charged amino acid” refers to an amino acid selected from the group consisting of Gly, Ser, Thr, Cys, Tyr, Asn, and Gln.

In particular embodiments, the acylated FA-aa includes the amino acid residue of a polar acidic amino acid. In particular embodiments, the acylated FA-aa may be represented by the general formula A-X, wherein A is the amino acid residue of a polar acidic amino acid and X is a fatty acid attached by acylation to A's alpha-amino group. In particular embodiments, the term “polar acidic amino acid” as used herein refers to categorisation of amino acids used by the person skilled in the art. In particular embodiments, the term “polar acidic amino acid” refers to an amino acid selected from the group consisting of Asp and Glu.

In particular embodiments, the amino acid residue of the acylated FA-aa includes the amino acid residue of an amino acid that is not encoded by the genetic code. Modifications of amino acids by acylation may be readily performed using acylation agents known in the art that react with the free alpha-amino group of the amino acid.

In particular embodiments, the alpha-amino acids or the alpha-amino acid residues herein are in the L-form unless otherwise stated.

In particular embodiments, the amino acid residue is in the free acid form and/or a salt thereof, such as a sodium (Na+) salt thereof.

Exemplary embodiments of acylated FA-aas may be represented by the general Fa-aa formula I:

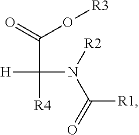

wherein R1 is an alkyl or aryl group including 5 to 19 carbon atoms; R2 is H (i.e. hydrogen), CH₃ (i.e. methyl group), or covalently attached to R4 via a (CH₂)₃ group; R3 is H or absent; and R4 is an amino acid side chain or covalently attached to R2 via a (CH₂)₃ group; or a salt thereof.

The FA-aa can be acylated with a fatty acid including a substituted or unsubstituted alkyl group consisting of 5 to 19 carbon atoms. In particular embodiments, the alkyl group consists of 5 to 17 carbon atoms. In particular embodiments, the alkyl group consists of 5-15 carbon atoms. In particular embodiments the alkyl group consists of 5-13 carbon atoms. In particular embodiments the alkyl group consists of 6 carbon atoms.

In particular embodiments, the acylated FA-aa is soluble at intestinal pH values, particularly in the range pH 5.5 to 8.0, such as in the range pH 6.5 to 7.0. In particular embodiments, the acylated FA-aa is soluble below pH 9.0.

In particular embodiments, the acylated FA-aa has a solubility of at least 5 mg/mL. In particular embodiments, the acylated FA-aa has a solubility of at least 10 mg/mL. In particular embodiments, the acylated FA-aa has a solubility of at least 20 mg/mL. In particular embodiments, the acylated FA-aa has a solubility of at least 30 mg/mL. In particular embodiments, the acylated FA-aa has a solubility of at least 40 mg/mL. In particular embodiments, the acylated FA-aa has a solubility of at least 50 mg/mL. In particular embodiments, the acylated FA-aa has a solubility of at least 60 mg/mL. In particular embodiments, the acylated FA-aa has a solubility of at least 70 mg/mL. In particular embodiments, the acylated FA-aa has a solubility of at least 80 mg/mL. In particular embodiments, the acylated FA-aa has a solubility of at least 90 mg/mL. In particular embodiments, the acylated FA-aa has a solubility of at least 100 mg/mL. In particular embodiments, solubility of the acylated FA-aa is determined in an aqueous solution at a pH value 1 unit above or below pKa of the FA-aa at 37° C. In particular embodiments, solubility of the acylated FA-aa is determined in an aqueous solution at pH 8 at 37° C. In particular embodiments, solubility of the acylated FA-aa is determined in an aqueous solution at a pH value 1 unit above or below pl of the FA-aa at 37° C. In particular embodiments, solubility of the acylated FA-aa is determined in an aqueous solution at a pH value 1 units above or below pl of the FA-aa at 37° C., wherein said FA-aa two or more ionisable groups with opposite charges. In particular embodiments, solubility of the FA-aa is determined in an aqueous 50 mM sodium phosphate buffer, pH 8.0 at 37° C.

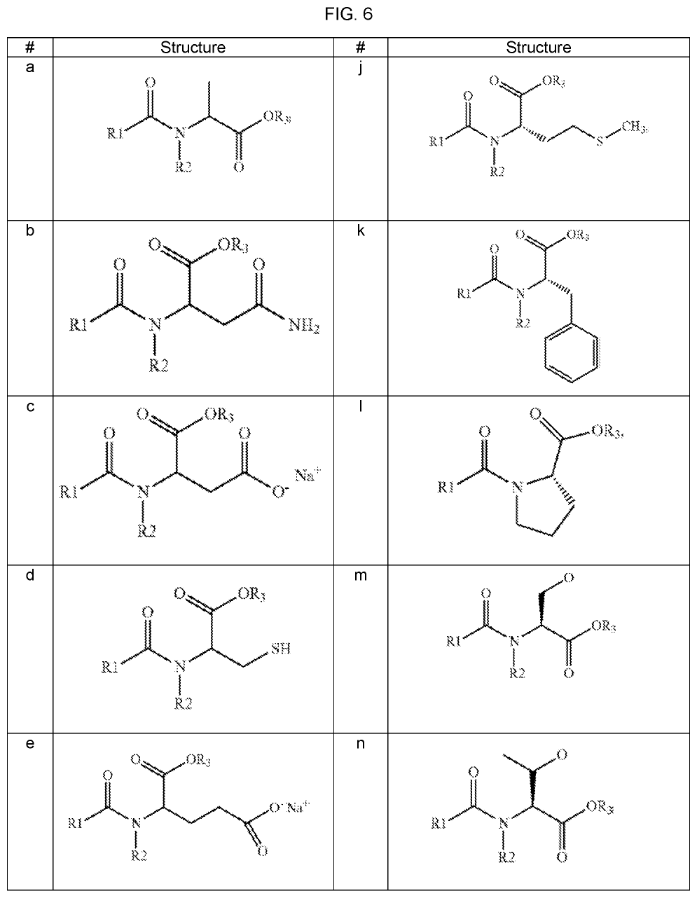

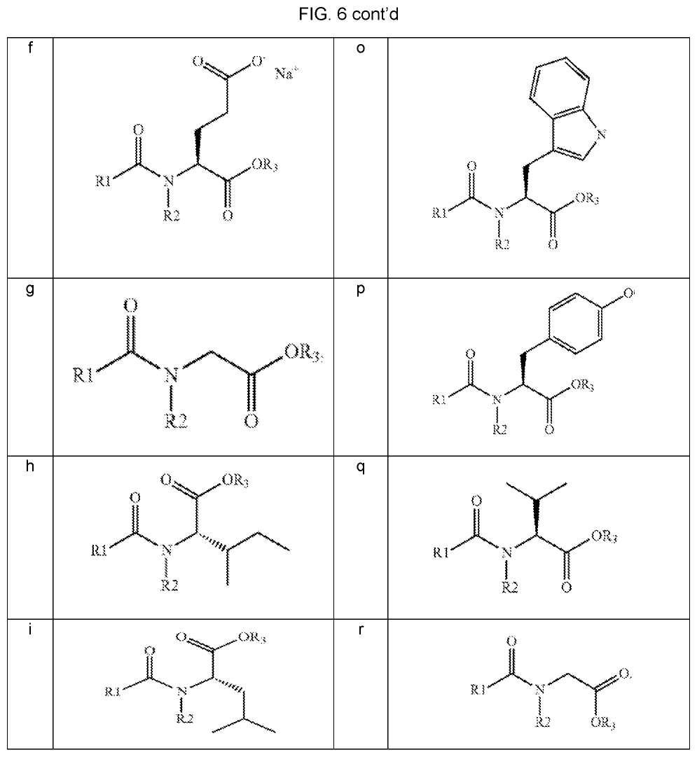

In particular embodiments the acylated FA-aa is selected from the group consisting of formula (a), (b), (c), (d), (e), (f), (g), (h), (i), (j), (k), (I), (m), (n), (o), (p), (q), and (r), wherein R1 is an alkyl group including 5 to 19 carbon atoms, R2 is H (i.e. hydrogen) or CH₃ (i.e. methyl group), and R3 is H; or a salt or the free acid form thereof. Formulas (a), (b), (c), (d), (e), (f), (g), (h), (i), (j), (k), (I), (m), (n), (o), (p), (q), and (r) are provided in FIG. 6.

In particular embodiments, the acylated FA-aa can be selected from one or more of sodium N-dodecanoyl alaninate, N-dodecanoyl-L-alanine, sodium N-dodecanoyl isoleucinate, N-dodecanoyl-L-isoleucine, sodium N-dodecanoyl leucinate, N-dodecanoyl-L-leucine, sodium N-dodecanoyl methioninate, N-dodecanoyl-L-methionine, sodium N-dodecanoyl phenylalaninate, N-dodecanoyl-L-phenylalanine, sodium N-dodecanoyl prolinate, N-dodecanoyl-L-proline, sodium N-dodecanoyl tryptophanate, N-dodecanoyl-L-tryptophane, sodium N-dodecanoyl valinate, N-dodecanoyl-L-valine, sodium N-dodecanoyl sarcosinate, N-dodecanoyl-L-sarcosine, sodium N-oleoyl sarcosinate, sodium N-decyl leucine, sodium N-decanoyl alaninate, N-decanoyl-L-alanine, sodium N-decanoyl leucinate, N-decanoyl-L-leucine, sodium N-decanoyl phenylalaninate, N-decanoyl-L-phenylalanine, sodium N-decanoyl valinate, N-decanoyl-L-valine, sodium N-decanoyl isoleucinate, N-decanoyl-L-isoleucine, sodium N-decanoyl methioninate, N-decanoyl-L-methionine, sodium N-decanoyl prolinate, N-decanoyl-L-proline, sodium N-decanoyl threoninate, N-decanoyl-L-threonine, sodium N-decanoyl tryptophanate, N-decanoyl-L-tryptophane, sodium N-decanoyl sarcosinate, N-decanoyl-L-Sarcosine, N-dodecanoyl asparaginate, N-dodecanoyl-L-asparagine, sodium N-dodecanoyl aspartic acid, N-dodecanoyl-L-aspartic acid, sodium N-dodecanoyl cysteinate, N-dodecanoyl-L-cysteine, sodium N-dodecanoyl glutaminate, N-dodecanoyl-L-glutamine, sodium N-dodecanoyl glycinate, N-dodecanoyl-L-glycine, sodium N-dodecanoyl serinate, N-dodecanoyl-L-serine, sodium N-dodecanoyl threoninate, N-dodecanoyl-L-threonine, sodium N-dodecanoyl tyrosinate, N-dodecanoyl-L-tyrosine, sodium N-decanoyl asparaginate, N-decanoyl-L-asparagine, sodium N-decanoyl aspartic acid, N-decanoyl-L-aspartic acid, sodium N-decanoyl cysteinate, N-decanoyl-L-cysteine, sodium N-decanoyl glutaminate, N-decanoyl-L-glutamine, sodium N-decanoyl glycinate, N-decanoyl-L-glycine, sodium N-decanoyl serinate, N-decanoyl-L-serine, sodium N-decanoyl tyrosinate, N-decanoyl-L-tyrosine, sodium N-dodecanoyl asparaginate, sodium N-dodecanoyl glutamic acid, N-dodecanoyl-L-glutamic acid, sodium N-decanoyl glutamic acid, N-decanoyl-L-glutamic acid, Amisoft HS-11 P (sodium Stearoyl Glutamate, Amisoft MS-11 (sodium Myristoyl Glutamate), Amisoft LS-11 (sodium Dodecanoyl Glutamate), Amisoft CS-11 (sodium Cocoyl Glutamate), sodium N-cocoyl glutamate, Amisoft HS-11 P, Amisoft HS-11 P (sodium N-stearoyl glutamate), (sodium N-myristoyl glutamate)), (sodium N-dodecanoyl glutamate), and Amisoft HS-11 P.

The following acylated FA-aas are commercially available:

| | | Provider |
|---|---|---|
| Brand Name | Chemical Name | (per 14 APR. 2011) |
| Hamposyl L-95 | sodium N-dodecanoyl sarcosinate | Chattem Chemicals |
| Hamposyl O | sodium N-oleoyl sarcosinate | Chattem Chemicals |
| Hamposyl C | sodium N-cocoyl sarcosinate | Chattem Chemicals |
| Hamposyl L-30 | sodium N-dodecanoyl sarcosinate | Chattem Chemicals |
| Amisoft HS-11 P | sodium N-stearoyl glutamate | Ajinomoto |
| Amisoft LS-11 | sodium N-dodecanoyl glutamate | Ajinomoto |
| Amisoft CS-11 | sodium N-cocoyl glutamate | Ajinomoto |
| Amisoft MS-11 | sodium N-myristoyl glutamate | Ajinomoto |
| Amilite GCS-11 | sodium N-cocoyl glycinate | Ajinomoto |

In particular embodiments the terms “fatty acid N-acylated amino acid”, “fatty acid acylated amino acid”, or “acylated amino acid” are used interchangeably herein and refer to an amino acid that is acylated with a fatty acid at its alpha-amino group.

Particular embodiments utilize vegetable matter and/or synthetic cannabinoids with low solubility, or very low solubility. Particular embodiments utilize vegetable matter and/or synthetic cannabinoids that are essentially water insoluble. In particular embodiments, solubility in water is defined as low to zero by the United States pharmacopeia (USP 32) according to the amount of water necessary for the dissolution of one part of solute: Low solubility: 100 to 1000 parts of water necessary for dissolution of one part of solute; very low solubility: 1000 to 10 000 parts of water necessary; essentially water insoluble more than 10 000 parts of water necessary. Ata basic pH, however, SNAC and other modified amino acids and FA-aas described herein are water soluble. Thus, the administration benefits, as described herein could not be reasonably predicted. In particular embodiments, very low solubility can refer to a solubility in water or an aqueous solution of less than 1 mg/ml, less than 0.1 mg/ml, or less than 0.01 mg/ml.

In particular embodiments, N-acylated fatty amino acids act as rapid onset absorption enhancing agents, thereby creating an administration benefit. Absorption enhancing agents refer to compounds that promote gastrointestinal absorption. Absorption enhancing agents can improve drug absorption by improving the solubility of the drug in the gastrointestinal tract or by enhancing membrane penetration, as compared to a formulation that does not include the absorption enhancing agents. Additional examples of absorption enhancing agents include surfactants, detergents, azones, pyrrolidones, glycols or bile salts.

In particular embodiments, N-acylated fatty amino acids act as rapid onset bioavailability enhancing agents. Bioavailability refers to the fraction of active ingredient that is actually absorbed by a subject and reaches the bloodstream. In particular embodiments, bioavailability enhancing agents increase the fraction of active ingredient in the bloodstream or result in detection of active ingredient in the bloodstream earlier in time, as compared to a formulation that does not include the bioavailability enhancing agent.

In particular embodiments, additional administration benefits created by rapid onset absorption enhancing agents and/or bioavailability enhancing agents include faster onset of action, higher peak concentrations, faster time to peak concentrations, as compared to a control plant-based formulation or oral formulation based on the same, similar in all aspects but for inclusion of the absorption enhancing agents and/or bioavailability enhancing agents.

Embodiments utilizing rapid onset absorption enhancing agents and/or bioavailability enhancing agents (e.g., and in particular embodiments, N-acylated fatty amino acids) can be beneficial because many oral plant-based formulations and synthetic cannabinoid formulations designed to address various physiological conditions are inadequate because they are characterized by a delayed onset of action, and low bioavailability. Delayed onset of action presents challenges in clinical indications that require rapid therapeutic effect (e.g. pain and migraine); and low bioavailability requires patients to ingest significantly higher doses than would be required by alternative dosing forms (e.g. smoking, vaping). Particular embodiments disclosed herein provide plant-based formulation and synthetic cannabinoid oral formulations with improved bioavailability and shorter time to onset of therapeutic effect.

As indicated, particular embodiments disclosed herein also provide extended action by including at least one sustained release system. Sustained release systems can create administration benefits by maintaining the fraction of active ingredient in the bloodstream for a longer period of time than a comparable formulation without the sustained release system and/or maintaining a therapeutic or physiological effect for a longer period of time than a comparable formulation without the sustained release system.

The description below regarding materials and methods to formulate tablets and particles can interchangeably be applied whether making a tablet or a particle. That is, unless the context clearly dictates otherwise, the description of materials and methods to make tablets can be applied to the making of particles, and the description of materials and methods to make particles can be applied to the making of tablets.

Release-Controlling Matrices. In particular embodiments, extended action materials are materials that form a matrix and permit release of vegetable matter and/or synthetic cannabinoids at a desired rate in an aqueous medium. The release-controlling matrix material can be chosen to achieve a desired in vitro and/or in vivo release rate. In these embodiments, the vegetable matter and/or synthetic cannabinoids are embedded within the matrix.

Release-controlling matrix materials can be hydrophilic and/or hydrophobic polymers. Release-controlling matrix materials include, for example acrylic polymers, waxes, alkylcelluloses, shellac, zein, hydrogenated vegetable oil, hydrogenated castor oil, and combinations including one or more of the foregoing materials.

Suitable acrylic polymers include, for example, acrylic acid and methacrylic acid copolymers, aminoalkyl methacrylate copolymer, cyanoethyl methacrylate, ethoxyethyl methacrylates, glycidyl methacrylate copolymers, methacrylic acid alkylamide copolymer, methyl methacrylate copolymers, poly(acrylic acid), poly(methacrylic acid), poly(methacrylic acid anhydride), poly(methyl methacrylate), poly(methyl methacrylate) copolymer, polyacrylamide, polymethacrylate, methyl methacrylate, and combinations including one or more of the foregoing polymers.

Suitable modified celluloses include, for example, alkyl celluloses and hydroxyalkyl celluloses. Alkyl cellulose includes, for example, methyl cellulose and ethyl cellulose. Hydroxyalkyl cellulose includes, for example, hydroxyethyl cellulose, hydroxypropyl cellulose, hydroxypropylmethyl cellulose (HPMC), hydroxypropylethyl cellulose, hydroxypropylpropyl cellulose, and hydroxypropylbutyl cellulose. Those of ordinary skill in the art will appreciate that other cellulosic polymers, including other alkyl or hydroxyalkyl cellulosic polymers, can be substituted for part or all of the ethyl cellulose and/or hydroxyalkyl cellulose.

Other suitable hydrophobic materials are water-insoluble with more or less pronounced hydrophobic trends. In particular embodiments, the hydrophobic material can have a melting point of 30° C. to 200° C., in particular embodiments 45° C. to 90° C. The hydrophobic material can include neutral and/or synthetic waxes, fatty alcohols (such as lauryl, myristyl, stearyl, cetyl or cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and combinations including one or more of the foregoing materials. Suitable waxes include beeswax, glycowax, castor wax, carnauba wax and waxlike substances, e.g., material normally solid at room temperature and having a melting point of from 30° C. to 100° C., and combinations including one or more of the foregoing waxes.

In particular embodiments, the release-controlling matrix materials can include digestible, long chain (e.g., C₈-C₅₀, or C₁₂-C₄₀), substituted or unsubstituted hydrocarbons, such as fatty acids, fatty alcohols, glyceryl esters of fatty acids, mineral and vegetable oils, waxes, and combinations including one or more of the foregoing materials. In particular embodiments, hydrocarbons having a melting point of between 25° C. and 90° C. can be used. Of these long chain hydrocarbon materials, in particular embodiments, fatty (aliphatic) alcohols can be used. The oral formulation can include up to 60% by weight of at least one digestible, long chain hydrocarbon. In particular embodiments, the oral formulation can include up to 60% by weight of at least one polyalkylene glycol.

In particular embodiments, the release-controlling matrix materials can include polylactic acid, polyglycolic acid or a co-polymer of lactic and glycolic acid.

In particular embodiments, release-controlling matrix materials include a high-viscosity HPMC and a low-viscosity HPMC. In particular embodiments, the high-viscosity HPMC is in the range of from 80,000 cps to 120,000 cps, and in particular embodiments 100,000 cps. In particular embodiments, the low-viscosity HPMC can be in the range of from 2,000 cps to 20,000 cps, and in particular embodiments, 4,000 cps. In particular embodiments, the weight ratio between the high-viscosity HPMC and the low-viscosity HPMC can be from 10:1 to 1:1. In particular embodiments, the weight ratio between the high-viscosity HPMC and the low-viscosity HPMC can be from 2.6:1 to 1:1.

In particular embodiments, a release-controlling matrix material can be prepared by mixing a high-viscosity HPMC having a viscosity of from 80,000 cps to 120,000 cps and a low-viscosity HPMC having a viscosity of 2,000 cps to 20,000 cps at a weight ratio in the range of from 10:1 to 1:1.

Ion exchange resins with various degrees of crosslinking and a range of binding capacities can also be used to create release-controlling matrices. In particular embodiments, the oral formulations can be prepared by contacting an ion exchange resin with vegetable matter and/or synthetic cannabinoids to form a vegetable matter and/or synthetic cannabinoids/ion exchange resin core or complex.

Typically suitable ion exchange resins are in the form of ion exchange resin particles. Stirring the ion-exchange resin particles in a solution of the selected vegetable matter and/or synthetic cannabinoids is usually sufficient to achieve binding of vegetable matter and/or synthetic cannabinoids onto the resin particles.

In particular embodiments, loading of the resin is suitably carried out at a pH that facilitates binding of vegetable matter and/or synthetic cannabinoids. Some ion exchange resins can require “activation” by rinsing with a solution of acid or base, prior to loading with vegetable matter and/or synthetic cannabinoids. Such activation requirements are known to those skilled in the art of working with ion exchange resin materials. Specific requirements for individual ion exchange resin materials can be obtained from the resin manufactures. In particular embodiments, formed particles can be spherical to enable substantially complete coating of the particles.

Enteric Coatings. In particular embodiments, oral formulations can include an enteric coating. An enteric coating refers to a coating material which remains substantially intact in the acid environment of the stomach, but which dissolves in the neutral environment of the intestines. The stomach has a pH between 1 and 3, duodenum between 5 and 7, ascending colon between 7 and 8, and the jejunum has a pH of 6.5.

In particular embodiments, an enteric coating is a coating that prevents release of the vegetable matter and/or synthetic cannabinoids until the dosage form reaches the small intestine. Examples of suitable enteric coating materials include acrylic acid polymers; cellulose acetate butyrate; cellulose acetate hexahydrophthalate; cellulose acetate maleate; cellulose acetate phthalate (CAP); cellulose acetate propionate; cellulose acetate succinate; cellulose acetate trimellitate; cellulose polymers; cellulose propionate phthalate; ethyl methacrylate-ethylmethacrylate-chlorotrimethylammonium ethyl methacrylate copolymer; hydroxypropyl cellulose; hydroxypropyl methylcellulose acetate succinate (HPMCAS); HPMC, hydroxypropyl methylcellulose hexahydrophthalate; hydroxypropyl methylcellulose phthalate (HPMCP); methacrylic acid/methacrylate polymer; methacrylic acid/methyl methacrylate copolymer; methacrylic resins commercially available under the trade name EUDRAGIT® (Evonik ROhm GmbH, Germany); polyvinyl acetate phthalate (PVAP); and blends and copolymers thereof. Other examples include natural resins, such as shellac, SANDARAC, and copal collophorium. Enteric coated oral formulations can be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et. al., (Media, Pa.: Williams and Wilkins, 1995).

Barrier Layers. In particular embodiments, oral formulations can include a barrier layer. As used herein, a barrier layer slows the release of vegetable matter and/or synthetic cannabinoids from an oral formulation but is chosen independently of pH conditions of the gastric tract.

In particular embodiments, barrier layers can include one or more swellable, erodible and/or gellable polymers.

Examples of gellable polymers include guar gums; high-molecular weight polysaccharides including mannose and galactose sugars, or guar derivatives such as hydropropyl guar (HPG), carboxymethyl guar (CMG), and carboxymethylhydroxypropyl guar (CMHPG); cellulose derivatives such as hydroxyethylcellulose (HEC) or hydroxypropylcellulose (HPC) and carboxymethyl hydroxyethylcellulose (CMHEC); xanthan; diutan; scleroglucan; polyacrylamide; polyvinyl alcohol; polyethylene glycol; polypropylene glycol; and polyacrylate polymers.

Examples of swellable polymers swellable polymers include crosslinked poly(acrylic acid); a poly(alkylene oxide) (e.g., polymers which contain as a unit, ethylene oxide, propylene oxide, ethylene oxide, or propylene oxide); a poly(vinyl alcohol); a poly(vinyl pyrrolidone); a polyurethane hydrogel, a maleic anhydride polymer, such as a maleic anhydride copolymer; a cellulose polymer (e.g., cellulose, hydroxymethylcellulose, hydroxyethyl cellulose, hydroxypropyl cellulose, hydroxypropylmethyl cellulose (also known as hypromellose), and carboxymethyl cellulose); a polysaccharide (e.g., dextran, xanthan gum, gellan gum, welan gum, rhamsan gum, sodium alginate, calcium alginate, chitosan, gelatin, and maltodextrin); starch; starch based polymers (e.g., hydrolyzed starch polyacrylonitrile graft copolymers, starch-acrylate-acrylamide copolymers).

Commercially available swellable polymers include PolyOX 303® (Poly(ethylene oxide), molecular weight 7,000,000); PolyOX WSR N-12K (Poly(ethylene oxide), molecular weight 1,000,000), PolyOX WSR N-60K (Poly(ethylene oxide), molecular weight 2,000,000), PolyOX WSR 301 (Poly(ethylene oxide), molecular weight 4,000,000), PolyOX WSR Coagulant, PolyOX WSR 303, PolyOX WSR 308, NFgrade™ (Poly(ethylene oxide) molecular weight 1,000,000); PolyOX WSR N80™ (Poly(ethylene oxide), molecular weight 200,000); METHOCELO F4M (hydroxypropyl methylcellulose); METHOCEL A15C (methylcellulose), METHOCEL A18M, METHOCEL K4M (hydroxypropyl methylcellulose 2208), METHOCEL K100 (hydroxypropyl methylcellulose 2910) METHOCEL E10M (hydroxypropyl methylcellulose 2910), METHOCEL E4M (hydroxypropyl methylcellulose); METHOCEL K15MP (hydroxypropyl methylcellulose); each available from Dow Chemical Company, Midland Mich.

Other examples of commercially available swellable polymers include BLANOSEO cellulose gum, including Blanose cellulose gum, grad 7H4 (sodium carboxymethyl cellulose), ECN7 Pharmaceutical Grade™ (Ethyl cellulose); and ECN22 Pharmaceutical Grade™ (Ethylcellulose); Klucel HF™ (hydroxypropyl cellulose, molecular weight 1,150,000); Klucel NF™ (hydroxypropyl cellulose); Klucel MF (hydroxypropyl cellulose, molecular weight 850,000), Klucel GF (hydroxypropyl cellulose, molecular weight 370,000), Klucel JF (hydroxypropyl cellulose, molecular weight 140,000), Klucel LF (hydroxypropyl cellulose, molecular weight 95,000), Klucel EF (hydroxypropyl cellulose, molecular weight 80,000), and Natrosol 250HX (hydroxyethylcellulose) each available from Hercules Incorporated, Wilmington, Del. (supplied by Aqualon).

Other examples of commercially available swellable polymers include L-HPC Grade 11™ (Low Substituted hydroxypropyl cellulose), available from Shin-Etsu Chemical Co., Ltd., via Biddle Sawyer Corp., New York, N.Y.).

Other examples of commercially available swellable polymers include Primellose™ (Croscarmellose Sodium); Monkey 4™ (Sodium Starch Glycolate); each (available from Avebe, via Generichem Corporation, Totowa, N.J.).

Other examples of commercially available swellable polymers include Carbopol 974P™ (polyacrylic acid cross-linked with polyalkenyl ethers or divinyl glycol); Carbopol 934P (polyacrylic acid); Carbopol 971P (polyacrylic acid cross-linked with polyalkenyl ethers or divinyl glycol); each available from Noveon, Inc., Cleveland, Ohio.

Other examples of commercially available swellable polymers include polyvinyl alcohols available from DuPont, such as Elvanol® 71-30, Elvanol® 85-30, Elvanol® 50-42, and Elvanol®. HV.

Examples of erodible polymers include polyethylene oxide, in particular polyethylene oxide water soluble resins (Polyox® WSR Coagulant and Polyox®. WSR-303); glyceryl fatty acid esters, e.g., glyceryl behenate, glyceryl monostearate, glycerol distearate, glycerol monooleate, acetylated monoglycerides, tristearin, tripalmitin, cetyl esters wax, glyceryl palmitostearate, and glyceryl behenate; hydrogenated castor oil; cellulose derivatives, e.g., hydroxyethyl cellulose, hydroxypropyl cellulose, hydroxypropyl methylcellulose, ethylhydroxy ethylcellulose, methylethyl cellulose, carboxymethyl cellulose, and carboxymethyl ethylcellulose; pullulan; polyvinyl pyrrolidone; polyvinyl alcohol; and polyvinyl acetate. In particular embodiments, the water swellable or water soluble or erodible polymers include polyethylene oxide water soluble resins, glyceryl behenate, hydroxyethyl cellulose, hydroxypropyl methylcellulose, and polyvinyl pyrrolidone.

In particular embodiments, the swellable, erodible, and/or gellable polymer is HPMC. In particular embodiments, the average molecular weight of the HPMC is from 1000 to 4,000,000, or from 2000 to 2,000,000. In particular embodiments, the barrier layer(s) include METHOCEL® (Dow Chemical Company, Midland, Mich.) E5, an HPMC having a methoxy content of 28-30%, a hydroxypropoxyl content of 7-12%, and an apparent viscosity, as measured by rotation, of 4.2-6.1 mPa (Colorcon, West Point, Pa.). In particular embodiments, the barrier layer(s) include METHOCEL® E50, an HPMC having a methoxy content of 28-30%, a hydroxypropoxyl content of 7-12%, and an apparent viscosity, as measured by rotation, of 39-59 mPa (Colorcon, West Point, Pa.). In particular embodiments, one barrier layer includes METHOCEL® E5 and a second barrier layer includes METHOCEL® E50.

Additional examples of barrier layer materials that can be used include: cellulose acetate, CAP, cellulose acetate trimellitate, hydroxy propyl methyl cellulose acetate succinate, HPMCP, PVAP, carboxy methyl ethyl cellulose, shellac, ethyl cellulose, polyvinyl acetate, EUDRAGIT®, such as EUDRAGIT® L 12.5 (solution), EUDRAGIT® L 100 (powder), EUDRAGIT® S 12.5 (solution), EUDRAGIT® S 100 (powder): anionic polymers based on methacrylic acid and methacrylates with —COOH functional groups, EUDRAGIT® L 100-55 (powder) and EUDRAGIT® L30 D-55 (solution): anionic polymers of methacrylic acid and ethacrylates with —COOH functional groups, EUDRAGIT® RL (RL 12.5, RLIOO, RL PO, RL 30 D) and EUDRAGIT® RS (RS 12.5, RS 100, RS PO, RL 30 D): Copolymers of acrylate and methacrylates with quarternary ammonium groups as functional group. In particular embodiments, shellac, optionally including talc to assist slow permeation of water through the shellac can be used as a barrier layer. Typically such barrier layers can include shellac including 30-50 wt % of talc, in particular embodiments, 40±5 wt %.

In particular embodiments, a coating operation is continued until the particles have one or more barrier layers suitable to provide a desired extended action profile, for example as determined using the European Pharmacopoeia dissolution test, for example so that after 1 hour 15-30%, after 4 hours 45-70%, after 8 hours more than 70%, and after 12 hours more than 90% of the vegetable matter and/or synthetic cannabinoid content is released into solution.

In particular embodiments, a coating or layer surrounding a matrix includes 0.25 to 40% by weight of the oral formulation. In particular embodiments, an extended action coating surrounding an extended action matrix core includes 0.5 to 20% and in particular embodiments 1 to 10% by weight of the oral formulation.

In particular embodiments, the coating or layer surrounding a matrix can include, by weight, 40 to 95% polymer (e.g. EUDRAGIT® L30D-55) and 5 to 60% plasticizer (e.g. triethyl citrate, polyethylene glycol). The relative proportions of ingredients, notably the ratio of methacrylic polymer to plasticizer can be varied according to methods known to those of skill in the art of pharmaceutical formulation.

In particular embodiments, the weight ratio of a coating or layer surrounding a matrix can be 1:30 to 3:10. In particular embodiments, the weight ratio of a coating or layer surrounding a matrix can be 1:10.

In particular embodiments, the weight ratio of a second coating or layer surrounding a matrix can be 1:30 to 3:10. In particular embodiments, the weight ratio of the second coating or layer surrounding a matrix can be 1:10.

In particular embodiments, oral formulations can also include one or more release modulators, which in combination with a release-controlling matrix, enteric coating(s), and/or barrier layer(s) allow for further modulation of the release of vegetable matter and/or synthetic cannabinoids based on the desired release profile of vegetable matter and/or synthetic cannabinoids. Suitable modulators include glyceryl monostearate, triglyceride derivatives, semi-synthetic glycerides, hydrogenated castor oil, glyceryl palmitostearate, cetyl alcohol, polyvinylpyrrolidone, glycerol, ethylcellulose, methylcellulose, sodium carboxymethylcellulose, other natural and/or synthetic substances well known to those of ordinary skill in the art, and combinations thereof. Other suitable modulator(s) also include magnesium stearate, stearic acid, talc, sodium benzoate, boric acid, polyoxyethylenglycols and colloidal silica. In particular embodiments, the concentration of the modulator(s) is from 1% to 25% by weight of the oral formulations. In particular embodiments, the concentration of the modulator(s) is from 5% to 15% by weight of the oral formulations.

In particular embodiments, a first coating or layer includes from 20 to 85% water-insoluble, polymer (e.g. ethylcellulose), 10 to 75% water-soluble polymer (e.g. polyvinylpyrrolidone), and 5 to 30% plasticizer. The relative proportions of ingredients, notably the ratio of water-insoluble, film-forming polymer to water-soluble polymer, can be varied depending on the release profile to be obtained (where a more delayed-release is generally obtained with a higher amount of water-insoluble, film-forming polymer).

In particular embodiments an oral formulation includes a release-controlling matrix core including vegetable matter and/or synthetic cannabinoids, polyvinylalcohol and glyceryl behenate; a first coating or layer of ethylcellulose, polyvinylpyrrolidone and polyethylene glycol, and a second coating or layer of methacrylic acid copolymer type C, triethyl citrate, polyethylene glycol and optionally including silicon dioxide.

In particular embodiments, an oral formulation includes a first coating that includes a water-insoluble, film-forming polymer, together with a plasticizer and a water-soluble polymer. The water-insoluble, film-forming polymer can be a cellulose ether, such as ethylcellulose, a cellulose ester, such as cellulose acetate, polyvinylalcohol, etc. A suitable film-forming polymer is ethylcellulose (available from Dow Chemical under the trade name ETHOCEL®). Other excipients can optionally also be present in the first coating, as for example acrylic acid derivatives (such and EUDRAGIT®), pigments, etc.

In particular embodiments, the oral formulation can include between 1% and 95% (by weight) of the extended action material.

In particular embodiments, an extended action core is coated by compression over the whole surface (or substantially the whole surface) with a rapid onset fast-disintegrating layer, in particular embodiments formulated as a compressed tablet layer or shell.

In particular embodiments, a rapid onset shell substantially surrounds the chosen sustained release system. Rapid onset shells can be suitably applied to the chosen sustained release system in the form of an aqueous solution or a dispersion of a film-forming material. Exemplary film-forming materials include hydroxypropylcellulose, HPMC, gum arabic, polyvinyl pyrrolidone, polyvinyl pyrrolidone-vinylacetate copolymer and copolymerisate of dimethyl amino methacrylic acid/neutral methacrylic acid esthers. In particular embodiments, a rapid onset shell including a mixture of hydroxypropylcellulose and polyvinylpyrrolidone can be used.

Release-controlling matrices, coatings, layers, and rapid onset shells can be prepared into tablet and particle form according to techniques known in the art. For example, vegetable matter and/or synthetic cannabinoids and extended action materials can be prepared by wet granulation techniques and melt extrusion techniques as described in, for example, U.S. Pat. No. 3,939,259; WO01/19901; WO01/72286; and U.S. Pat. No. 4,902,513.

Pharmaceutically acceptable solvents typically used for application of coatings, layers, or shells include water, methanol, ethanol, methylene chloride, and combinations including one or more of the foregoing solvents.

In particular embodiments, methods of applying a coating, layer, or shell to a matrix include spraying or pouring a solution of the coating, layer, or shell material onto the matrix, Other methods of applying coatings, layers, or shells are known, for example as disclosed in U.S. Pat. No. 3,939,259 e.g. by placing uncoated matrix forms including the vegetable matter and/or synthetic cannabinoid in a revolving pan and contacting the matrix with sufficient coating, layer, or shell solution to cover the matrix, followed by drying.

Spansule® (Smith Kline & French Laboratories Corporation, Philadelphia, Pa.) utilizes sugar-based substrates in which vegetable matter and/or synthetic cannabinoids can be incorporated directly or can be dusted or otherwise distributed over the substrate surface, e.g. building up layers including the vegetable matter and/or synthetic cannabinoids. Typically such a substrate can include a substantially spherical particle typically 35-40 mesh (425-500 microns) size including a compacted mixture of sugar and maize starch. Suitable specifications are described in, for example, the US Pharmacopoeia and the National Formulary.

Powder layering processes can also be used. Typically in such a process substrates can be initially sprayed with a mixture of shellac and an acid in a suitable solvent (e.g., ethanol). This applied mixture is then allowed to dry e.g. in an air stream, leaving a tacky coating on the substrate. These coated substrates can then be dusted with powdered vegetable matter and/or synthetic cannabinoids to thereby deposit a layer of the vegetable matter and/or synthetic cannabinoids. The vegetable matter-coated substrates can then be sprayed with another coating of a mixture of shellac, tartaric add and solvent, and this coating is then allowed to dry as before, then another layer of the powdered vegetable matter and/or synthetic cannabinoids are dusted onto the substrates e.g. in a rotating pan. This process is repeated until the desired amount of the vegetable matter and/or synthetic cannabinoids have been deposited onto the substrates.

Matrices coated with vegetable matter and/or synthetic cannabinoids can be prepared, for example, by dissolving or dispersing the vegetable matter and/or synthetic cannabinoids in a solvent and then spraying the solution onto a substrate, for example, sugar spheres NF-21, 18/20 mesh, using a Wurster insert. Optionally, additional ingredients are also added prior to coating the matrices in order to assist vegetable matter and/or synthetic cannabinoid binding to the matrix. The resulting vegetable matter/synthetic cannabinoid matrix can optionally be overcoated to separate the vegetable matter and/or synthetic cannabinoid from the next coat of material, e.g., enteric coating(s), barrier layer(s) and/or shell(s).

In particular embodiments, the oral formulations can be prepared in a small size with a total weight of 200 mg or less. In particular embodiments the oral formulations can be prepared in the range of from 150 mg to 160 mg, and thus they can be easily swallowed, thereby providing convenience for internal use medicine, and compliance and being cost-effective.

As indicated, particular embodiments include a sustained release system within a rapid onset liquid. The liquid phase can include vegetable matter and/or synthetic cannabinoids in a rapid onset form, e.g. in solution or suspension such that the vegetable matter and/or synthetic cannabinoids can begin to be absorbed by the body substantially immediately after oral administration, and the sustained release system can include vegetable matter and/or synthetic cannabinoids in an extended action form. The liquid and solid phases can both include the same vegetable matter and/or synthetic cannabinoids, or the liquid and solid phases can include different vegetable matter and/or synthetic cannabinoids.

WO03/084518 describes a liquid dosage form which includes coated delayed release particles including an active agent dispersed in a liquid phase which also includes an active agent in solution, and from which an active agent is released rapidly upon oral administration.

In particular embodiments, extended action particles within a rapid onset liquid can include beads, ion-exchange resin beads, spheroids, microspheres, seeds, pellets, granules, and other multiparticulate systems (collectively referred to as particles) in order to obtain sustained-release of the vegetable matter and/or synthetic cannabinoid. The multiparticulate systems can also be included as capsules or other suitable unit dosage forms. In particular embodiments, particles, typically spherical, can be 0.5-1.5 mm, e.g. ca. 1.0±0.1 mm diameter, or other shapes of equivalent volume. In particular embodiments, such particles can include 30-99 wt % of vegetable matter and/or synthetic cannabinoid, for example a cannabinoid in the form of THC and/or CBD.

In particular embodiments utilizing a particle-containing liquid, the viscosity of the liquid can be increased to reduce the rate of particle settling. In particular embodiments, the viscosity of the liquid allows particles to be substantially uniformly suspended in the liquid for sufficient time to be ingested after mixing.

In particular embodiments, a suitable viscosity range can be determined using a Brookfield Rheometer, Spindle 2 at a temperature of 21° C. at a speed of 20 rpm. In particular embodiments, a suitable viscosity is 250-1100 mPa*s. In particular embodiments, a suitable viscosity is 400-500 mPa*s. In particular embodiments, the lower limit of a suitable viscosity range results in 50% of particles remaining unsedimented 2 minutes after agitation. In particular embodiments, the lower limit of a suitable viscosity range results in 50% of particles remaining unsedimented 6 minutes after agitation.

Particular viscosities can be achieved by incorporating one or more suitable thickening agents into an aqueous liquid phase. Suitable thickening agents are known in the oral formulation field, and typically include: xantham gum (such as those sold under the trade marks RHODIGEL® (Rhone-Poulenc Industries, Paris, France) or KELTROL® (Kelco Co., San Diego, Calif.) e.g. RHODIGEL® 80 or KELTROL®), gellan gum (E 418), agar (E 406), carrageen (E 407), galactomanan and modified glalactomannanas (locust bean gum (E 410), guar gum (E 412), tara gum (E 407), konjac gum, gelatin, arabic gum (E 414), karaya gum (E 416), starch and starch derivatives (such as E 1404, E 1410, E 1412-E 1414, E 1420, E 1422, E 1440, E 1442, E 1450, E 1451), tamarind, traganth (E413), xanthan gum (E415), pectin (E440) and amided pectin (E440U), cellulose and cellulose derivatives (such as cellulose (E460), microcrystalline cellulose, sodium carboxymethyl cellulose (E466), carboxymethyl cellulose (E466), methyl cellulose (E461), hydroxypropyl cellulose (E463), Methyl ethyl cellulose (E465), hydroxy propyl methyl cellulose (E464), hydroxyethylcellulose), alginates (such as alginic add (E400), ammonium alginate (E403), calcium alginate (E404), potassium alginate (E402), sodium alginate (E401), propylene glycol alginate (E405), polyvinyl alcohol, ghatii gum, silicates (such as Bentonit (aluminium silicate) or Veegum (magnesium aluminium silicate)), arrowroot, sago, treated Eucheuma alga (E407a), dextran, polyvinyl pyrrolidone (E 120 Ia), polyethylene glycol, acrylic add polymers (such as polymethacrylic acid, polymethacrylates), and colloidal silicon dioxide (e.g. Aerosil).

In particular embodiments, the liquid can be an aqueous liquid phase and can include an amount of 40-90 wt %, 40-60 wt %, or 45-55 wt % water or ethanol.

In particular embodiments, the oral formulations include vegetable matter (e.g., plant parts or extracts) and/or synthetic cannabinoids of at least 0.1% w/v or w/w of the oral formulation; at least 1% w/v or w/w of oral formulation; at least 10% w/v or w/w of oral formulation; at least 20% w/v or w/w of oral formulation; at least 30% w/v or w/w of oral formulation; at least 40% w/v or w/w of oral formulation; at least 50% w/v or w/w of oral formulation; at least 60% w/v or w/w of oral formulation; at least 70% w/v or w/w of oral formulation; at least 80% w/v or w/w of oral formulation; at least 90% w/v or w/w of oral formulation; at least 95% w/v or w/w of oral formulation; or at least 99% w/v or w/w of oral formulation.

In particular embodiments, the oral formulations include carrier of at least 0.1% w/v or w/w of the oral formulation; at least 1% w/v or w/w of oral formulation; at least 10% w/v or w/w of oral formulation; at least 20% w/v or w/w of oral formulation; at least 30% w/v or w/w of oral formulation; at least 40% w/v or w/w of oral formulation; at least 50% w/v or w/w of oral formulation; at least 60% w/v or w/w of oral formulation; at least 70% w/v or w/w of oral formulation; at least 80% w/v or w/w of oral formulation; at least 90% w/v or w/w of oral formulation; at least 95% w/v or w/w of oral formulation; or at least 99% w/v or w/w of oral formulation.

In particular embodiments, the oral formulations include excipient of at least 0.1% w/v or w/w of the oral formulation; at least 1% w/v or w/w of oral formulation; at least 10% w/v or w/w of oral formulation; at least 20% w/v or w/w of oral formulation; at least 30% w/v or w/w of oral formulation; at least 40% w/v or w/w of oral formulation; at least 50% w/v or w/w of oral formulation; at least 60% w/v or w/w of oral formulation; at least 70% w/v or w/w of oral formulation; at least 80% w/v or w/w of oral formulation; at least 90% w/v or w/w of oral formulation; at least 95% w/v or w/w of oral formulation; or at least 99% w/v or w/w of oral formulation.

Excipients are commercially available from companies such as Aldrich Chemical Co., FMC Corp, Bayer, BASF, Alexi Fres, Wtco, Mallinckrodt, Rhodia, ISP, and others.

Exemplary excipient classes include binders, buffers, chelators, coating agents, colorants, complexation agents, diluents (i.e., fillers), disintegrants, emulsifiers, flavoring agents, glidants, lubricants, preservatives, releasing agents, surfactants, stabilizing agents, solubilizing agents, sweeteners, thickening agents, wetting agents, and vehicles.

Binders are substances used to cause adhesion of powder particles in granulations. Exemplary binders include acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, and polyethylene glycol.

Colorants may be included in the oral formulations to impart color to the formulation. Exemplary colorants include grape skin extract, beet red powder, beta carotene, annato, carmine, turmeric, and paprika. Additional colorants include FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide.

Diluents can enhance the granulation of oral formulations. Exemplary diluents include microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycine.

Disintegrants also may be included in the oral formulations in order to facilitate dissolution. Disentegrants, including permeabilising and wicking agents, are capable of drawing water or saliva up into the oral formulations which promotes dissolution from the inside as well as the outside of the oral formulations. Such disintegrants, permeabilising and/or wicking agents that may be used include starches, such as corn starch, potato starch, pre-gelatinized and modified starches thereof, cellulosic agents, such as Ac-di-sol, montmorrilonite clays, cross-linked PVP, sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents, such as hydroxymethylcellulose, hydroxypropylcellulose and hydroxyopropylmethylcellulose. Dissolution of the oral formulations may be facilitated by including relatively small particles sizes of the ingredients used.

Exemplary dispersing or suspending agents include acacia, alginate, dextran, fragacanth, gelatin, hydrogenated edible fats, methylcellulose, polyvinylpyrrolidone, sodium carboxymethyl cellulose, sorbitol syrup, and synthetic natural gums.

Exemplary emulsifiers include acacia and lecithin.

Flavorants are natural or artificial compounds used to impart a pleasant flavor and often odor to oral formulations. Exemplary flavorants include, natural and synthetic flavor oils, flavoring aromatics, extracts from plants, leaves, flowers, and fruits and combinations thereof. Such flavorants include anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils, such as lemon, orange, lime and grapefruit oils; and fruit essences, including apple, pear, peach, berry, wildberry, date, blueberry, kiwi, strawberry, raspberry, cherry, plum, pineapple, and apricot. In particular embodiments, flavorants that may be used include natural berry extracts and natural mixed berry flavor, as well as citric and malic acid.

Glidants improve the flow of powder blends during manufacturing and minimize oral formulation weight variation. Exemplary glidants include silicon dioxide, colloidal or fumed silica, magnesium stearate, calcium stearate, stearic acid, cornstarch, and talc.

Lubricants are substances used in oral formulations that reduce friction during formulation compression. Exemplary lubricants include stearic acid, calcium stearate, magnesium stearate, zinc stearate, talc, mineral and vegetable oils, benzoic acid, poly(ethylene glycol), glyceryl behenate, stearyl fumarate, and sodium lauryl sulfate.

Exemplary preservatives include methyl p-hydroxybenzoates, propyl p-hydroxybenzoates, and sorbic acid.

Exemplary sweeteners include aspartame, dextrose, fructose, high fructose corn syrup, maltodextrin, monoammonium glycyrrhizinate, neohesperidin dihydrochalcone, potassium acesulfame, saccharin sodium, stevia, sucralose, and sucrose.

Particular embodiments include swallowable formulations. Swallowable formulations are those that do not readily dissolve when placed in the mouth and may be swallowed whole without chewing or discomfort. U.S. Pat. Nos. 5,215,754 and 4,374,082 describe methods for preparing swallowable formulations. In particular embodiments, swallowable formulations may have a shape containing no sharp edges and a smooth, uniform and substantially bubble free outer coating.

To prepare swallowable formulations, each of the ingredients may be combined in intimate admixture with a suitable carrier according to conventional compounding techniques. In particular embodiments of the swallowable formulations, the surface of the formulations may be coated with a polymeric film. Such a film coating has several beneficial effects. First, it reduces the adhesion of the formulations to the inner surface of the mouth, thereby increasing the subject's ability to swallow the formulations. Second, the film may aid in masking the unpleasant taste of certain ingredients. Third, the film coating may protect the formulations from atmospheric degradation. Polymeric films that may be used in preparing the swallowable formulations include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia.

In particular embodiments, the oral formulations may include chewable formulations. Chewable formulations are those that have a palatable taste and mouthfeel, are relatively soft and quickly break into smaller pieces and begin to dissolve after chewing such that they are swallowed substantially as a solution.

U.S. Pat. No. 6,495,177 describes methods to prepare chewable formulations with improved mouthfeel. U.S. Pat. No. 5,965,162, describes kits and methods for preparing comestible units which disintegrate quickly in the mouth, especially when chewed.

In order to create chewable formulations, certain ingredients should be included to achieve the attributes just described. For example, chewable formulations should include ingredients that create pleasant flavor and mouthfeel and promote relative softness and dissolvability in the mouth. The following discussion describes ingredients that may help to achieve these characteristics.

Sugars such as white sugar, corn syrup, sorbitol (solution), maltitol (syrup), oligosaccharide, isomaltooligosaccharide, sucrose, fructose, lactose, glucose, lycasin, xylitol, lactitol, erythritol, mannitol, isomaltose, dextrose, polydextrose, dextrin, compressible cellulose, compressible honey, compressible molasses and mixtures thereof may be added to improve mouthfeel and palatability. Fondant or gums such as gelatin, agar, arabic gum, guar gum, and carrageenan may be added to improve the chewiness of the formulations. Fatty materials that may be used include vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° to 42° C.), Cacao fat, margarine, butter, and shortening.

Alkyl polysiloxanes (commercially available polymers sold in a variety of molecular weight ranges and with a variety of different substitution patterns) also may be used to enhance the texture, the mouthfeel, or both of chewable formulations. By “enhance the texture” it is meant that the alkyl polysiloxane improves one or more of the stiffness, the brittleness, and the chewiness of the chewable formulation, relative to the same preparation lacking the alkyl polysiloxane. By “enhance the mouthfeel” it is meant that the alkyl polysiloxane reduces the gritty texture of the chewable formulation once it has liquefied in the mouth, relative to the same preparation lacking the alkyl polysiloxane.

Alkyl polysiloxanes generally include a silicon and oxygen-containing polymeric backbone with one or more alkyl groups pending from the silicon atoms of the back bone. Depending upon their grade, they can further include silica gel. Alkyl polysiloxanes are generally viscous oils. Exemplary alkyl polysiloxanes that can be used in swallowable, chewable or dissolvable formulations include monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation which contains 30% simethicone USP. Simethicone USP contains not less than 90.5% by weight (CH₃)₃—Si{OSi(CH₃)₂}CH₃ in admixture with 4.0% to 7.0% by weight SiO₂.

To prevent the stickiness that can appear in some chewable formulations and to facilitate conversion of the active ingredients to emulsion or suspension upon taking, the formulations, may further include emulsifiers such as glycerin fatty acid ester, sorbitan monostearate, sucrose fatty acid ester, lecithin and mixtures thereof. In particular embodiments, one or more of such emulsifiers may be present in an amount of 0.01% to 5.0%, by weight of the oral formulations. If the level of emulsifier is lower or higher, in particular embodiments, an emulsification cannot be realized, or wax value will rise.

Liquid preparations for oral administration may take the form of, for example, solutions, syrups or suspensions, or they may be presented as a dry product for reconstitution with water or other suitable vehicles before use.

In addition to those described above, any appropriate fillers and excipients may be utilized in preparing the swallowable, chewable and/or dissolvable formulations or any other oral formulation described herein so long as they are consistent with the described objectives.

Oral formulations also include edibles. Edibles refer to any product that can be consumed as a food or a drink. In some cases, edibles can be made by infusion of the formulation provided herein into a foodstuff. Examples of edible foods appropriate for use include candy, a candy bar, bread, a brownie, cake, cheese, chocolate, cocoa, a cookie, gummy candy, a lollipop, a mint, a pastry, peanut butter, popcorn, a protein bar, rice cakes, yogurt, etc. While technically not edible, gums can also be used. Examples of edible drinks include beer, juice, flavored milk, flavored water, liquor, milk, punch, a shake, soda, tea, and water. In particular embodiments, edibles are made by combining a synthetic cannabinoid/carrier formulation with ingredients used to make an edible. Examples include butters and oils. Exemplary oils include coconut oil, grape seed oil, olive oil, palm oil, papaya seed oil, peanut oil, sesame oil, sprouted wheat oil, wheat germ oil, or any combination thereof.

Oral formulations can be individually wrapped or packaged as multiple units in one or more packages, cans, vials, blister packs, or bottles of any size. Doses are sized to provide therapeutically effective amounts.

Additional information can be found in WADE & WALLER, HANDBOOK OF PHARMACEUTICAL EXCIPIENTS (2nd ed. 1994) and Remington's Pharmaceutical Sciences, 18th Ed. Mack Printing Company, 1990. Moreover, formulations can be prepared to meet sterility, pyrogenicity, general safety, and purity standards as required by U.S. FDA and/or other relevant foreign regulatory agencies.

Particular embodiments include a rapid onset layer including vegetable matter and/or synthetic cannabinoids, a filler, a disintegrating agent, and an additive; and an extended action component including vegetable matter and/or synthetic cannabinoids, a filler, a disintegrating agent, release-controlling matrix, and an additive.

Particular embodiments include an extended action core, a first extended action enteric coating and a rapid onset shell. The extended action core can include vegetable matter and/or synthetic cannabinoids, and excipients, notably a lubricant, and a binder and/or a filler, and optionally a glidant.

Plant-based formulations disclosed herein can be used to treat subjects (humans, veterinary animals (dogs, cats, reptiles, birds, etc.), livestock (horses, cattle, goats, pigs, chickens, etc.), and research animals (monkeys, rats, mice, fish, etc.)). Treating subjects includes providing therapeutically effective amounts. Therapeutically effective amounts include those that provide effective amounts, prophylactic treatments, and/or therapeutic treatments.

An “effective amount” is the amount of a plant-based formulation necessary to result in a desired physiological change in a subject. Effective amounts are often administered for research purposes. Representative effective amounts disclosed herein can reduce pain perception in an animal model (neuropathic pain, acute pain, visceral pain), stimulate appetite in an animal model, reduce seizures (e.g., epileptic seizures) in an animal model, reverse bone loss in an animal model, relieve migraine (vasoconstrict cranial blood vessels) in an animal model, treat addiction in an animal model, reduce anxiety in an animal model, and/or reduce symptoms of asthma in an animal model.

A “prophylactic treatment” includes a treatment administered to a subject who does not display signs or symptoms of a disease or nutritional deficiency, or displays only early signs or symptoms of a disease or nutritional deficiency, such that treatment is administered for the purpose of diminishing, preventing, or decreasing the risk of developing the disease or nutritional deficiency further. Thus, a prophylactic treatment functions as a preventative treatment against the development of diseases or nutritional deficiencies.

As one example of a prophylactic treatment, an oral formulation disclosed herein can be administered to a subject who is at risk of developing a migraine headache. An effective prophylactic treatment of a migraine headache occurs when the number of migraines per month experienced by a subject is reduced by at least 10% or in particular embodiments, by 25%.

As another example of a prophylactic treatment, an oral formulation disclosed herein can be administered to a subject who is at risk of having an epileptic seizure. An effective prophylactic treatment of epileptic seizures occurs when the number of seizures per month is reduced by at least 10% or in particular embodiments, by 25%.

As another example of a prophylactic treatment, an oral formulation disclosed herein can be administered to a subject who is at risk of suffering from neuropathic pain. An effective prophylactic treatment of neuropathic pain occurs when the occurrence of the neuropathic pain is reduced by at least 10%, or in particular embodiments, by 25% as measured by a standard subjective or objective pain assessment.

As another example of a prophylactic treatment, an oral formulation disclosed herein can be administered to a subject who is at risk of developing breakthrough pain. An effective prophylactic treatment of breakthrough pain occurs when the occurrence of breakthrough pain is reduced by 10%, and in particular embodiments, by 25% by a standard subjective or objective pain assessment.

As another example of a prophylactic treatment, an oral formulation disclosed herein can be administered to a subject who is at risk of developing chemotherapy induced nausea and vomiting (CINV). An effective prophylactic treatment of CINV occurs when CINV is reduced by 10%, and in particular embodiments, by 25% measured by a standard subjective or objective CINV assessment.

As an example of a prophylactic treatment of a nutritional deficiency, an oral formulation disclosed herein can be administered to a subject who is at risk of developing rickets from insufficient vitamin C, anemia from insufficient dietary iron, and/or bone loss from insufficient calcium. An effective prophylactic treatment of these conditions occurs when the conditions are avoided or delayed due to nutritional supplementation with an oral formulation disclosed herein.

A “therapeutic treatment” includes a treatment administered to a subject who has a disease or nutritional deficiency and is administered to the subject for the purpose of curing or reducing the severity of the disease or nutritional deficiency.

As one example of a therapeutic treatment, an oral formulation disclosed herein can be administered to a subject who has a migraine headache. An effective therapeutic treatment of the migraine headache occurs when the severity of the headache is reduced or relieved completely and/or the headache resolves more quickly measured by a standard subjective or objective headache assessment.

Another example of a therapeutic treatment includes administration of an oral formulation disclosed herein to a subject experiencing CINV. A therapeutic treatment of CINV occurs when the vomiting is reduced or ceases (or ceases more quickly) and the nausea is relieved measured by a standard subjective or objective CINV assessment.

Another example of a therapeutic treatment, includes administration of an oral formulation disclosed to a subject who has osteoporosis. An effective therapeutic treatment of osteoporosis occurs when bone density has increased by 10% and in particular embodiments, by 25%.

Another example of a therapeutic treatment includes administration of an oral formulation disclosed herein to a subject who has anxiety. An effective therapeutic treatment of anxiety occurs when the severity of the anxiety is reduced or relieved completely and/or more quickly measured by a standard subjective or objective anxiety assessment.

Another example of a therapeutic treatment includes administration of an oral formulation disclosed herein to a subject who has multiple sclerosis. An effective therapeutic treatment of multiple sclerosis occurs when the score in a standard walk test improves by 10% and in particular embodiments, by 25%.

As one example of a therapeutic treatment of a nutritional deficiency, an oral formulation disclosed herein can be administered to a subject who has rickets from insufficient vitamin C, anemia from insufficient dietary iron, and/or bone loss from insufficient calcium. An effective therapeutic treatment of these conditions occurs when the conditions are reduced or resolved due to nutritional supplementation with an oral formulation disclosed herein.

Therapeutic treatments can be distinguished from effective amounts based on the presence or absence of a research component to the administration. As will be understood by one of ordinary skill in the art, however, in human clinical trials effective amounts, prophylactic treatments and therapeutic treatments can overlap.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by the subject, a physician, veterinarian, or researcher taking into account parameters such as physical, physiological and psychological factors including target, body weight, condition, previous or concurrent therapeutic interventions, and/or idiopathy of the subject.

Useful doses can range from 0.1 to 5 μg/kg or from 0.5 to 1 μg/kg. In other non-limiting examples, a dose can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 to 5 mg/kg or from 0.5 to 1 mg/kg. In other non-limiting examples, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, or more.

In particular embodiments, useful doses include weight of vegetable matter or synthetic cannabinoid per body weight of a subject. In particular embodiments, useful doses can range from 0.1 mg/kg to 100 mg/kg or from 0.5 mg/kg to 50 mg/kg. In particular embodiments, useful doses include 0.5 mg/kg, 1 mg/kg, 5 mg/kg, 10 mg/kg, 15 mg/kg, 20 mg/kg, 25 mg/kg, or more of vegetable matter or synthetic cannabinoid per body weight of a subject.

In particular embodiments, useful doses include weight of carrier (e.g., SNAC) per body weight of a subject. In particular embodiments, useful doses can range from 0.1 mg/kg to 100 mg/kg or from 0.5 mg/kg to 50 mg/kg. In particular embodiments, useful doses include 0.5 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, or more of carrier per body weight of a subject.

In particular embodiments, total dose volume can range from 0.25 mL to 30 mL or from 0.5 mL to 20 mL. In particular embodiments, a total dose volume can include 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, or more.

Dose concentration can be expressed as weight of vegetable matter (e.g., plant parts or extracts) or active ingredient per dose volume (e.g., mg active pharmaceutical ingredient (API)/mL). In particular embodiments, dose concentration can range from 1 mg/mL to 100 mg/mL or from 5 mg/mL to 50 mg/mL. In particular embodiments, a dose concentration can include 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, or more.

Dose concentration can be expressed as weight of carrier (e.g., SNAC) per dose volume (e.g., mg SNAC/mL). In particular embodiments, dose concentration can range from 1 mg/mL to 500 mg/mL or from 50 mg/mL to 300 mg/mL. In particular embodiments, a dose concentration can include 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, 400 mg/mL, 425 mg/mL, 450 mg/mL, 475 mg/mL, 500 mg/mL, or more.

In particular embodiments, the ratio of carrier to vegetable matter (e.g., plant parts or extracts) or active ingredient (w/w) can range from 1:1 to 100:1 or from 1:1 to 20:1. In particular embodiments, the ratio can include 1:1, 2:1, 3:1, 4:1, 5:1, 6:1, 7:1, 8:1, 9:1, 10:1, 11:1,12:1,13:1, 14:1, 15:1, 16:1, 17:1, 18:1, 19:1, 20:1, 25:1, 30:1, 35:1, 40:1, 45:1, 50:1, 55:1, 60:1, 65:1, 70:1, 75:1, 80:1, 85:1, 90:1, 95:1, 100:1, or more. In particular embodiments, the ratio can be 10:1.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly).

One or more active agent(s) can be administered simultaneously or within a selected time window, such as within 10 minutes, 1 hour, 3 hour, 10 hour, 15 hour, 24 hour, or 48 hour time windows or when the complementary active agent(s) is within a clinically-relevant therapeutic window.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure. Exemplary Embodiments.
1. An oral formulation including (i) a rapid onset component including (a) vegetable matter and/or synthetic cannabinoids and (b) an N-acylated fatty amino acid or a salt thereof; and (ii) an extended action component including (a) vegetable matter and/or synthetic cannabinoids and (b) a sustained release system.
2. An oral formulation of embodiment 1 wherein the rapid onset component includes a liquid.
3. An oral formulation of embodiment 1 wherein the liquid includes water, ethanol, polyethylene glycols and polyvinyl alcohols.
4. An oral formulation of embodiment 1 or 2 wherein the extended action component includes particles within the liquid.
5. An oral formulation of embodiment 4 wherein the particles include a release-controlling matrix, an enteric coating and/or a barrier layer.
6. An oral formulation of embodiment 5 wherein the particles include a release-controlling matrix selected from acrylic polymers, alkyl celluloses, vegetable oils and waxes.
7. An oral formulation of embodiment 5 or 6 including a release-controlling matrix and an enteric coating.
8. An oral formulation of embodiment 7 wherein the enteric coating is selected from acrylic polymers, celluloses, phthalate polymers and resins.
9. An oral formulation of embodiment 5 or 6 including a release-controlling matrix and a barrier layer.
10. An oral formulation of embodiment 9 wherein the barrier layer is selected from gellable polymers, natural gels, swellable polymers and erodible/slow dissolving polymers and gels.
11. An oral formulation of embodiment 1 wherein the extended action component includes a liquid surrounded by the rapid onset component.
12. An oral formulation of embodiment 11 wherein the liquid is a vegetable oil.
13. An oral formulation of embodiment 11 or 12 wherein the liquid is within a gelcap.
14. An oral formulation of embodiment 13 wherein the gelcap is coated with a rapid onset shell.
15. An oral formulation of embodiment 1 in the form of a tablet.
16. An oral formulation of embodiment 15 wherein the rapid onset component of the tablet surrounds the extended action component of the tablet.
17. An oral formulation of embodiment 15 or 16 wherein the rapid onset component includes N-acylated fatty amino acid or a salt thereof, EDTA, citric acid, bile salts, chitosan, SNAC, NAC, SDS, medium chain fatty acids and acyklcarnitines.
18. An oral formulation of any of embodiments 15-17 wherein the extended action component includes a release-controlling matrix, an enteric coating and/or a barrier layer.
19. An oral formulation of embodiment 18 wherein the extended action component includes a release-controlling matrix selected from acrylic polymers, alkyl celluloses, vegetable oils and waxes.
20. An oral formulation of embodiment 18 or 19 including a release-controlling matrix and an enteric coating.
21. An oral formulation of embodiment 20 wherein the enteric coating is selected from acrylic polymers, celluloses, phthalate polymers and resins.
22. An oral formulation of embodiment 20 or 21 including a release-controlling matrix and a barrier layer.
23. An oral formulation of embodiment 22 wherein the barrier layer is selected from gellable polymers, natural gels, swellable polymers and erodible/slow dissolving polymers and gels.
24. An oral formulation of any of embodiments 1-23 including an acrylic polymer selected from one or more of acrylic acid and methacrylic acid copolymer, aminoalkyl methacrylate copolymer, cyanoethyl methacrylate, ethoxyethyl methacrylate, glycidyl methacrylate copolymer, methacrylic acid alkylamide copolymer, methyl methacrylate copolymer, poly(acrylic acid), poly(methacrylic acid), poly(methacrylic acid anhydride), poly(methyl methacrylate), poly(methyl methacrylate) copolymer, polyacrylamide, and polymethacrylate, methyl methacrylate.
25. An oral formulation of any of embodiments 1-24 including an alkyl cellulose selected from methyl cellulose and/or ethyl cellulose.
26. An oral formulation of any of embodiments 1-25 including vegetable oil selected from one or more of sesame oil, palm oil, palm hydrogenated oil, corn germ hydrogenated oil, castor hydrogenated oil, cotton-seed oil, olive oil, peanut oil, palm olein oil, and palm stearin oil.
27. An oral formulation of any of embodiments 1-26 including a wax selected from one or more of beeswax, glycowax, castor wax, and carnauba wax.
28. An oral formulation of any of embodiments 1-27 including a cellulose selected from one or more of hydroxyalkyl cellulose, methyl cellulose, ethyl cellulose, hydroxyethyl cellulose, hydroxypropyl cellulose, hydroxypropylmethyl cellulose (HPMC), hydroxypropylethyl cellulose, hydroxypropylpropyl cellulose, and hydroxypropylbutyl cellulose.
29. An oral formulation of any of embodiments 1-28 including a phthalate polymer selected from one or more of cellulose acetate hexahydrophthalate, cellulose acetate phthalate (CAP), cellulose propionate phthalate, hydroxypropyl methylcellulose hexahydrophthalate, hydroxypropyl methylcellulose phthalate (HPMCP), and polyvinyl acetate phthalate (PVAP).
30. An oral formulation of any of embodiments 1-29 including a resin selected from one or more of zein, gelatin, shellac and acacia.
31. An oral formulation of any of embodiments 1-30 including a gellable polymer selected from one or more of guar gum, mannose sugar, galactose sugar, hydropropyl guar, carboxymethyl guar, carboxymethylhydroxypropyl guar, hydroxyethylcellulose, hydroxypropylcellulose, carboxymethyl hydroxyethylcellulose, xanthan, diutan, scleroglucan, polyacrylamide, polyvinyl alcohol, polyethylene glycol, polypropylene glycol, and a polyacrylate polymer.
32. An oral formulation of any of embodiments 1-30 including a swellable polymer including one or more of poly(acrylic acid), poly(alkylene oxide), poly(vinyl alcohol), poly(vinyl pyrrolidone), polyurethane hydrogel, maleic anhydride polymer, cellulose, hydroxymethylcellulose, hydroxyethyl cellulose, hydroxypropyl cellulose, hydroxypropylmethyl cellulose, carboxymethyl cellulose, dextran, xanthan gum, gellan gum, welan gum, rhamsan gum, sodium alginate, calcium alginate, chitosan, gelatin, maltodextrin, starch, hydrolyzed starch polyacrylonitrile graft copolymers, and/or starch-acrylate-acrylamide copolymers.
33. An oral formulation of any of embodiments 1-30 including an erodible polymer including one or more of polyethylene oxide, polyethylene oxide water soluble resins, glyceryl fatty acid esters, hydrogenated castor oil, hydroxyethyl cellulose, hydroxypropyl cellulose, hydroxypropyl methylcellulose, ethylhydroxy ethylcellulose, methylethyl cellulose, carboxymethyl cellulose, carboxymethyl ethylcellulose, pullulan, polyvinyl pyrrolidone, polyvinyl alcohol, and polyvinyl acetate.
34. An oral formulation of any of embodiments 1-33 including a botanical product.
35. An oral formulation of any of embodiments 1-34 wherein the vegetable matter is derived from Calophyllum brasiliense, Calophyllum caledonicurn, Calophyllum inophyllum, Calophyllum soulattri, Uncaria tomentosa, Thymus vulgaris, Matricaria recutita, Salix alba, Calendula officinalis, Usnea barbata, Ligusticum porterii-osha, Gaultheria procumbens, Camellia sinensis, Vaccinium myrtillus, Melissa officinalis, Allium sativum, Camellia sinensis, Krameria triandra, Punica granatum, Viburnum plicatum, Nicotiana tabacum, Duboisia hopwoodii, Asclepias syriaca, Curcumalonga, Hypericum perforatum, Polygonum cuspidatum, Vitis spp., Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., Mentha spp., Cannabis sativa, Cannabis indica, Cannabis ruderalis and/or Acer spp, or an extract thereof.
36. An oral formulation of any of embodiments 1-35 wherein the vegetable matter is derived from cannabis.
37. An oral formulation of any of embodiments 1-36 wherein the vegetable matter is derived from Cannabis sativa, Cannabis ruderalis, or Cannabis indica.
38. An oral formulation of any of embodiments 1-37 including a cannabis extract.
39. An oral formulation of any of embodiments 1-38 including cannabinoids.
40. An oral formulation of any of embodiments 1-39 including Δ9-Tetrahydrocannabinol (THC) and cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), and/or mixtures thereof.
41. An oral formulation of any of embodiments 1-40 including curcumin, hypericin, resveratrol, capsaicin, reserpine, vinblastine, hesperidin, naringin, rutin, quercitrin, catechin eugenol, limonene, linalool or nicotine.
42. The oral formulation of any of embodiments 1-41 wherein the one or more synthetic cannabinoids include THC, CBD, CBG, CBC, CBN, CBDL, CBL, CBV, THCV, CBDV), CBCV, CBGV, CBGM, cannabinerolic acid, CBDA, CBNV, CBO, THCA, THCVA, or mixtures thereof.
43. The oral formulation of any of embodiments 1-42 wherein the one or more synthetic cannabinoids include a derivative and/or analog of a synthetic cannabinoid of embodiment 42 or a mixture thereof.
44. The oral formulation of any of embodiments 1-43 wherein the one or more synthetic cannabinoids include 3-carbamoyl-2-pyridone or its derivatives and/or analogs; pyrimidine derivatives and/or analogs; carenadiol or its derivatives and/or analogs; cannabinoid carboxylic acids or their derivatives and/or analogs; pyrido[3,2-E][1,2,4]triazolo[4,3-C]pyrimidine or its derivatives and/or analogs; tetrahydro-pyrazolo[3,4-C] pyridine or its derivatives and/or analogs; bicyclo[3.1.1]heptan-2-one cannabinoid or its derivatives and/or analogs; resorcinol or its derivatives and/or analogs; dexanbinol compounds or their derivatives and/or analogs; cannabimimetic lipid amide compounds or their derivatives and/or analogs; nabilone or its derivatives and/or analogs; 2-oxoquinolone compounds or their derivatives and/or analogs disclosed; or 3,4-diaryl-4,5-dihydro-(h)-pyrazole-1-carboxamide or its derivatives and/or analogs.
45. An oral formulation of any of embodiments 1-44 including flavonoid compounds, terpenes, or terpenoids.
46. An oral formulation of any of embodiments 1-45 wherein the N-acylated fatty amino acid includes one or more of Compounds I-XXXV (FIG. 5), or Compounds a-r (FIG. 6).
47. An oral formulation of any of embodiments 1-46 wherein the N-acylated fatty amino acid includes monosodium-N-salicyloyl-8-aminocaprylate, disodium-N-salicyloyl-8-aminocaprylate, and N-(salicyloyl)-8-aminocaprylic acid.
48. An oral formulation of any of embodiments 1-47 wherein the N-acylated fatty amino acid or a salt thereof includes

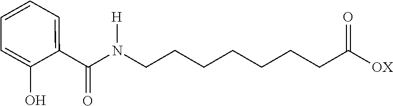

wherein X and Z are independently H, a monovalent cation, a divalent metal cation, or an organic cation.
49. An oral formulation of embodiment 48 wherein the monovalent cation includes sodium or potassium.
50. An oral formulation of embodiment 48 or 49 wherein the metal cation includes calcium or magnesium.
51. An oral formulation of any of embodiments 48-50 wherein the organic cation includes ammonium or tetramethylammonium.
52. An oral formulation of any of embodiments 48-51 wherein X is H.
53. An oral formulation of any of embodiments 48-51 wherein X is a monovalent cation including sodium or potassium.
54. An oral formulation of any of embodiments 48-51 wherein X is a divalent metal cation including calcium or magnesium.
55. An oral formulation of any of embodiments 48-51 wherein X is an organic cation including ammonium or tetramethylammonium.
56. An oral formulation of any of embodiments 48-55 wherein Z is H.
57. An oral formulation of any of embodiments 48-55 wherein Z is a monovalent cation comprising sodium or potassium.
58. An oral formulation of any of embodiments 48-55 wherein Z is a divalent cation comprising calcium or magnesium.
59. An oral formulation of embodiment 48 wherein X is H and Z is H.
60. An oral formulation of embodiment 48 wherein X is H and Z is sodium.
61. An oral formulation of embodiment 48 wherein X is sodium and Z is sodium.
62. An oral formulation of any of embodiments 1-61 wherein the N-acylated fatty amino acid provides an administration benefit.
63. An oral formulation of embodiment 63 wherein the administration benefit is a dose-dependent administration benefit.
64. An oral formulation of embodiment 63 wherein the dose-dependent administration benefit is at a dose of 100-200 mg.
65. An oral formulation of any of embodiments 62-64 wherein the administration benefit includes one or more of increased absorption of a measured component of vegetable matter, increased bioavailability of a measured component of vegetable matter, faster onset of action of a measured component of vegetable matter, higher peak concentrations of a measured component of vegetable matter, faster time to peak concentrations of a measured component of vegetable matter, increased subjective therapeutic efficacy, increased objective therapeutic efficacy, improved taste, and improved mouthfeel as compared to a control oral formulation without the N-acylated fatty amino acid.
66. An oral formulation of any of embodiments 1-65 wherein the oral formulation is a medicinal oral formulation.
67. An oral formulation of any of embodiments 1-65 wherein the oral formulation is a nutritional supplement.
68. An oral formulation of any of embodiments 1-67 including a surfactant, detergent, azone, pyrrolidone, glycol or bile salt.
69. An oral formulation of any of embodiments 1-66, or 68 including a therapeutically effective amount of vegetable matter.
70. An oral formulation of embodiment 69 wherein the therapeutically effective amount treats a symptom of acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Grave's disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, and withdrawal.
71. An oral formulation of any of embodiments 1-70 including vitamins or minerals.
72. An oral formulation of any of embodiments 1-70 including vitamins and minerals.
73. An oral formulation of embodiment 71 or 72 wherein the vitamins are selected from one or more of Vitamin A, Vitamin B1, Vitamin B6, Vitamin B12, Vitamin C, Vitamin D, Vitamin E, or Vitamin K.
74. An oral formulation of any of embodiments 71-73 wherein the minerals are selected from one or more of calcium, chromium, iodine, iron, magnesium, selenium or zinc.
75. An oral formulation of embodiment 1-74 wherein the oral formulation is swallowable or chewable.
76. An oral formulation of any of embodiment 1-75 wherein the oral formulation is liquid or solid.
77. An oral formulation of any of embodiments 1-76 wherein the oral formulation is a solution, suspension, or spray.
78. An oral formulation of any of embodiments 1-76 wherein the oral formulation is a tablet, capsule or sachet.
79. An oral formulation of any of embodiments 1-78 wherein the oral formulation is flavored.
80. A method of preparing an oral formulation of cannabis having a faster onset of action, wherein the method comprises adding an absorption enhancer to the oral formulation of cannabis and wherein the oral formulation of cannabis has a faster onset of action than an oral formulation of cannabis without an absorption enhancer.
81. The method of embodiment 80, wherein the absorption enhancer is an N-acylated fatty amino acid or a salt thereof.
82. The method of embodiment 81, wherein the N-acylated fatty amino acid or a salt thereof comprises

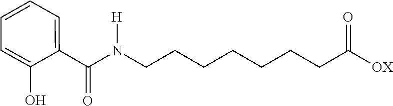

wherein X and Z are independently H, a monovalent cation, a divalent metal cation, or an organic cation.
83. The method of embodiment 81 or 82 wherein the N-acylated fatty amino acid is selected from monosodium-N-salicyloyl-8-aminocaprylate, disodium-N-salicyloyl-8-aminocaprylate, and N-(salicyloyl)-8-aminocaprylic acid.
84. A method of treating a subject in need thereof including administering a therapeutically effective amount of an oral formulation of any of embodiments 1-66, or 68-79 to the subject thereby treating the subject in need thereof.
85. A method of embodiment 84 wherein the therapeutically effective amount provides an effective amount, a prophylactic treatment, and/or a therapeutic treatment.
86. A method of reducing or eliminating one or more symptoms of a disease or disorder in a human subject,
   wherein said method includes delivering a therapeutically effective amount of an oral formulation of any of embodiments 1-66, or 68-79 to the subject, thereby reducing or eliminating one or more symptoms of the disease or disorder, and
   wherein said disease or disorder is acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Grave's disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.

### EXAMPLES

Oral cannabinoid dosage form providing improved bioavailability and shortened time to onset of effect. Considering the wealth of medical conditions potentially benefiting from cannabis therapy, a significant unmet need exists for a faster-acting product that provides improved bioavailability in an oral format. Current oral cannabis products include edibles and traditional pharmaceutical dosage forms that are challenged by low bioavailability, and prolonged time to onset of action. The present disclosure addresses the shortcomings of all of the currently available oral cannabis products to provide an improved time to onset of effect and improved bioavailability.

### Example 1

Exemplary Formulations. Rapid Onset Solution Formulation. Cannabis and one or more N-acylated fatty amino acids are combined in an aqueous/organic solvent mixture. The resulting blend is stirred vigorously for an hour. If solution is incomplete, a surfactant can be added and stirring can be continued to prepare the final formulation.

Rapid Onset Suspension formulation. Cannabis and one or more N-acylated fatty amino acids are combined in water, an aqueous/organic solvent mixture or an organic solvent mixture. The resulting blend can be stirred to effect suspension.

Rapid Onset Solution formulation. Cannabis and one or more absorption enhancing agents are combined in an aqueous/organic solvent mixture. The resulting blend is stirred vigorously for an hour. If solution is incomplete, a surfactant can be added and stirring can be continued to prepare the final formulation.

Rapid Onset Suspension formulation. Cannabis and one or more absorption enhancing agents are combined in water, an aqueous/organic solvent mixture or an organic solvent mixture. The resulting blend can be stirred to effect suspension.

Rapid Onset Gelcap Formulation. A suspension formulation or solution formulation can be filled into a gelcap to contain up to 1 g of cannabis. The gelcap can be treated with an enteric coat or used without a coating.

Rapid Onset Tablet/capsule Formulation. The solution formulation and the suspension formulation can be dried by evaporation, lyophilization, or spray drying. The resultant dry product can be combined with tableting excipients and compressed into tablets or caplets to contain up to 1 g of cannabis. Alternatively, the dry product can be filled into capsules.

Rapid Onset/Extended Action solid dosage formulation. Cannabis and one or more N-acylated fatty amino acids are combined in an aqueous/organic solvent mixture or in a granulation process. The solvent is removed and the resulting dry powder is used to coat a solid core of cannabis and excipients. The outer coating provides rapid onset cannabis and the inner core provides extended action cannabis.

Rapid Onset/Extended Action solid dosage formulation. Cannabis and one or more N-acylated fatty amino acids are combined in an aqueous/organic solvent mixture or in a granulation process. The solvent is removed and the resulting dry powder is compressed into a tablet that forms one-half of a composite tablet. The other half of the composite tablet is a tablet comprising cannabis and excipients. The two-halves are joined to form a single tablet, one-half provides rapid cannabis onset and the other provides extended action cannabis.

Rapid Onset/Extended Action solid dosage formulation. Cannabis and one or more N-acylated fatty amino acids are combined in an aqueous/organic solvent mixture or in a granulation process. The solvent is removed and the resulting dry powder is used to coat a gelcap containing cannabis in vegetable oil. The outer coating provides rapid onset cannabis and the gelcap provides extended action cannabis.

Rapid Onset/Extended Action liquid dosage formulation. Cannabis and one or more N-acylated fatty amino acids are combined in an aqueous/organic solvent mixture. Solid particles comprising cannabis and excipients are suspended in the aqueous/organic solvent mixture. The liquid portion of the formulation provides rapid onset cannabis and the suspended solid particles provide extended action cannabis.

Rapid Onset/Extended Action liquid dosage formulation. Cannabis and one or more N-acylated fatty amino acids are combined in an aqueous/organic solvent mixture. Solid particles comprising cannabis and excipients coated with a barrier layer are suspended in the aqueous/organic solvent mixture. The liquid portion of the formulation provides rapid onset cannabis and the suspended solid particles with a barrier layer provide extended action cannabis.

Rapid Onset/Extended Action edible formulation. Cannabis and one or more N-acylated fatty amino acids are combined in an aqueous/organic solvent mixture or in a granulation process. The solvent is removed to give a dry powder, rapid onset cannabis. Cannabis and one or more excipients are combined in an aqueous/organic solvent mixture or in a granulation process. The solvent is removed to give a dry powder, extended action cannabis. The dry powders are combined in a gum or edible format (e.g. candy, cake).

Rapid Onset Shell Surrounding Extended Action Liquid. A rapidly dissolving solid shell surrounds a liquid core from vegetable matter and/or a synthetic cannabinoid dissolves slowly.

Rapid Onset Shell Surrounding Extended Action Liquid. A rapidly dissolving SNAC/cannabis shell surrounds THC in sesame oil. The sesame oil can be within a gelcap barrier layer.

### Example 2

Onset and duration of action of orally administered cannabis/SNAC Formulation. This study was designed to assess the utility of SNAC in enabling a rapid-acting oral form of cannabis.

Selection of Participants. Six study participants were recruited to ingest cannabis formulations and record the onset, duration, and intensity of cannabis-induced euphoria and/or dysphoria. Study participants took part in two separate tests: 1) use of a control substance, which included liquid cannabis extract dissolved in aqueous ethanol, and 2) use of a test substance, which included the liquid cannabis extract dissolved in aqueous ethanol, as well as SNAC.

Formulations. The selected cannabis concentrate is commercially available and was provided to participants in an ethanol solution. The concentrate contains 8 mg THC per dose. It was selected because it contains a high percentage of THC, which provides a noticeable effect on user-reported “euphoria”. Aqueous ethanol was used as solvent because it effectively dissolves cannabis extract, as well as SNAC.

Methods. For the Control experiment, each participant mixed the cannabis concentrate with 15 ml (one tablespoon) of aqueous ethanol, and immediately swallowed the mixture.

For the Test experiment, each participant mixed the cannabis concentrate with a pre-mixed solution of aqueous ethanol and 200 mg SNAC, and immediately swallowed the dissolved mixture.

For both the Control experiment and the Test experiment, each participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-10. Table 1 shows descriptions of euphoria and dysphoria levels for each scale value.

TABLE 1
Scale Values for Reporting
Euphoria and Dysphoria

| Scale | |
|---|---|
| Value | Description |
| 0 | No observed effect |
| 1-2 | Mild observed effect; |
| | possibly psychological |
| 3-4 | Definite but mild effect |
| 5-6 | Definite substantial effect |
| 7-8 | Strong effect |
| 9-10 | Intense effect |

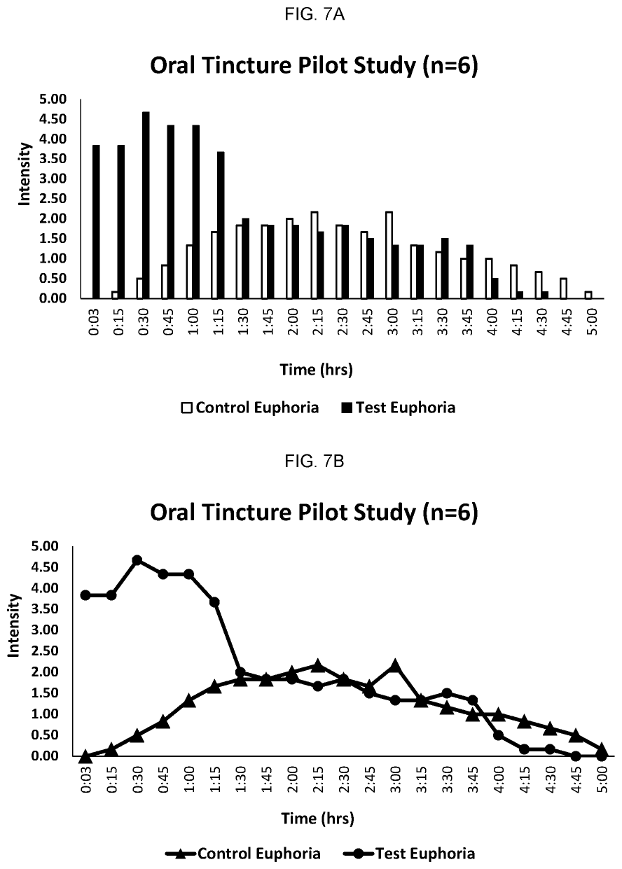

Results. The results shown below are the average scale values obtained for all six participants (also shown in FIGS. 7A and 7B).

TABLE 2
Control Experiment (n = 6)

| Actual | Time from | Observed | Observed |
|---|---|---|---|
| Time | Start | “Euphoria” | “Dysphoria” |
| 12:00 PM | 0:00 | (0-10) | (0-10) |
| 12:15 PM | 0:15 | 0.17 | 0.00 |
| 12:30 PM | 0:30 | 0.50 | 0.00 |
| 12:45 PM | 0:45 | 0.83 | 0.17 |
| 1:00 PM | 1:00 | 1.33 | 0.17 |
| 1:15 PM | 1:15 | 1.67 | 0.50 |
| 1:30 PM | 1:30 | 1.83 | 0.67 |
| 1:45 PM | 1:45 | 1.83 | 0.83 |
| 2:00 PM | 2:00 | 2.00 | 0.50 |
| 2:15 PM | 2:15 | 2.17 | 0.50 |
| 2:30 PM | 2:30 | 1.83 | 0.33 |
| 2:45 PM | 2:45 | 1.67 | 0.33 |
| 3:00 PM | 3:00 | 2.17 | 0.33 |
| 3:15 PM | 3:15 | 1.33 | 0.17 |
| 3:30 PM | 3:30 | 1.17 | 0.00 |
| 3:45 PM | 3:45 | 1.00 | 0.00 |
| 4:00 PM | 4:00 | 1.00 | 0.00 |
| 4:15 PM | 4:15 | 0.83 | 0.00 |
| 4:30 PM | 4:30 | 0.67 | 0.00 |
| 4:45 PM | 4:45 | 0.50 | 0.00 |
| 5:00 PM | 5:00 | 0.17 | 0.00 |

TABLE 3
Test Experiment (n = 6)

| Actual | Time from | Observed | Observed |
|---|---|---|---|
| Time | Start | “Euphoria” | “Dysphoria” |
| 12:00 PM | 0:00 | (0-10) | (0-10) |
| 12:03 PM | 0:03 | 3.83 | 0.67 |
| 12:15 PM | 0:15 | 3.83 | 0.67 |
| 12:30 PM | 0:30 | 4.67 | 0.83 |
| 12:45 PM | 0:45 | 4.33 | 0.50 |
| 1:00 PM | 1:00 | 4.33 | 0.50 |
| 1:15 PM | 1:15 | 3.67 | 0.67 |
| 1:30 PM | 1:30 | 2.00 | 0.17 |
| 1:45 PM | 1:45 | 1.83 | 0.17 |
| 2:00 PM | 2:00 | 1.83 | 0.00 |
| 2:15 PM | 2:15 | 1.67 | 0.00 |
| 2:30 PM | 2:30 | 1.83 | 0.00 |
| 2:45 PM | 2:45 | 1.50 | 0.00 |
| 3:00 PM | 3:00 | 1.33 | 0.17 |
| 3:15 PM | 3:15 | 1.33 | 0.17 |
| 3:30 PM | 3:30 | 1.50 | 1.00 |
| 3:45 PM | 3:45 | 1.33 | 0.00 |
| 4:00 PM | 4:00 | 0.50 | 0.00 |
| 4:15 PM | 4:15 | 0.17 | 0.00 |
| 4:30 PM | 4:30 | 0.17 | 0.00 |
| 4:45 PM | 4:45 | 0.00 | 0.00 |
| 5:00 PM | 5:00 | 0.00 | 0.00 |

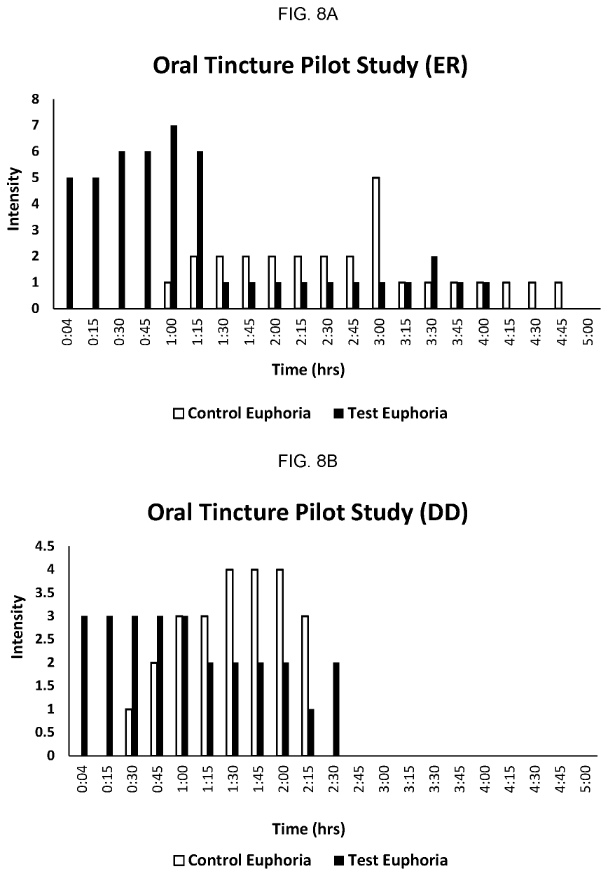

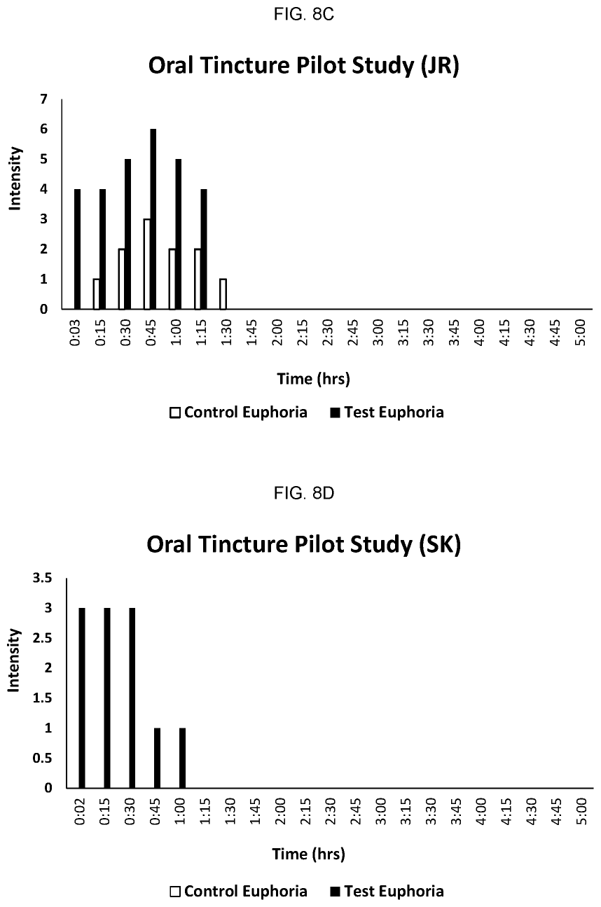

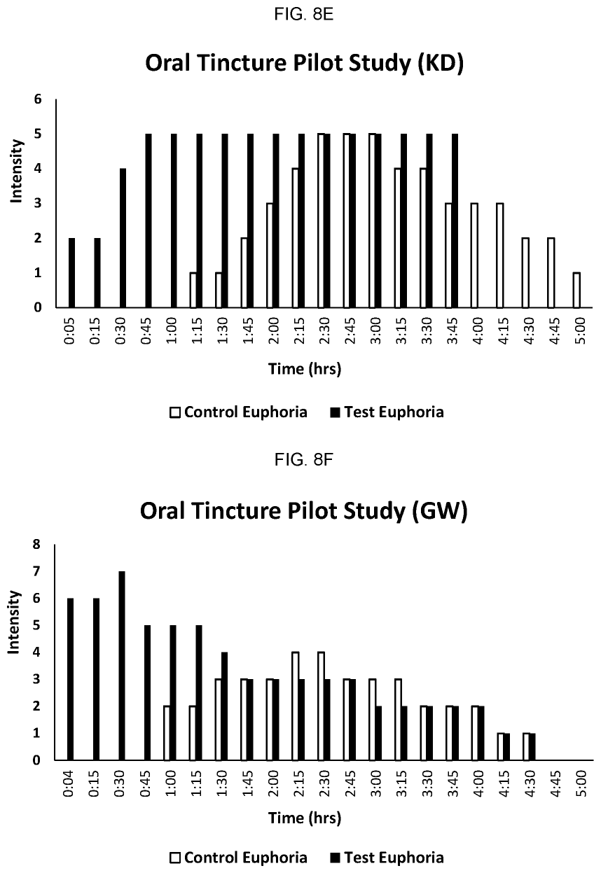

Onset: All six participants reported euphoria within five minutes of ingesting the cannabis/SNAC formulation (Test), with the time of onset ranging between two and five minutes. In contrast, the first time-point of euphoria reported by participants after ingestion of the cannabis-only formulation (Control) was fifteen minutes post-ingestion, with the time of onset ranging between fifteen minutes and one hour, fifteen minutes (see FIGS. 8A-8F for individual participant results). By fifteen minutes post-ingestion, the average reported euphoria scale value was 3.8 for the cannabis/SNAC formulation (Test). In contrast, fifteen minutes after ingestion of the cannabis-only formulation (Control), the average reported euphoria scale value was 0.17 (see FIGS. 7A, 7B for averages at each time-point).

Intensity: The average peak euphoria scale value after ingestion of the cannabis/SNAC formulation (Test) was 4.7, which occurred thirty minutes post-ingestion. In contrast, the highest average euphoria scale value after ingestion of the cannabis-only formulation (Control) was 2.2, which was at the two hour, fifteen minute time-point (see FIGS. 7A, 7B). Therefore, ingestion of the cannabis/SNAC formulation led to a higher peak intensity of euphoria, which occurred an average of one hour and forty-five minutes faster than when the cannabis-only formulation was ingested. The intensity of observed dysphoria was minimal for both the Test and Control, with a peak average scale value of 0.83 for both experiments.

Duration: The results indicate that the addition of an absorption enhancer does not shorten the duration of action of cannabis.

In summary, adding an absorption enhancer, such as SNAC, in an oral dosage formulation of cannabis provides faster onset of action and higher intensity of action at peak activity level of cannabis. Moreover, the absorption enhancer has no effect on the duration of action of cannabis.

### Example 3

Onset and duration of action of orally administered cannabis/SNAC formulation at a low SNAC dose. This study was designed to assess the utility of SNAC in enabling a rapid-acting oral form of cannabis at a low dose.

Selection of Participants. Three study participants were recruited to ingest cannabis formulations and record the onset, duration, and intensity of cannabis-induced euphoria and/or dysphoria. Study participants took part in two separate tests: 1) use of a control substance, which included liquid cannabis extract dissolved in aqueous ethanol, and 2) use of a test substance, which included the liquid cannabis extract dissolved in aqueous ethanol, as well as SNAC.

Formulations. The selected cannabis concentrate is commercially available and was provided to participants in an ethanol solution. The concentrate contains 8 mg THC per dose. It was selected because it contains a high percentage of THC, which provides a noticeable effect on user-reported “euphoria”. Aqueous ethanol was used as solvent because it effectively dissolves cannabis extract, as well as SNAC.

Methods. For the Control experiment, each participant mixed the cannabis concentrate with 15 ml (one tablespoon) of aqueous ethanol, and immediately swallowed the mixture.

For the Test experiment, each participant mixed the cannabis concentrate with a pre-mixed solution of aqueous ethanol and 100 mg SNAC, and immediately swallowed the dissolved mixture.

For both the Control experiment and the Test experiment, each participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-10. Table 1 shows descriptions of euphoria and dysphoria levels for each scale value.

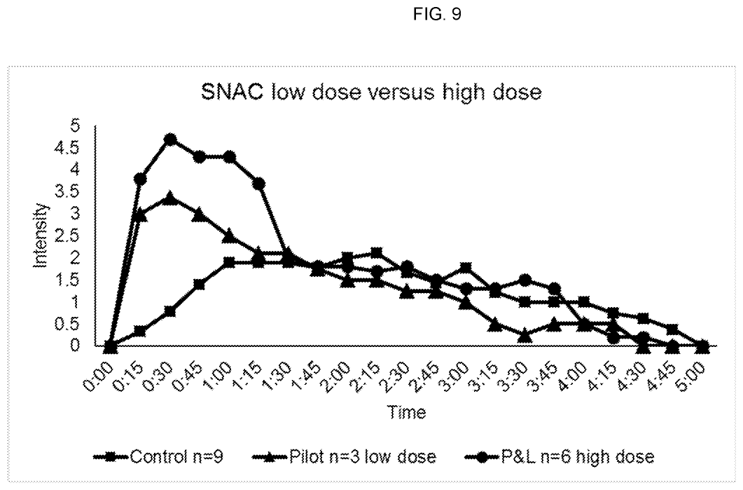

Results. The results are combined with the data from Example 2 and are reported for all participants in FIG. 9.

Onset: All three participants reported euphoria within five minutes of ingesting the cannabis/SNAC formulation (Test), with the time of onset ranging between two and five minutes. In contrast, the first time-point of euphoria reported by participants after ingestion of the cannabis-only formulation (Control) was fifteen minutes post-ingestion, with the time of onset ranging between fifteen minutes and one hour, fifteen minutes. By fifteen minutes post-ingestion, the average reported euphoria scale value was 3.0 for the cannabis/SNAC formulation (Test). In contrast, fifteen minutes after ingestion of the cannabis-only formulation (Control), the average reported euphoria scale value was 0.25.

Intensity: The average peak euphoria scale value after ingestion of the cannabis/SNAC formulation (Test) was 3.4, which occurred thirty minutes post-ingestion. In contrast, the highest average euphoria scale value after ingestion of the cannabis-only formulation (Control) was 2.2, which was at the two hour, fifteen minute time-point. Compared to Example 2 where the SNAC dose was 200 mg, the participants in Example 3 ingested only 100 mg of SNAC combined with the same quantity of cannabis used in Example 2. This reduced quantity of SNAC resulted in a reduced cannabis effect demonstrating a clear dose-response relationship between observed cannabis effect (euphoria) and SNAC dose. Consistent with Example 2, ingestion of the cannabis/SNAC formulation led to a higher peak intensity of euphoria, which occurred an average of one hour and forty-five minutes faster than when the cannabis-only formulation was ingested.

Duration: The results indicate that the addition of an absorption enhancer does not shorten the duration of action of cannabis.

In summary, adding an absorption enhancer, such as SNAC, in an oral dosage formulation of cannabis provides faster onset of action and higher intensity of action at peak activity level of cannabis. Moreover, the absorption enhancer has no effect on the duration of action of cannabis. The varying quantity of SNAC produces a clear dose-response relationship between observed cannabis effect (euphoria) and SNAC dose.

### Example 4

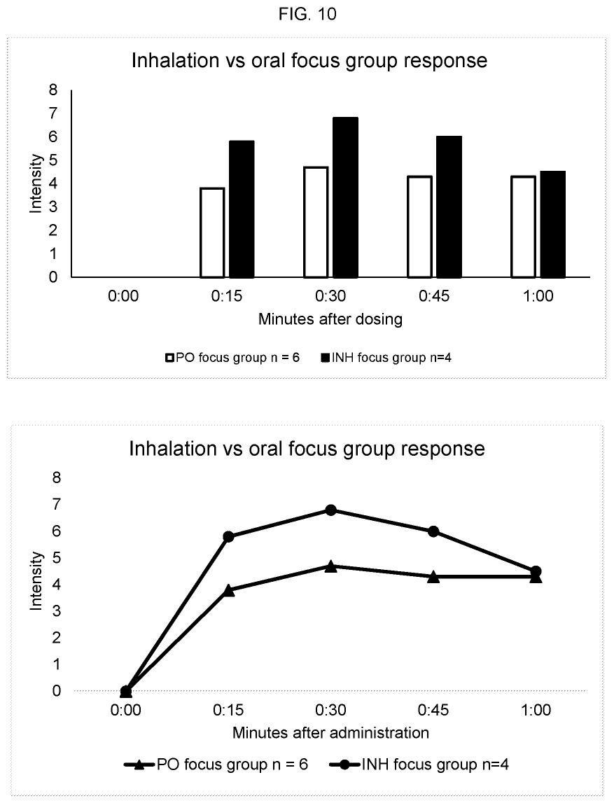

Inhalation versus oral group response (FIG. 10). Comparison of the pharmacodynamic response to inhaled and oral cannabis measured as subject-reported euphoria. Both the oral and inhaled groups reported similar time to peak effect (15-30 minutes). This is very surprising because oral cannabis is traditionally characterized by a very slow time to peak effect (up to 4 hours).

### Example 5

Summary of cannabis/SNAC oral rat pharmacokinetic (PK) study. The study was designed to characterize the pharmacokinetic profile of cannabis extract containing 56% THC/CBD in a 1:1 ratio (by weight) with and without the excipient, SNAC, following a single oral gavage administration to rats. In this study two doses of cannabis and SNAC and two ratios of cannabis to SNAC were tested. The experimental design is presented in Table 4 below.

TABLE 4
Experimental design.

| | | Extract¹ | SNAC | | | Dose | |
|---|---|---|---|---|---|---|---|
| | | Dose | Dose | Dose | Dose | Concentration³ | Number |
| Group | Group | Level | Level | Volume | Concentration² | (mg | of |
| Nos | Designation | (mg/kg) | (mg/kg) | (mL/kg) | (mg API/mL) | SNAC/mL) | Males |
| 1 | Excipient | 0 | 500 | 2 | 0 | 250 | 6 |
| | Control | | | | | | |
| 2 | Cannabis | 25 | 0 | 2 | 12.5 | 0 | 6 |
| | Control | | | | | | |
| 3 | Low Dose | 25 | 250 | 2 | 12.5 | 125 | 6 |
| 4 | Mid Dose | 25 | 500 | 2 | 12.5 | 250 | 6 |
| 5 | High Dose | 50 | 500 | 2 | 25 | 250 | 6 |

¹Extract contains 54% by weight (27% THC + 27% CBD) as the API (Active Pharmaceutical Ingredient)
²Dose of cannabis extract contains a mixture of THC:CBD in a ratio of 1:1 by weight
³SNAC dose is 10 times (THC + CBD) dose for groups 3 and 5 and 20 times for group 4.

Methods. Animals were dosed on Day 1 and a series of blood samples were collected over a period of 4 hours post dose for pharmacokinetic evaluation. Animals were euthanized following collection of their last blood sample.

Results. Following a single oral administration of cannabis extract containing THC/CBD in a 1:1 ratio combined with the absorption enhancing excipient (SNAC) at 25 mg extract/kg and 250 mg SNAC/kg (Group 3), 25 mg extract/kg and 500 mg SNAC/kg (Group 4), or 50 mg extract/kg and 500 mg SNAC/kg (Group 5), mean maximum concentration Cₘₐₓ ranged from 31.7 to 159.3 ng/mL for CBD and from 111.5 to 546.17 ng/mL for THC. The time to reach the mean maximum plasma concentration (Tₘₐₓ) ranged from 0.25 to 1 hour post dose for CBD and was reached at 1 hour post dose for the low and mid dose groups and at 2 hours post dose for the high dose group for THC. The AUC_{0-Tlast} ranged from 13.17 to 382.14 hr*ng/mL for CBD and from 170.64 to 1256.49 hr*ng/mL for THC.

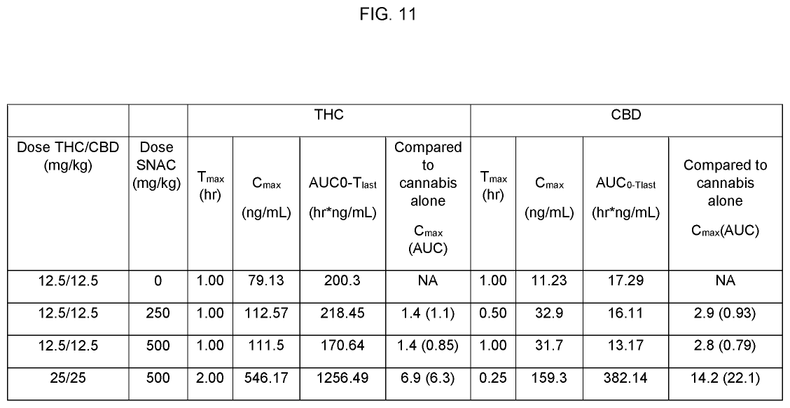

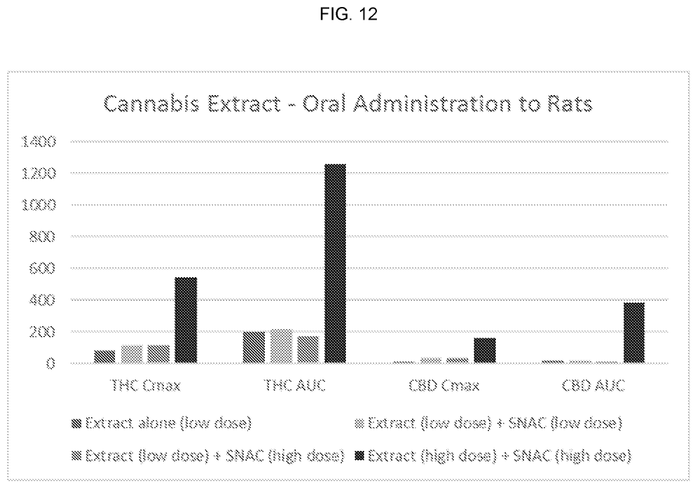

Over the dose range tested, Cₘₐₓ and AUC_{0-Tlast}, for THC was higher than for CBD. When administering the same cannabis extract (THC/CBD) dose (25 mg/kg total cannabinoid dose; 12.5 mg/kg THC/12.5 mg/kg CBD) with and without SNAC, for THC, a 1.4-fold Cₘₐₓ increase over cannabis alone was observed at SNAC doses of either 250 or 500 mg/kg. AUC was 1.1-fold greater in the 250 mg/kg SNAC group, but lower in the 500 mg/kg SNAC group, compared to the cannabis alone group. For CBD, 2.9-fold and 2.8-fold Cₘₐₓ increases over cannabis alone were observed at SNAC doses of either 250 or 500 mg/kg. AUC was lower in both groups, compared to the cannabis alone group. Increasing both the cannabis and SNAC doses 2-fold to 500 mg/kg SNAC and 50 mg/kg cannabis extract (25 mg/kg THC/25 mg/kg CBD), resulted in a 14.2-fold increase in the CBD Cₘₐₓ and a 6.9-fold increase in the THC Cₘₐₓ. AUC_{0-Tlast} for CBD and THC, were increased 22.1-fold and 6.3-fold, respectively (FIG. 11 and FIG. 12).

Over the dose range tested, Cₘₐₓ and AUC_{0-Tlast}, for THC was higher than for CBD. When administering the same cannabis extract (THC/CBD) dose in the presence of SNAC (250 mg/kg or 500 mg/kg), both THC and CBD Cₘₐₓ were increased 1.4-fold and 2.8-fold, respectively, over the cannabis alone group. AUC_{0-Tlast} were comparable. This observation suggests that a cannabis to SNAC ratio of 10:1 facilitates an increase in Cₘₐₓ, but increasing the ratio to 20:1 provides no additional benefit. Increasing both the cannabis and SNAC doses by 2-fold resulted in THC and CBD Cₘₐₓ increases of 6.9-fold and 14.2-fold, respectively, over the cannabis alone group. AUC_{0-Tlast} for THC and CBD increased by 6.3-fold and 22.1-fold, respectively, over the cannabis alone group. This is a greater than expected increase based on the near linear dose response observed for oral cannabis (Information for Health Care Providers—Cannabis and the Cannabinoids; Health Canada February 2013). Overall, these data suggest that SNAC enhances cannabis absorption when administered to rats by oral gavage.

### Example 6

Onset and duration of action of orally administered cannabis/NAC composition. This study was designed to assess the utility of the acid form of SNAC, N-[8-(2-hydroxybenzoyl) amino] caprylic acid (NAC), in enabling a rapid-acting oral form of cannabis.

Study Participant. One study participant was recruited to ingest cannabis compositions and record the onset, duration, and intensity of cannabis-induced euphoria and/or dysphoria. The study participant took part in two separate tests: 1) use of a control substance, which included cannabis concentrate oil in an herbal extract blend dissolved in aqueous ethanol, and 2) use of a test substance, which included the cannabis concentrate oil in an herbal extract blend dissolved in aqueous ethanol, as well as NAC.

Formulations. The selected cannabis concentrate oil is commercially available in a capsule and the contents of the capsule were provided to the participant in an ethanol solution. One capsule contains 9 mg CBD, 7.7 mg THC, herbal extract blend (Magnolia bark, Ashwagandha, Astragalus), and stearic acid (from vegetable oil), and the stated potency per capsule is: CBD 9.0 mg, THCA 0.0 mg and THC 7.6 mg. The formulation was selected because it provides a noticeable effect on user-reported “euphoria”, and the CBD content should ameliorate dysphoric effects if Test 2 delivers a very high dose of cannabinoids.

Methods. For the Control experiment, the participant mixed the cannabis concentrate with 15 ml (one tablespoon) of aqueous ethanol, and immediately swallowed the mixture.

For the Test experiment, the participant mixed the cannabis concentrate with 5 ml pre-mixed solution of aqueous ethanol and 100 mg NAC, and immediately swallowed the dissolved mixture.

For both the Control experiment and the Test experiment, the participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-5. Table 5 shows descriptions of euphoria and dysphoria levels for each scale value.

TABLE 5
Scale Values for Reporting
Euphoria and Dysphoria

| Scale | |
|---|---|
| Value | Description |
| 0 | No observed effect |
| 1 | Mild observed effect; |
| | possibly psychological |
| 2 | Definite but mild effect |
| 3 | Definite substantial effect |
| 4 | Strong effect |
| 5 | Intense effect |

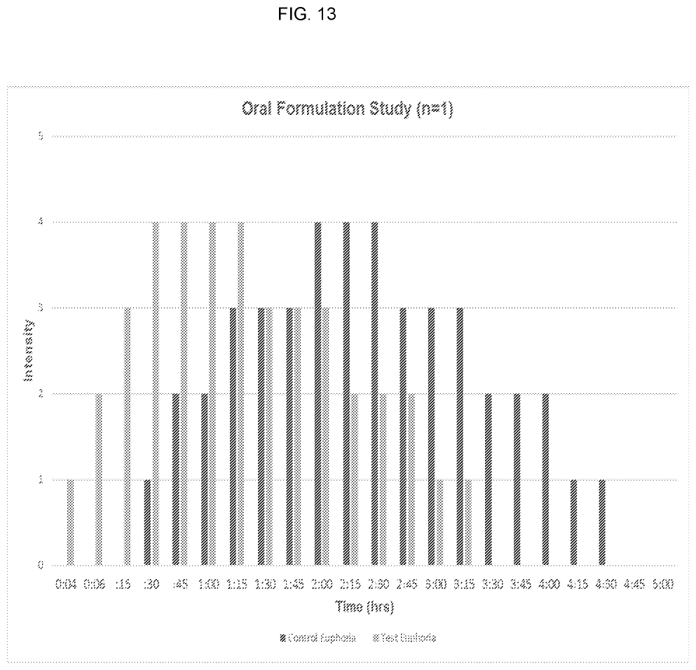

Results. The results shown below are scale values obtained for the participant in the control experiment (Table 6) and in the test experiment (Table 7). The values are plotted in FIG. 13.

TABLE 6
Control Experiment (n = 1)

| Actual | Time from | Observed | Observed |
|---|---|---|---|
| Time | Start | “Euphoria” | “Dysphoria” |
| 11:13 AM | 0:00 | (0-5) | (0-5) |
| 11:28 AM | :15 | 0 | 0 |
| 11:43 AM | :30 | 1 | 0 |
| 11:58 AM | :45 | 2 | 0 |
| 12:13 PM | 1:00 | 2 | 0 |
| 12:28 PM | 1:15 | 3 | 1 |
| 12:43 PM | 1:30 | 3 | 1 |
| 12:58 PM | 1:45 | 3 | 1 |
| 1:13 PM | 2:00 | 4 | 1 |
| 1:28 PM | 2:15 | 4 | 1 |
| 1:43 PM | 2:30 | 4 | 1 |
| 1:58 PM | 2:45 | 3 | 0 |
| 2:13 PM | 3:00 | 3 | 0 |
| 2:28 PM | 3:15 | 3 | 0 |
| 2:43 PM | 3:30 | 2 | 0 |
| 2:58 PM | 3:45 | 2 | 0 |
| 3:13 PM | 4:00 | 2 | 0 |
| 3:28 PM | 4:15 | 1 | 0 |
| 3:43 PM | 4:30 | 1 | 0 |
| 3:58 PM | 4:45 | 0 | 0 |
| 4:13 PM | 5:00 | 0 | 0 |

TABLE 7
Test Experiment (n = 1)

| Actual | Time from | Observed | Observed |
|---|---|---|---|
| Time | Start | “Euphoria” | “Dysphoria” |
| 11:20 AM | 0:00 | (0-5) | (0-5) |
| 11:24 AM | :04 | 1 | 0 |
| 11:26 AM | :06 | 2 | 0 |
| 11:35 AM | :15 | 3 | 1 |
| 11:50 AM | :30 | 4 | 0 |
| 12:05 PM | :45 | 4 | 0 |
| 12:20 PM | 1:00 | 4 | 0 |
| 12:35 PM | 1:15 | 4 | 0 |
| 12:50 PM | 1:30 | 3 | 0 |
| 1:05 PM | 1:45 | 3 | 0 |
| 1:20 PM | 2:00 | 3 | 0 |
| 1:35 PM | 2:15 | 2 | 0 |
| 1:50 PM | 2:30 | 2 | 0 |
| 2:05 PM | 2:45 | 2 | 0 |
| 2:20 PM | 3:00 | 1 | 0 |
| 2:35 PM | 3:15 | 1 | 0 |
| 2:50 PM | 3:30* | 0 | 0 |

*experiment ended

Onset: The participant reported euphoria within six minutes of ingesting the cannabis/NAC formulation (Test, Table 7 and FIG. 13). In contrast, the first time-point of euphoria reported by the participant after ingestion of the cannabis-only formulation (Control, Table 6 and FIG. 13) was forty-five minutes post-ingestion. By thirty minutes post-ingestion, the participant reported strong euphoria (scale value of 4) for the cannabis/NAC formulation. In contrast, thirty minutes after ingestion of the cannabis-only formulation, the participant only observed a mild effect that was possibly psychological (scale value of 1).

Intensity: The peak euphoria scale value after ingestion of both the cannabis-only (Control) and cannabis/NAC formulations (Test) was 4. However, peak intensity of euphoria was reached thirty minutes post-ingestion with the cannabis/NAC formulation (Test), whereas peak intensity of euphoria was reached two hours post-ingestion with the cannabis-only formulation (Control). Therefore, ingestion of the cannabis/NAC formulation led to a peak intensity of euphoria that occurred one hour and thirty minutes faster than when the cannabis-only formulation was ingested. The intensity of observed dysphoria was minimal for both the Test and Control, although the participant observed more mild dysphoria effects with the cannabis-only formulation (Control).

In summary, NAC, the add form of SNAC, behaves similarly to SNAC when included in an oral dosage formulation of cannabis. A cannabis/NAC formulation provides faster onset of action as compared to a cannabis-only formulation.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. In particular embodiments, a material effect would cause a statistically-significant reduction in an administration benefit when assessed in an experimental protocol disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. An oral formulation comprising (i) a rapid onset component comprising (a) vegetable matter and/or synthetic cannabinoids and (b) an N-acylated fatty amino acid or a salt thereof; and (ii) an extended action component comprising (a) vegetable matter and/or synthetic cannabinoids and (b) a sustained release system.

2. An oral formulation of claim 1 wherein the rapid onset component comprises a liquid.

3. An oral formulation of claim 1 wherein the liquid comprises water, ethanol, polyethylene glycols and polyvinyl alcohols.

4. An oral formulation of claim 1 wherein the extended action component comprises particles within the liquid.

5. An oral formulation of claim 4 wherein the particles comprise a release-controlling matrix, an enteric coating and/or a barrier layer.

6. An oral formulation of claim 5 wherein the particles comprise a release-controlling matrix selected from acrylic polymers, alkyl celluloses, vegetable oils and waxes.

7. An oral formulation of claim 5 comprising a release-controlling matrix and an enteric coating.

8. An oral formulation of claim 7 wherein the enteric coating is selected from acrylic polymers, celluloses, phthalate polymers and resins.

9. An oral formulation of claim 5 comprising a release-controlling matrix and a barrier layer.

10. An oral formulation of claim 9 wherein the barrier layer is selected from gellable polymers, natural gels, swellable polymers and erodible/slow dissolving polymers and gels.

11. An oral formulation of claim 1 wherein the extended action component comprises a liquid surrounded by the rapid onset component.

12. An oral formulation of claim 11 wherein the liquid is a vegetable oil.

13. An oral formulation of claim 11 wherein the liquid is within a gelcap.

14. An oral formulation of claim 13 wherein the gelcap is coated with a rapid onset shell.

15. An oral formulation of claim 1 in the form of a tablet.

16. An oral formulation of claim 15 wherein the rapid onset component of the tablet surrounds the extended action component of the tablet.

17. An oral formulation of claim 15 wherein the rapid onset component comprises N-acylated fatty amino acid or a salt thereof, EDTA, citric acid, bile salts, chitosan, SNAC, NAC, SDS, medium chain fatty acids and acyklcarnitines.

18. An oral formulation of claim 15 wherein the extended action component comprises a release-controlling matrix, an enteric coating and/or a barrier layer.

19. An oral formulation of claim 18 wherein the extended action component comprises a release-controlling matrix selected from acrylic polymers, alkyl celluloses, vegetable oils and waxes.

20. An oral formulation of claim 18 comprising a release-controlling matrix and an enteric coating.

21. An oral formulation of claim 20 wherein the enteric coating is selected from acrylic polymers, celluloses, phthalate polymers and resins.

22. An oral formulation of claim 20 comprising a release-controlling matrix and a barrier layer.

23. An oral formulation of claim 22 wherein the barrier layer is selected from gellable polymers, natural gels, swellable polymers and erodible/slow dissolving polymers and gels.

24. An oral formulation of claim 1 comprising an acrylic polymer selected from one or more of acrylic acid and methacrylic acid copolymer, aminoalkyl methacrylate copolymer, cyanoethyl methacrylate, ethoxyethyl methacrylate, glycidyl methacrylate copolymer, methacrylic acid alkylamide copolymer, methyl methacrylate copolymer, poly(acrylic acid), poly(methacrylic acid), poly(methacrylic acid anhydride), poly(methyl methacrylate), poly(methyl methacrylate) copolymer, polyacrylamide, and polymethacrylate, methyl methacrylate.

25. An oral formulation of claim 1 comprising an alkyl cellulose selected from methyl cellulose and/or ethyl cellulose.

26. An oral formulation of claim 1 comprising vegetable oil selected from one or more of sesame oil, palm oil, palm hydrogenated oil, corn germ hydrogenated oil, castor hydrogenated oil, cotton-seed oil, olive oil, peanut oil, palm olein oil, and palm stearin oil.

27. An oral formulation of claim 1 comprising a wax selected from one or more of beeswax, glycowax, castor wax, and carnauba wax.

28. An oral formulation of claim 1 comprising a cellulose selected from one or more of hydroxyalkyl cellulose, methyl cellulose, ethyl cellulose, hydroxyethyl cellulose, hydroxypropyl cellulose, hydroxypropylmethyl cellulose (HPMC), hydroxypropylethyl cellulose, hydroxypropylpropyl cellulose, and hydroxypropylbutyl cellulose.

29. An oral formulation of claim 1 comprising a phthalate polymer selected from one or more of cellulose acetate hexahydrophthalate, cellulose acetate phthalate (CAP), cellulose propionate phthalate, hydroxypropyl methylcellulose hexahydrophthalate, hydroxypropyl methylcellulose phthalate (HPMCP), and polyvinyl acetate phthalate (PVAP).

30. An oral formulation of claim 1 comprising a resin selected from one or more of zein, gelatin, shellac and acacia.

31. An oral formulation of claim 1 comprising a gellable polymer selected from one or more of guar gum, mannose sugar, galactose sugar, hydropropyl guar, carboxymethyl guar, carboxymethylhydroxypropyl guar, hydroxyethylcellulose, hydroxypropylcellulose, carboxymethyl hydroxyethylcellulose, xanthan, diutan, scleroglucan, polyacrylamide, polyvinyl alcohol, polyethylene glycol, polypropylene glycol, and a polyacrylate polymer.

32. An oral formulation of claim 1 comprising a swellable polymer comprising one or more of poly(acrylic acid), poly(alkylene oxide), poly(vinyl alcohol), poly(vinyl pyrrolidone), polyurethane hydrogel, maleic anhydride polymer, cellulose, hydroxymethylcellulose, hydroxyethyl cellulose, hydroxypropyl cellulose, hydroxypropylmethyl cellulose, carboxymethyl cellulose, dextran, xanthan gum, gellan gum, welan gum, rhamsan gum, sodium alginate, calcium alginate, chitosan, gelatin, maltodextrin, starch, hydrolyzed starch polyacrylonitrile graft copolymers, and/or starch-acrylate-acrylamide copolymers.

33. An oral formulation of claim 1 comprising an erodible polymer comprising one or more of polyethylene oxide, polyethylene oxide water soluble resins, glyceryl fatty acid esters, hydrogenated castor oil, hydroxyethyl cellulose, hydroxypropyl cellulose, hydroxypropyl methylcellulose, ethylhydroxy ethylcellulose, methylethyl cellulose, carboxymethyl cellulose, carboxymethyl ethylcellulose, pullulan, polyvinyl pyrrolidone, polyvinyl alcohol, and polyvinyl acetate.

34. An oral formulation of claim 1 comprising a botanical product.

35. An oral formulation of claim 1 wherein the vegetable matter is derived from Calophyllum brasiliense, Calophyllum caledonicurn, Calophyllum inophyllum, Calophyllum soulattri, Uncaria tomentosa, Thymus vulgaris, Matricaria recutita, Salix alba, Calendula officinalis, Usnea barbata, Ligusticum porterii-osha, Gaultheria procumbens, Camellia sinensis, Vaccinium myrtillus, Melissa officinalis, Allium sativum, Camellia sinensis, Krameria triandra, Punica granatum, Viburnum plicatum, Nicotiana tabacum, Duboisia hopwoodii, Asclepias syriaca, Curcumalonga, Hypericum perforatum, Polygonum cuspidatum, Vitis spp., Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., Mentha spp., Cannabis sativa, Cannabis indica, Cannabis ruderalis and/or Acer spp, or an extract thereof.

36. An oral formulation of claim 1 wherein the vegetable matter is derived from cannabis.

37. An oral formulation of claim 1 wherein the vegetable matter is derived from Cannabis sativa, Cannabis ruderalis, or Cannabis indica.

38. An oral formulation of claim 1 comprising a cannabis extract.

39. An oral formulation of claim 1 comprising cannabinoids.

40. An oral formulation of claim 1 comprising Δ9-Tetrahydrocannabinol (THC) and cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), and/or mixtures thereof.

41. An oral formulation of claim 1 comprising curcumin, hypericin, resveratrol, capsaicin, reserpine, vinblastine, hesperidin, naringin, rutin, quercitrin, catechin eugenol, limonene, linalool or nicotine.

42. The oral formulation of claim 1 wherein the one or more synthetic cannabinoids comprise THC, CBD, CBG, CBC, CBN, CBDL, CBL, CBV, THCV, CBDV), CBCV, CBGV, CBGM, cannabinerolic acid, CBDA, CBNV, CBO, THCA, THCVA, or mixtures thereof.

43. The oral formulation of claim 1 wherein the one or more synthetic cannabinoids comprise a derivative and/or analog of a synthetic cannabinoid of claim 42 or a mixture thereof.

44. The oral formulation of claim 1 wherein the one or more synthetic cannabinoids comprise 3-carbamoyl-2-pyridone or its derivatives and/or analogs; pyrimidine derivatives and/or analogs; carenadiol or its derivatives and/or analogs; cannabinoid carboxylic acids or their derivatives and/or analogs; pyrido[3,2-E][1,2,4]triazolo[4,3-C]pyrimidine or its derivatives and/or analogs; tetrahydro-pyrazolo[3,4-C] pyridine or its derivatives and/or analogs; bicyclo[3.1.1]heptan-2-one cannabinoid or its derivatives and/or analogs; resorcinol or its derivatives and/or analogs; dexanbinol compounds or their derivatives and/or analogs; cannabimimetic lipid amide compounds or their derivatives and/or analogs; nabilone or its derivatives and/or analogs; 2-oxoquinolone compounds or their derivatives and/or analogs disclosed; or 3,4-diaryl-4,5-dihydro-(h)-pyrazole-1-carboxamide or its derivatives and/or analogs.

45. An oral formulation of claim 1 comprising flavonoid compounds, terpenes, or terpenoids.

46. An oral formulation of claim 1 wherein the N-acylated fatty amino acid comprises one or more of Compounds 1-XXXV (FIG. 5), or Compounds a-r (FIG. 6).

47. An oral formulation of claim 1 wherein the N-acylated fatty amino acid comprises monosodium-N-salicyloyl-8-aminocaprylate, disodium-N-salicyloyl-8-aminocaprylate, and N-(salicyloyl)-8-aminocaprylic acid.

48. An oral formulation of claim 1 wherein the N-acylated fatty amino acid or a salt thereof comprises 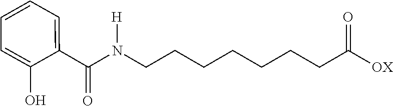
wherein X and Z are independently H, a monovalent cation, a divalent metal cation, or an organic cation.

49. An oral formulation of claim 48 wherein the monovalent cation comprises sodium or potassium.

50. An oral formulation of claim 48 wherein the metal cation comprises calcium or magnesium.

51. An oral formulation of claim 48 wherein the organic cation comprises ammonium or tetramethylammonium.

52. An oral formulation of claim 48 wherein X is H.

53. An oral formulation of claim 48 wherein X is a monovalent cation comprising sodium or potassium.

54. An oral formulation of claim 48 wherein X is a divalent metal cation comprising calcium or magnesium.

55. An oral formulation of claim 48 wherein X is an organic cation comprising ammonium or tetramethylammonium.

56. An oral formulation of claim 48 wherein Z is H.

57. An oral formulation of claim 48 wherein Z is a monovalent cation comprising sodium or potassium.

58. An oral formulation of claim 48 wherein Z is a divalent cation comprising calcium or magnesium.

59. An oral formulation of claim 48 wherein X is H and Z is H.

60. An oral formulation of claim 48 wherein X is H and Z is sodium.

61. An oral formulation of claim 48 wherein X is sodium and Z is sodium.

62. An oral formulation of claim 1 wherein the N-acylated fatty amino acid provides an administration benefit.

63. An oral formulation of claim 62 wherein the administration benefit is a dose-dependent administration benefit.

64. An oral formulation of claim 63 wherein the dose-dependent administration benefit is at a dose of 100-200 mg.

65. An oral formulation of claim 62 wherein the administration benefit comprises one or more of increased absorption of a measured component of vegetable matter, increased bioavailability of a measured component of vegetable matter, faster onset of action of a measured component of vegetable matter, higher peak concentrations of a measured component of vegetable matter, faster time to peak concentrations of a measured component of vegetable matter, increased subjective therapeutic efficacy, increased objective therapeutic efficacy, improved taste, and improved mouthfeel as compared to a control oral formulation without the N-acylated fatty amino acid.

66. An oral formulation of claim 1 wherein the oral formulation is a medicinal oral formulation.

67. An oral formulation of claim 1 wherein the oral formulation is a nutritional supplement.

68. An oral formulation of claim 1 comprising a surfactant, detergent, azone, pyrrolidone, glycol or bile salt.

69. An oral formulation of claim 1 comprising a therapeutically effective amount of vegetable matter.

70. An oral formulation of claim 69 wherein the therapeutically effective amount treats a symptom of acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Grave's disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, and withdrawal.

71. An oral formulation of claim 1 comprising vitamins or minerals.

72. An oral formulation of claim 1 comprising vitamins and minerals.

73. An oral formulation of claim 71 wherein the vitamins are selected from one or more of Vitamin A, Vitamin B1, Vitamin B6, Vitamin B12, Vitamin C, Vitamin D, Vitamin E, or Vitamin K.

74. An oral formulation of claim 71 wherein the minerals are selected from one or more of calcium, chromium, iodine, iron, magnesium, selenium or zinc.

75. An oral formulation of claim 1 wherein the oral formulation is swallowable or chewable.

76. An oral formulation of any of claim 1 wherein the oral formulation is liquid or solid.

77. An oral formulation of claim 1 wherein the oral formulation is a solution, suspension, or spray.

78. An oral formulation of claim 1 wherein the oral formulation is a tablet, capsule or sachet.

79. An oral formulation of claim 1 wherein the oral formulation is flavored.

80. A method of preparing an oral formulation of cannabis having a faster onset of action, wherein the method comprises adding an absorption enhancer to the oral formulation of cannabis and wherein the oral formulation of cannabis has a faster onset of action than an oral formulation of cannabis without an absorption enhancer.

81. The method of claim 80, wherein the absorption enhancer is an N-acylated fatty amino acid or a salt thereof.

82. The method of claim 81, wherein the N-acylated fatty amino acid or a salt thereof comprises 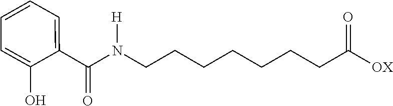
wherein X and Z are independently H, a monovalent cation, a divalent metal cation, or an organic cation.

83. The method of claim 81 wherein the N-acylated fatty amino acid is selected from monosodium-N-salicyloyl-8-aminocaprylate, disodium-N-salicyloyl-8-aminocaprylate, and N-(salicyloyl)-8-aminocaprylic acid.

84. A method of treating a subject in need thereof comprising administering a therapeutically effective amount of an oral formulation of claim 1 to the subject thereby treating the subject in need thereof.

85. A method of claim 84 wherein the therapeutically effective amount provides an effective amount, a prophylactic treatment, and/or a therapeutic treatment.

86. A method of reducing or eliminating one or more symptoms of a disease or disorder in a human subject,
wherein said method comprises delivering a therapeutically effective amount of an oral formulation of claim 1 to the subject, thereby reducing or eliminating one or more symptoms of the disease or disorder, and
wherein said disease or disorder is acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Grave's disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.
